SEQUENCE LISTING (71 sequences)

SEQ ID NO: 1 - protein, 976 aa, Chlamydia muridarum
MKKLFFFVLIGSSILGFTREVPPSILLKPILNPYHMTGLFFPKVNLLGDTHNLTDYHLDNLKCILACLQRTPYEGAAFTVTDYLGFSDTQKDGIFCFKNLTPESGGVIGSPTQNTPTIKIHNTIGPVLFENNTCHRLWTQTDPENEGNKAREGGAIHAGDVYISNNQNLVGFIKNFAYVQGGAISANTFAYKENKSSFLCLNNSCIQTKTGGKGGAIYVSTSCSFENNNKDLLFIQNSGCAGGAIFSPTCSLIGNQGDIVFYSNHGFKNVDNATNESGDGGAIKVTTRLDITNNGSQIFFSDNISRNFGGAIHAPCLHLVGNGPTYFTNNIANHTGGAIYITGTETSKISADHHAIIFDNNISANATNADGSSSNTNPPHRNAITMDNSAGGIELGAGKSQNLIFYDPIQVTNAGVTVDFNKDASQTGCVVFSGATVLSADISQANLQTKTPATLTLSHGLLCIEDRAQLTVNNFTQTGGIVALGNGAVLSSYQHSTTDATQTPPTTTTTDASVTLNHIGLNLPSILKDGAEMPLLWVEPISTTQGNTTTYTSDTAASFSLNGATLSLIDEDGNSPYENTDLSRALYAQPMLAISEASDNQLQSESMDFSKVNVPHYGWQGLWTWGWAKTENPTTTPPATITDPKKANQFHRTLLLTWLPAGYIPSPKHKSPLIANTLWGNILFATENLKNSSGQELLDRPFWGITGGGLGMMVYQEPRKDHPGFHMHTSGYSAGMITGNTHTFSLRFSQSYTKLNERYAKNYVSSKNYSCQGEMLLSLQEGLMLTKLIGLYSYGNHNSHHFYTQGEDLSSQGEFHSQTFGGAVFFDLPLKPFGRTHILTAPFLGAIGMYSKLSSFTEVGAYPRTFITETPLINVLIPIGVKGSFMNATHRPQAWTVELAYQPVLYRQEPSISTQLLAGKGMWFGHGSPASRHALAYKISQKTQLLRFATLQLQYHGYYSSSTFCNYLNGEVSLRF

SEQ ID NO: 2 - protein, 964 aa, Chlamydia trachomatis
MKKAFFFFLIGNSLSGLAREVPSRIFLMPNSVPDPTKESLSNKISLTGDTHNLTNCYLDNLRYILAILQKTPNEGAAVTITDYLSFFDTQKEGIYFAKNLTPESGGAIGYASPNSPTVEIRDTIGPVIFENNTCCRLFTWRNPYAADKIREGGAIHAQNLYINHNHDVVGFMKNFSYVQGGAISTANTFVVSENQSCFLFMDNICIQTNTAGKGGAIYAGTSNSFESNNCDLFFINNACCAGGAIFSPICSLTGNRGNIVFYNNRCFKNVETASSEASDGGAIKVTTRLDVTGNRGRIFFSDNITKNYGGAIYAPVVTLVDNGPTYFINNIANNKGGAIYIDGTSNSKISADRHAIIFNENIVTNVTNANGTSTSANPPRRNAITVASSSGEILLGAGSSQNLIFYDPIEVSNAGVSVSFNKEADQTGSVVFSGATVNSADFHQRNLQTKTPAPLTLSNGFLCIEDHAQLTVNRFTQTGGVVSLGNGAVLSCYKNGTGDSASNASITLKHIGLNLSSILKSGAEIPLLWVEPTNNSNNYTADTAATFSLSDVKLSLIDDYGNSPYESTDLTHALSSQPMLSISEASDNQLQSENIDFSGLNVPHYGWQGLWTWGWAKTQDPEPASSATITDPQKANRFHRTLLLTWLPAGYVPSPKHRSPLIANTLWGNMLLATESLKNSAELTPSGHPFWGITGGGLGMMVYQDPRENHPGFHMRSSGYSAGMIAGQTHTFSLKFSQTYTKLNERYAKNNVSSKNYSCQGEMLFSLQEGFLLTKLVGLYSYGDHNCHHFYTQGENLTSQGTFRSQTMGGAVFFDLPMKPFGSTHILTAPFLGALGIYSSLSHFTEVGAYPRSFSTKTPLINVLVPIGVKGSFMNATHRPQAWTVELAYQPVLYRQEPGIAAQLLASKGIWFGSGSPSSRHAMSYKISQQTQPLSWLTLHFQYHGFYSSSTFCNYLNGEIALRF

SEQ ID NO: 3 - DNA, 2931 bp, Chlamydia muridarum
atgaaaaaactgttcttttttgtccttattggaagctctatactgggatttactcgagaagtccctccttcgattcttttaaagcctatactaaatccataccatatgaccgggttattttttcccaaggttaatttgcttggagacacacataatctcactgattaccatttggataatctaaaatgcattctggcttgcctacaaagaactccttatgaaggagctgctttcacagtaaccgattacttaggtttttcagatacacaaaaggatggtattttttgttttaaaaatcttactccagagagtggaggggttattggttccccaactcaaaacactcctactataaaaattcataatacaatcggccccgttcttttcgaaaataatacctgtcatagactgtggacacagaccgatcccgaaaatgaaggaaacaaagcacgcgaaggcggggcaattcatgctggggacgtttacataagcaataaccagaaccttgtcggattcataaagaactttgcttatgttcaaggtggagctattagtgctaatacttttgcctataaagaaaataaatcgagctttctttgcctaaataactcttgtatacaaactaagacgggagggaaaggtggtgctatttacgttagtacgagctgctctttcgagaacaataacaaggatctgcttttcatccaaaactccggctgtgcaggaggagctatcttctctccaacctgttctctaataggaaaccaaggagatattgttttttacagcaaccacggttttaaaaatgttgataatgcaactaacgaatctggggatggaggagctattaaagtaactacccgcttggacatcaccaataatggtagtcaaatctttttttctgataatatctcaagaaattttggaggagctattcatgctccttgtcttcatcttgttggtaatgggccaacctattttacaaacaatatagctaatcacacaggtggggctatttatataacaggaacagaaacctcaaagatttctgcagatcaccatgctattatttttgataataacatttctgcaaacgccaccaatgcggacggatctagcagcaacactaatcctcctcacagaaatgcgatcactatggacaattccgctggaggaatagaacttggtgcagggaagagccagaatcttattttctatgatcctattcaagtgacgaatgctggagttaccgtagacttcaataaggatgcctcccaaaccggatgtgtagttttctctggagcgactgtcctttctgcagatatttctcaggctaatttgcaaactaaaacacctgcaacgcttactctcagtcacggtcttctgtgtatcgaagatcgtgctcagctcacagtgaacaattttacacaaacaggagggattgtagccttaggaaatggagcagttttaagcagctaccaacacagcactacagacgccactcaaactccccctacaaccaccactacagatgcttccgtaactcttaatcacattggattaaatctcccctctattcttaaggatggagcagagatgcctctattatgggtagaacctataagcacaactcaaggtaacactacaacatatacgtcagataccgcggcttccttctcattaaatggagccacactctctctcattgatgaagatggaaattctccctatgaaaacacggacctctctcgtgcattgtacgctcaacctatgctagcaatttctgaggccagtgataaccaattgcaatccgaaagcatggacttttctaaagttaatgttcctcactatggatggcaaggactttggacctgggggtgggcaaaaactgaaaatccaacaacaactcctccagcaacaattactgatccgaaaaaagctaatcagtttcatagaactttattattaacgtggctccctgctggttatatccccagccctaaacataaaagccctttaatagctaataccttgtgggggaatatactttttgcaacggaaaacttaaaaaatagctcagggcaagaacttcttgatcgtcctttctggggaattacaggagggggcttggggatgatggtctatcaagaacctagaaaagaccatcctggattccacatgcatacctccggatattcagcaggaatgattacaggaaacacacataccttctcattacgattcagccagtcctatacaaaactcaatgaacgttatgccaagaactatgtgtcttctaaaaattactcttgccaaggggaaatgcttttgtccttacaagaaggactcatgctgactaaactaattggtctctatagttatgggaatcacaacagccaccatttctatacccaaggagaagacctatcgtctcaaggggagttccatagtcagacttttggaggggctgtcttttttgatctacctctgaaaccttttggaagaacacacatacttacagctcctttcttaggtgccattggtatgtattctaagctgtctagctttacagaagtaggagcctatccaagaacctttattacagaaacgcctttaatcaatgtcctgattcctatcggagtaaaaggtagcttcatgaatgccacccatagacctcaggcctggactgtagagcttgcttaccaacctgttctttacagacaagaacctagtatctctacccaattactcgctggtaaaggtatgtggtttgggcatggaagtcctgcatctcgccacgctctagcttataaaatttcacagaaaacacagcttttgcgatttgcaacacttcaactccagtatcacggatactattcgtcttccactttctgtaattatctgaatggagaggtatctttacgtttctaa

SEQ ID NO: 4 - DNA, 2895 bp, Chlamydia trachomatis
atgaaaaaagcgtttttctttttccttatcggaaactccctatcaggactagctagagaggttccttctagaatctttcttatgcccaactcagttccagatcctacgaaagagtcgctatcaaataaaattagtttgacaggagacactcacaatctcactaactgctatctcgataacctacgctacatactggctattctacaaaaaactcccaatgaaggagctgctgtcacaataacagattacctaagcttttttgatacacaaaaagaaggtatttattttgcaaaaaatctcacccctgaaagtggtggtgcgattggttatgcgagtcccaattctcctaccgtggagattcgtgatacaataggtcctgtaatctttgaaaataatacttgttgcagactatttacatggagaaatccttatgctgctgataaaataagagaaggcggagccattcatgctcaaaatctttacataaatcataatcatgatgtggtcggatttatgaagaacttttcttatgtccaaggaggagccattagtaccgctaatacctttgttgtgagcgagaatcagtcttgttttctctttatggacaacatctgtattcaaactaatacagcaggaaaaggtggcgctatctatgctggaacgagcaattcttttgagagtaataactgcgatctcttcttcatcaataacgcctgttgtgcaggaggagcgatcttctcccctatctgttctctaacaggaaatcgtggtaacatcgttttctataacaatcgctgctttaaaaatgtagaaacagcttcttcagaagcttctgatggaggagcaattaaagtaactactcgcctagatgttacaggcaatcgtggtaggatcttttttagtgacaatatcacaaaaaattatggcggagctatttacgctcctgtagttaccctagtggataatggccctacctactttataaacaatatcgccaataataaggggggcgctatctatatagacggaaccagtaactccaaaatttctgccgaccgccatgctattatttttaatgaaaatattgtgactaatgtaactaatgcaaatggtaccagtacgtcagctaatcctcctagaagaaatgcaataacagtagcaagctcctctggtgaaattctattaggagcagggagtagccaaaatttaattttttatgatcctattgaagttagcaatgcaggggtctctgtgtccttcaataaggaagctgatcaaacaggctctgtagtattttcaggagctactgttaattctgcagattttcatcaacgcaatttacaaacaaaaacacctgcaccccttactctcagtaatggttttctatgtatcgaagatcatgctcagcttacagtgaatcgattcacacaaactgggggtgttgtttctcttgggaatggagcagttctgagttgctataaaaatggtacaggagattctgctagcaatgcctctataacactgaagcatattggattgaatctttcttccattctgaaaagtggtgctgagattcctttattgtgggtagagcctacaaataacagcaataactatacagcagatactgcagctaccttttcattaagtgatgtaaaactctcactcattgatgactacgggaactctccttatgaatccacagatctgacccatgctctgtcatcacagcctatgctatctatttctgaagctagcgataaccagctacaatcagaaaatatagatttttcgggactaaatgtccctcattatggatggcaaggactttggacttggggctgggcaaaaactcaagatccagaaccagcatcttcagcaacaatcactgatccacaaaaagccaatagatttcatagaaccttactactaacatggcttcctgccgggtatgttcctagcccaaaacacagaagtcccctcatagctaacaccttatgggggaatatgctgcttgcaacagaaagcttaaaaaatagtgcagagctgacacctagtggtcatcctttctggggaattacaggaggaggactaggcatgatggtttaccaagatcctcgagaaaatcatcctggattccatatgcgctcttccggatactctgcggggatgatagcagggcagacacacaccttctcattgaaattcagtcagacctacaccaaactcaatgagcgttacgcaaaaaacaacgtatcttctaaaaattactcatgccaaggagaaatgctcttctcattgcaagaaggtttcttgctgactaaattagttgggctttacagctatggagaccataactgtcaccatttctatactcaaggagaaaatctaacatctcaagggacgttccgcagtcaaacgatgggaggtgctgtcttttttgatctccctatgaaaccctttggatcaacgcatatactgacagctccctttttaggtgctcttggtatttattctagcctgtctcactttactgaggtgggagcctatccgcgaagcttttctacaaagactcctttgatcaatgtcctagtccctattggagttaaaggtagctttatgaatgctacccacagacctcaagcctggactgtagaattggcataccaacccgttctgtatagacaagaaccagggatcgcagcccagctcctagccagtaagggtatttggttcggtagtggaagcccctcatcgcgtcatgccatgtcctataaaatctcacagcaaacacaacctttgagttggttaactctccatttccagtatcatggattctactcctcttcaaccttctgtaattatctcaatggggaaattgctctgcgattctag

SEQ ID NO: 5 - protein, 1025 aa, Chlamydia muridarum
MTRRILPLSLVFIPLSCISASETDTLKLPNLTFGGREIEFIVTPPSSIAAQYITYANVSNYRGNFTISSCTQDQWFSRGLSTTNSSGAFVESMTSFTAIDNADLFFCNNYCTHQGGGGAINATGLISFKNNQNILFYNNTTIGTQFTGVALRTERNRGGALYGSSIELINNHSLNFINNTSGDMGGAVSTIQNLVIKNTSGIVAFENNHTTDHIPNTFATILARGGAVGCQGACEISHNTGPVVFNSNYGGYGGAISTGGQCIFRDNKDKLIFINNSALGWHNTSAQGNGAVISAGGEFGLLNNKGPIYFENNNASYIAGAISCNNLNFQENGPIYFLNNSALYGGAFHLFASPAANYIHTGSGDIIFNNNTELSTTGMSAGLRKLFYIPGTTNNNPITLSLGAKKDTRIYFYDLFQWGGLKKANTPPENSPHTVTINPSDEFSGAVVFSYKNISSDLQAHMIASKTHNQIKDSPTTLKFGTMSIENGAEFEFFNGPLTQESTSLLALGQDSILTVGKDASLTITHLGIILPGLLNDQGTTAPRIRVNPQDMTQNTNSNQAPVSTENVATQKIFFSGLVSLVDENYESVYDSCDLSRGKANQPILHIETTNDAQLSNDWKNTLNTSLYSLPHYGYQGLWTSNWMTTTRTVSLTNSTETQTANNSIQEQKNTSETFDSNSTTTAKIPSIRASTGGTTPLATTDVTVTRHSLVVSWTPIGYIADPARRGDLIANNLVSSGRNTTLYLRSLLPDDSWFALQGSAATLFTKQQKRLDYHGYSSASKGYAISSQASGAHGHKFLFSFSQSSDTMKEKRTNNKISSRYYLSALCFEQPMFDRIALIGAAAYNYGTHKTYNFYGTKKFSKGNFHSTTLGGSLRCELRDSMPFQSIMLTPFIQALISRTEPASIQEQGDLARLFSLKQPHTAVVSPIGIKGVYSSNKWPTVSCEMEVAYQPTLYWKRPILNTVLIKNNGSWETTNTPLAKHSFYGRGSSSLKFSYLKLFANYQAQVATSTVSHYMNAGGALVF

SEQ ID NO: 6 - protein, 1034 aa, Chlamydia trachomatis
MIKRTSLSFACLSFFYLSTISILQANETDTLQFRRFTFSDREIQFVLDPASLITAQNIVLSNLQSNGTGACTISGNTQTQIFSNSVNTTADSGGAFDMVTTSFTASDNANLLFCNNYCTHNKGGGAIRSGGPIRFLNNQDVLFYNNISAGAKYVGTGDHNEKNRGGALYATTITLTGNRTLAFINNMSGDCGGAISADTQISITDTVKGILFENNHTLNHIPYTQAENMARGGAICSRRDLCSISNNSGPIVFNYNQGGKGGAISATRCVIDNNKERIIFSNNSSLGWSQSSSASNGGAIQTTQGFTLRNNKGSIYFDSNTATHAGGAINCGYIDIRDNGPVYFLNNSAAWGAAFNLSKPRSATNYIHTGTGDIVFNNNVVFTLDGNLLGKRKLFHINNNEITPYTLSLGAKKDTRIYFYDLFQWERVKENTSNNPPSPTSRNTITVNPETEFSGAVVFSYNQMSSDIRTLMGKEHNYIKEAPTTLKFGTLAIEDDAELEIFNIPFTQNPTSLLALGSGATLTVGKHGKLNITNLGVILPIILKEGKSPPCIRVNPQDMTQNTGTGQTPSSTSSISTPMIIFNGRLSIVDENYESVYDSMDLSRGKAEQLILSIETTNDGQLDSNWQSSLNTSLLSPPHYGYQGLWTPNWITTTYTITLNNNSSAPTSATSIAEQKKTSETFTPSNTTTASIPNIKASAGSGSGSASNSGEVTITKHTLVVNWAPVGYIVDPIRRGDLIANSLVHSGRNMTMGLRSLLPDNSWFALQGAATTLFTKQQKRLSYHGYSSASKGYTVSSQASGAHGHKFLLSFSQSSDKMKEKETNNRLSSRYYLSALCFEHPMFDRIALIGAAACNYGTHNMRSFYGTKKSSKGKFHSTTLGASLRCELRDSMPLRSIMLTPFAQALFSRTEPASIRESGDLARLFTLEQAHTAVVSPIGIKGAYSSDTWPTLSWEMELAYQPTLYWKRPLLNTLLIQNNGSWVTTNTPLAKHSFYGRGSHSLKFSHLKLFANYQAEVATSTVSHYINAGGALVF

SEQ ID NO: 7 - DNA, 3078 bp, Chlamydia muridarum
atgactcgcagaattctccctctttcacttgttttcattcctttatcttgtatttcggccagtgaaaccgatacactcaaacttccgaacttgacttttggtggtagagagattgaattcatagttactccgcctagctccattgctgctcaatacatcacttacgcaaatgtttctaattatagagggaactttactatttcaagttgtacgcaggatcaatggttttcgagaggtttaagcactacaaactctagtggagcttttgttgagtctatgacttctttcacagccattgacaatgcagacttgtttttttgtaacaattattgcacccatcagggaggagggggagctataaatgctacaggccttattagctttaaaaacaaccaaaacatattgttctataataatacaactattggaactcaatttacaggagtagcattaagaaccgaaaggaatcgcggaggggctttatacggatcaagcatcgagctaattaataatcatagcttaaattttatcaataacacttctggggatatgggaggagccgtatccacaatccaaaacctagttatcaaaaatacgtccggaatagttgcttttgaaaataaccatactactgatcacatacccaacacatttgctacaattcttgctcgaggaggagctgttggctgccaaggtgcctgcgaaatctcacacaatactggtccggtagtcttcaattccaactatggaggatacggaggagctatcagcaccgggggacagtgtatttttagagataataaggataagcttatttttataaataatagcgctttaggatggcataacactagtgctcaaggaaatggagcagttataagcgcaggaggagagtttggtcttctaaataataaaggccctatctactttgagaataataatgcctcatacatagcaggagctatttcctgcaacaaccttaattttcaagaaaatggtcctatctattttcttaataattcggctctgtatggaggagcttttcacctatttgcaagcccagctgcgaactatattcatactggctctggggatattatcttcaacaataatacagagctttcaactaccggaatgtcagcaggtttgcgaaaacttttttatattcctggaacaaccaacaataaccctatcaccctatctcttggtgctaagaaagatactcgcatctatttttatgatctttttcaatggggaggcttaaaaaaagctaatacaccccctgaaaatagcccgcacaccgttaccatcaatccttcggatgagttctctggcgctgttgtgttttcatacaaaaacatatccagtgatctacaagctcacatgattgccagtaaaactcataaccaaattaaagactcccccactaccttgaagtttgggactatgtccatagaaaatggcgcagagtttgaatttttcaatggccctcttactcaagaaagcactagccttcttgctttaggacaagattctattcttactgtagggaaagacgcttctctcactattacgcatcttggaatcattttgccaggtcttctcaatgaccaaggtactacagctccacgtattcgtgttaatccccaagatatgacacagaatacaaactctaaccaagctccagtaagcacagagaacgtggcaactcaaaagatctttttctccggtcttgtctcgttagtagatgaaaattacgaatcagtttatgacagctgcgacctatcccgaggaaaagcaaatcaacccattttacatatcgaaacgactaatgatgcgcagttaagcaatgattggaaaaacactctcaatacctcgctatattctttaccacattacggataccaaggactctggacatctaattggatgacaaccacccgtacggtctctcttaccaatagtacagagactcaaacagccaacaattctattcaagaacaaaaaaacactagcgaaacttttgattccaacagtacaactacagctaagattccttccattagagcttctacaggaggaacaactcctcttgctacaacggacgtaacagtcactagacactccttagtagtgagctggaccccaatcggatatatagcagatcctgctcgtagaggggatcttattgcgaataatttagtgtcttctggaagaaatacaaccctgtacttacgttcattactaccagatgactcttggttcgctttacaaggatctgcagctacgctattcaccaaacagcagaaacgcttagattatcacggatattcttctgcatcgaaaggatatgctatatcttcacaagcatcaggagcacacggacataagtttttattttccttttcccaatcctccgacacaatgaaagagaaacgtaccaataataaaatttcttctcgttattatctctccgctctgtgttttgaacaacctatgtttgatcgtatcgctcttattggagcagctgcttataactatggtactcataaaacatataacttctatggaacgaaaaagttttctaaagggaactttcactctacgactctggggggctctctacgttgcgaactgcgggatagtatgcctttccaatcgattatgttgacaccattcattcaagctctcatctcccgaacagagcctgcatctatccaggagcagggagacctggctagattattttcgttaaaacaaccacatacagctgttgtttctccaataggaattaaaggtgtttattcttcgaataaatggccaactgtatcctgcgaaatggaggtagcataccagcctactctttactggaagcgccccattcttaataccgttttaatcaaaaacaatggttcttgggaaacaacaaacactcctttagctaagcattccttttatgggagaggatcatcttctctaaaattctcttatcttaaactattcgctaattatcaagcgcaggtggctacttctacagtctcacactacatgaatgcaggaggggctctggtcttttaa

SEQ ID NO: 8 - DNA, 3105 bp, Chlamydia trachomatis
atgattaaaagaacttctctatcctttgcttgcctcagttttttttatctttcaactatatccattttgcaagctaatgaaacggatacgctacagttccggcgatttactttttcggatagagagattcagttcgtcctagatcccgcctctttaattaccgcccaaaacatcgttttatctaatttacagtcaaacggaaccggagcctgtaccatttcaggcaatacgcaaactcaaatcttttctaattccgttaacaccaccgcagattctggtggagcctttgatatggttactacctcattcacggcctctgataatgctaatctactcttctgcaacaactactgcacacataataaaggcggaggagctattcgttccggaggacctattcgattcttaaataatcaagacgtgcttttttataataacatatcggcaggggctaaatatgttggaacaggagatcacaacgaaaaaaataggggcggtgcgctttatgcaactactatcactttgacagggaatcgaactcttgcctttattaacaatatgtctggagactgcggtggagccatctctgctgacactcaaatatcaataactgataccgttaaaggaattttatttgaaaacaatcacacgctcaatcatataccgtacacgcaagctgaaaatatggcacgaggaggagcaatctgtagtagaagagacttgtgctcaatcagcaataattctggtcccatagtttttaactataaccaaggcgggaaaggtggagctattagcgctacccgatgtgttattgacaataacaaagaaagaatcatcttttcaaacaatagttccctgggatggagccaatcttcttctgcaagtaacggaggagccattcaaacgacacaaggatttactttacgaaataataaaggctctatctacttcgacagcaacactgctacacacgccgggggagccattaactgtggttacattgacatccgagataacggacccgtctattttctaaataactctgctgcctggggagcggcctttaatttatcgaaaccacgttcagcgacaaattatatccatacagggacaggcgatattgtttttaataataacgttgtctttactcttgacggtaatttattagggaaacggaaactttttcatattaataataatgagataacaccatatacattgtctctcggcgctaaaaaagatactcgtatctatttttatgatcttttccaatgggagcgtgttaaagaaaatactagcaataacccaccatctcctaccagtagaaacaccattaccgttaacccggaaacagagttttctggagctgttgtgttctcctacaatcaaatgtctagtgacatacgaactctgatgggtaaagaacacaattacattaaagaagccccaactactttaaaattcggaacgctagccatagaagatgatgcagaattagaaatcttcaatatcccgtttacccaaaatccgactagccttcttgctttaggaagcggcgctacgctgactgttggaaagcacggtaagctcaatattacaaatcttggtgttattttacccattattctcaaagaggggaagagtccgccttgtattcgcgtcaacccacaagatatgacccaaaatactggtaccggccaaactccatcaagcacaagtagtataagcactccaatgattatctttaatgggcgcctctcaattgtagacgaaaattatgaatcagtctacgacagtatggacctctccagagggaaagcagaacaactaattctatccatagaaaccactaatgatgggcaattagactccaattggcaaagttctctgaatacttctctactctctcctccacactatggctatcaaggtctatggactcctaattggataacaacaacctataccatcacgcttaataataattcttcagctccaacatctgctacctccatcgctgagcagaaaaaaactagtgaaacttttactcctagtaacacaactacagctagtatccctaatattaaagcttccgcaggatcaggctctggatcggcttccaattcaggagaagttacgattaccaaacatacccttgttgtaaactgggcaccagtcggctacatagtagatcctattcgtagaggagatctgatagccaatagcttagtacattcaggaagaaacatgaccatgggcttacgatcattactcccggataactcttggtttgctttgcaaggagctgcaacaacattatttacaaaacaacaaaaacgtttgagttatcatggctactcttctgcatcaaaggggtataccgtctcttctcaagcatcaggagctcatggtcataagtttcttctttccttctcccagtcatctgataagatgaaagaaaaagaaacaaataaccgcctttcttctcgttactatctttctgctttatgtttcgaacatcctatgtttgatcgcattgctcttatcggagcagcagcttgcaattatggaacacataacatgcggagtttctatggaactaaaaaatcttctaaagggaaatttcactctacaaccttaggagcttctcttcgctgtgaactacgcgatagtatgcctttacgatcaataatgctcaccccatttgctcaggctttattctctcgaacagaaccagcttctatccgagaaagcggtgatctagctagattatttacattagagcaagcccatactgccgttgtctctccaataggaatcaaaggagcttattcttctgatacatggccaacactctcttgggaaatggaactagcttaccaacccaccctctactggaaacgtcctctactcaacacactattaatccaaaataacggttcttgggtcaccacaaataccccattagctaaacattccttttatgggagaggttctcactccctcaaattttctcatctgaaactatttgctaactatcaagcagaagtggctacttccactgtctcacactacatcaatgcaggaggagctctggtcttttaa

SEQ ID NO: 9 - protein, 987 aa, Chlamydia muridarum
MMQTPFHKFFLLAMLSYSLLQGGHAADISMPPGIYDGTTLTAPFPYTVIGDPRGTKVTSSGSLELKNLDNSIATLPLSCFGNLLGNFTIAGRGHSLVFENIRTSTNGAALSNHAPSGLFVIEAFDELSLLNCNSLVSVVPQTGGTTTSVPSNGTIYSRTDLVLRDIKKVSFYSNLVSGDGGAIDAQSLMVNGIEKLCTFQENVAQSDGGACQVTKTFSAVGNKVPLSFLGNVAGNKGGGVAAVKDGQGAGGATDLSVNFANNTAVEFEGNSARIGGGIYSDGNISFLGNAKTVFLSNVASPIYVDPAAAGGQPPADKDNYGDGGAIFCKNDTNIGEVSFKDEGVVFFSKNIAAGKGGAIYAKKLTISDCGPVQFLGNVANDGGAIYLVDQGELSLSADRGDIIFDGNLKRMATQGAATVHDVMVASNAISMATGGQITTLRAKEGRRILFNDPIEMANGQPVIQTLTVNEGEGYTGDIVFAKGDNVLYSSIELSQGRIILREQTKLLVNSLTQTGGSVHMEGGSTLDFAVTTPPAANSMALTNVHFSLASLLKNNGVTNPPTNPPVQVSSPAVIGNTAAGTVTISGPIFFEDLDETAYDNNQWLGADQTIDVLQLHLGANPPANAPTDLTLGNESSKYGYQGSWTLQWEPDPANPPQNNSYMLKASWTKTGYNPGPERVASLVSNSLWGSILDVRSAHSAIQASIDGRAYCRGIWISGISNFFYHDQDALGQGYRHISGGYSIGANSYFGSSMFGLAFTETFGRSKDYVVCRSNDHTCVGSVYLSTRQALCGSCLFGDAFVRASYGFGNQHMKTSYTFAEESNVRWDNNCVVGEVGAGLPIMLAASKLYLNELRPFVQAEFAYAEHESFTERGDQAREFKSGHLMNLSIPVGVKFDRCSSKHPNKYSFMGAYICDAYRSISGTETTLLSHKETWTTDAFHLARHGVMVRGSMYASLTGNIEVYGHGKYEYRDASRGYGLSIGSKIRF

SEQ ID NO: 10 - protein, 1013 aa, Chlamydia trachomatis
MQTSFHKFFLSMILAYSCCSLSGGGYAAEIMIPQGIYDGETLTVSFPYTVIGDPSGTTVFSAGELTLKNLDNSIAALPLSCFGNLLGSFTVLGRGHSLTFENIRTSTNGAALSDSANSGLFTIEGFKELSFSNCNSLLAVLPAATTNNGSQTPTTTSTPSNGTIYSKTDLLLLNNEKFSFYSNLVSGDGGAIDAKSLTVQGISKLCVFQENTAQADGGACQVVTSFSAMANEAPIAFIANVAGVRGGGIAAVQDGQQGVSSSTSTEDPVVSFSRNTAVEFDGNVARVGGGIYSYGNVAFLNNGKTLFLNNVASPVYIAAEQPTNGQASNTSDNYGDGGAIFCKNGAQAAGSNNSGSVSFDGEGVVFFSSNVAAGKGGAIYAKKLSVANCGPVQFLGNIANDGGAIYLGESGELSLSADYGDIIFDGNLKRTAKENAADVNGVTVSSQAISMGSGGKITTLRAKAGHQILFNDPIEMANGNNQPAQSSEPLKINDGEGYTGDIVFANGNSTLYQNVTIEQGRIVLREKAKLSVNSLSQTGGSLYMEAGSTLDFVTPQPPQQPPAANQLITLSNLHLSLSSLLANNAVTNPPTNPPAQDSHPAIIGSTTAGSVTISGPIFFEDLDDTAYDRYDWLGSNQKIDVLKLQLGTQPSANAPSDLTLGNEMPKYGYQGSWKLAWDPNTANNGPYTLKATWTKTGYNPGPERVASLVPNSLWGSILDIRSAHSAIQASVDGRSYCRGLWVSGVSNFFYHDRDALGQGYRYISGGYSLGANSYFGSSMFGLAFTEVFGRSKDYVVCRSNHHACIGSVYLSTKQALCGSYLFGDAFIRASYGFGNQHMKTSYTFAEESDVRWDNNCLVGEIGVGLPIVITPSKLYLNELRPFVQAEFSYADHESFTEEGDQARAFRSGHLMNLSVPVGVKFDRCSSTHPNKYSFMGAYICDAYRTISGTQTTLLSHQETWTTDAFHLARHGVIVRGSMYASLTSNIEVYGHGRYEYRDTSRGYGLSAGSKVRF

SEQ ID NO: 11 - DNA, 2964 bp, Chlamydia muridarum
gtgatgcaaacgccttttcataagttctttcttctagcaatgctatcttactctttattgcaaggagggcatgcggcagatatttccatgcctccgggaatttatgatgggacaacattgacggcgccatttccctacactgtgatcggagatcccagagggacaaaggttacttcatcgggatcgctagagttgaaaaacctggacaattccattgcgactttacctctaagttgttttggtaatttgttggggaatttcactattgcaggaagagggcattcgttagtatttgagaatatacgaacatctacaaatggggcggcattgagtaatcatgctccttctggactgtttgtaattgaagcttttgatgaactctctcttttgaattgtaattcattggtatctgtagttcctcaaacagggggtacgactacttctgttccttctaatgggacgatctattctagaacagatcttgttctaagagatatcaagaaggtttctttctatagtaacttagtttctggagatgggggagctatagatgcacaaagtttaatggttaacggaattgaaaaactttgtaccttccaagaaaatgtagcgcagtccgatgggggagcgtgtcaggtaacaaagaccttctctgctgtgggcaataaggttcctttgtcttttttaggcaatgttgctggtaataaggggggaggagttgctgctgtcaaagatggtcagggggcaggaggggcgactgatctatcggttaattttgccaataatactgctgtagaatttgagggaaatagtgctcgaataggtggagggatctactcggacggaaatatttcctttttagggaatgcaaagacagttttcctaagtaacgtagcttcgcctatttatgttgaccctgctgctgcaggaggacagccccctgcagataaagataactatggagatggaggagccatcttctgcaaaaatgatactaacataggtgaagtctctttcaaagacgagggtgttgttttctttagtaaaaatattgccgcaggaaaggggggcgctatttatgctaagaaactgacaatttctgactgtggtccggtccagtttcttggtaatgtcgcgaatgacgggggcgctatttatctagtagatcagggggaacttagtctatctgctgatcgcggagatattatttttgatggaaatttaaagagaatggctacgcaaggcgctgccaccgtccatgatgtaatggttgcatcgaatgctatctctatggctacaggggggcaaatcacaacattaagggctaaggaaggtcgccgaattctttttaatgaccctattgaaatggcgaatggacaacctgtaatacaaactcttacagtaaacgagggcgaaggatatacgggggacattgtttttgctaaaggtgataatgttttgtactcaagtattgagctgagtcagggaagaattattctccgagagcaaacaaaattattggttaactccctgactcagactggagggagtgtacatatggaaggggggagtacactagactttgcagtaacaacgccaccagctgctaattcgatggctcttactaatgtacacttctccttagcttctttactaaaaaataatggggttacaaatcctccaacgaatcctccagtacaggtttctagtccagctgtaattggtaatacagctgctggtactgttacgatttctggtccgatcttttttgaagatttagatgaaactgcttacgataataatcagtggttaggtgcggatcaaactattgatgtgctgcagttgcatttaggagcgaatcctccggctaacgctccaactgatttgactttagggaacgaaagttctaaatatgggtatcaaggaagttggacacttcaatgggaaccagatcctgcgaatcctccacagaacaatagctacatgttgaaggcaagctggactaaaacaggttataatcctggtccggagcgcgtagcttctctggtctctaatagtctttggggatccattttagatgtgcgttccgcgcattctgcgattcaagcaagtatagatggacgagcttattgtcggggtatttggatttctgggatttcgaactttttctatcatgatcaggatgctttaggacaggggtatcgtcatattagtgggggatattcgataggagcaaactcttatttcgggtcttctatgtttggacttgcttttactgaaacttttggtaggtccaaagattatgtggtctgtcgatctaacgatcacacttgtgtaggctctgtttacttatccactagacaagcgttatgcggatcctgtttatttggagatgcttttgttcgggcgagttacggatttggaaatcagcatatgaagacctcttatacatttgctgaagagagtaatgtgcgttgggataataactgtgtagtgggagaagttggagctgggctccctatcatgctcgctgcatctaagctttatctaaatgagttgcgtcccttcgtgcaagcagagtttgcttatgcagagcatgaatcttttacagagagaggggatcaggctagggagtttaagagtgggcatcttatgaatctatctattccagttggggtgaagtttgatcgatgctctagtaaacatcctaacaagtatagttttatgggagcttatatctgtgatgcttaccggtccatttctggaacggagacaacactcctgtctcataaagagacttggacaacagatgctttccatttagcaaggcatggagttatggtcagaggatctatgtatgcttctttaacaggtaatatagaagtctatggccatggaaaatatgaatacagggatgcctctcgagggtatggtttaagtattggaagtaaaatccgattctaa

SEQ ID NO: 12 - DNA, 3042 bp, Chlamydia trachomatis
atgcaaacgtctttccataagttctttctttcaatgattctagcttattcttgctgctctttaagtgggggggggtatgcagcagaaatcatgattcctcaaggaatttacgatggggagacgttaactgtatcatttccctatactgttataggagatccgagtgggactactgttttttctgcaggagagttaacgttaaaaaatcttgacaattctattgcagctttgcctttaagttgttttgggaacttattagggagttttactgttttagggagaggacactcgttgactttcgagaacatacggacttctacaaatggagctgcactaagtgacagcgctaatagcgggttatttactattgagggttttaaagaattatctttttccaattgcaactcattacttgccgtactgcctgctgcaacgactaataatggtagccagactccgacgacaacatctacaccgtctaatggtactatttattctaaaacagatcttttgttactcaataatgagaagttctcattctatagtaatttagtctctggagatgggggagctatagatgctaagagcttaacggttcaaggaattagcaagctttgtgtcttccaagaaaatactgctcaagctgatgggggagcttgtcaagtagtcaccagtttctctgctatggctaacgaggctcctattgcctttatagcgaatgttgcaggagtaagagggggagggattgctgctgttcaggatgggcagcagggagtgtcatcatctacttcaacagaagatccagtagtaagtttttccagaaatactgcggtagagtttgatgggaacgtagcccgagtaggaggagggatttactcctacgggaacgttgctttcctgaataatggaaaaaccttgtttctcaacaatgttgcttctcctgtttacattgctgctgagcaaccaacaaatggacaggcttctaatacgagtgataattacggagatggaggagctatcttctgtaagaatggtgcgcaagcagcaggatccaataactctggatcagtttcctttgatggagagggagtagttttctttagtagcaatgtagctgctgggaaagggggagctatttatgccaaaaagctctcggttgctaactgtggccctgtacaattcttagggaatatcgctaatgatggtggagcgatttatttaggagaatctggagagctcagtttatctgctgattatggagatattattttcgatgggaatcttaaaagaacagccaaagagaatgctgccgatgttaatggcgtaactgtgtcctcacaagccatttcgatgggatcgggagggaaaataacgacattaagagctaaagcagggcatcagattctctttaatgatcccatcgagatggcaaacggaaataaccagccagcgcagtcttccgaacctctaaaaattaacgatggtgaaggatacacaggggatattgtttttgctaatggaaacagtactttgtaccaaaatgttacgatagagcaaggaaggattgttcttcgtgaaaaggcaaaattatcagtgaattctctaagtcagacaggtgggagtctgtatatggaagctgggagtacattggattttgtaactccacaaccaccacaacagcctcctgccgctaatcagttgatcacgctttccaatctgcatttgtctctttcttctttgttagcaaacaatgcagttacgaatcctcctaccaatcctccagcgcaagattctcatcctgcaatcattggtagcacaactgctggttctgttacaattagtgggcctatcttttttgaggatttggatgatacagcttatgataggtatgattggctaggttctaatcaaaaaatcgatgtcctgaaattacagttagggactcagccctcagctaatgccccatcagatttgactctagggaatgagatgcctaagtatggctatcaaggaagctggaagcttgcgtgggatcctaatacagcaaataatggtccttatactctgaaagctacatggactaaaactgggtataatcctgggcctgagcgagtagcttctttggttccaaatagtttatggggatccattttagatatacgatctgcgcattcagcaattcaagcaagtgtggatgggcgctcttattgtcgaggattatgggtttctggagtttcgaatttcttctatcatgaccgcgatgctttaggtcagggatatcggtatattagtgggggttattccttaggagcaaactcctactttggatcatcgatgtttggtctagcatttaccgaagtatttggtagatctaaagattatgtagtgtgtcgttccaatcatcatgcttgcataggatccgtttatctatctaccaaacaagctttatgtggatcctatttgttcggagatgcgtttatccgtgctagctacgggtttgggaaccagcatatgaaaacctcatacacatttgcagaggagagcgatgttcgttgggataataactgtctggttggagagattggagtgggattaccgattgtgattactccatctaagctctatttgaatgagttgcgtcctttcgtgcaagctgagttttcttatgccgatcatgaatcttttacagaggaaggcgatcaagctcgggcattcaggagtggacatctcatgaatctatcagttcctgttggagtaaaatttgatcgatgttctagtacacaccctaataaatatagctttatgggggcttatatctgtgatgcttatcgcaccatctctgggactcagacaacactcctatcccatcaagagacatggacaacagatgcctttcatttggcaagacatggagtcatagttagagggtctatgtatgcttctctaacaagcaatatagaagtatatggccatggaagatatgagtatcgagatacttctcgaggttatggtttgagtgcaggaagtaaagtccggttctaa

SEQ ID NO: 13 - protein, 983 aa, Chlamydia muridarum
MLVMPFSLRSTSFCFLACLCSYSYGLASSPQVLTPNVIIPFKGDDIYLNGDCVFASIYAGAEQGSIISANGQNLTIVGQNHTLSFTDSQGPALQNCAFISAEEKISLRDFSSLLFSKNVSCGEKGMISGKTVSISGGDSIVFKDNSVGYSSLPSVGQTPTTPIVGDVLKGSIFCVETGLEISGVKKELVFDNTAGNFGAVFCSRAAQGDTTFTVKDCKGKILFQDNVGSCGGGVIYKGEVLFQDNEGEMLFRGNSAHDDLGILDANPQPPTEVGGGGGVICTPEKTVTFKGNKGPITFDYNFAKGRGGAIQSQTFSLVADSAVVFSNNTAEKGGGAIYALEVNVSTNGGSILFEGNRASEGGAICVSEPIAANNGGLTLHAADGDIIFSKNMTSDRPGERSAIRILDSGTNVSLNASGASKMIFYDPVVQNNPATPPTGTSGEIKINESGSGSVVFTAETLTPSEKLNVINATSNFPGNLTVSSGELVVTKGATLTVGNITATSGRVTLGSGASLSAVAGTAGTCTVSKLGIDLESFLVPTYETAKLGADTTVAVNNNPTLDLVMANETEMYDNPLFMNAVTIPFVTLVSLQTTGGVTTSAVTLNNADTAHYGYQGSWSADWRRPPLAPDPSGMTPLDKSNTLYVTWRPSSNYGVYKLDPQRRGELVPNSLWVSGSALRTFTNGLKEHYVSRDVGFIASVQALGDYVLNYKQGNRDGFLARYGGFQAVAASHYENGGIFGVAFGQLYGQTKSRLYDSKDAGNITILSCFGRSYIDVKGTETVVYWETAYGYSVHRMHTQYFNGKTNKFDHSKCRWHNNSYYAFVGAEHNFLEYCIPTRQLARDYDLTGFMRFEMSGGWSSGAKETGALPRHFDRGTGHNMSLPIGVVAHAVSNGRRSPPSKLTINMGYRPDIWRVTPHCNMKIIANGVKTPIQGSPLARHAFFLEVHDTLYVRHLGRAYMNYSLDARHRQTTHFVSLGLNRIF

SEQ ID NO: 14 - protein, 1016 aa, Chlamydia trachomatis
MPFSLRSTSFCFLACLCSYSYGFASSPQVLTPNVTTPFKGDDVYLNGDCAFVNVYAGAENGSIISANGDNLTITGQNHTLSFTDSQGPVLQNYAFISAGETLTLKDFSSLMFSKNVSCGEKGMISGKTVSISGAGEVIFWDNSVGYSPLSIVPASTPTPPAPAPAPAASSSLSPTVSDARKGSIFSVETSLEISGVKKGVMFDNNAGNFGTVFRGNSNNNAGSGGSGSATTPSFTVKNCKGKVSFTDNVASCGGGVVYKGTVLFKDNEGGIFFRGNTAYDDLGILAATSRDQNTETGGGGGVICSPDDSVKFEGNKGSIVFDYNFAKGRGGSILTKEFSLVADDSVVFSNNTAEKGGGAIYAPTIDISTNGGSILFERNRAAEGGAICVSEASSGSTGNLTLSASDGDIVFSGNMTSDRPGERSAARILSDGTTVSLNASGLSKLIFYDPVVQNNSAAGASTPSPSSSSMPGAVTINQSGNGSVIFTAESLTPSEKLQVLNSTSNFPGALTVSGGELVVTEGATLTTGTITATSGRVTLGSGASLSAVAGAANNNYTCTVSKLGIDLESFLTPNYKTAILGADGTVTVNSGSTLDLVMESEAEVYDNPLFVGSLTIPFVTLSSSSASNGVTKNSVTINDADAAHYGYQGSWSADWTKPPLAPDAKGMVPPNTNNTLYLTWRPASNYGEYRLDPQRKGELVPNSLWVAGSALRTFTNGLKEHYVSRDVGFVASLHALGDYILNYTQDDRDGFLARYGGFQATAASHYENGSIFGVAFGQLYGQTKSRMYYSKDAGNMTMLSCFGRSYVDIKGTETVMYWETAYGYSVHRMHTQYFNDKTQKFDHSKCHWHNNNYYAFVGAEHNFLEYCIPTRQFARDYELTGFMRFEMAGGWSSSTRETGSLTRYFARGSGHNMSLPIGIVAHAVSHVRRSPPSKLTLNMGYRPDIWRVTPHCNMEIIANGVKTPIQGSPLARHAFFLEVHDTLYIHHFGRAYMNYSLDARRRQTAHFVSMGLNRIF

SEQ ID NO: 15 - DNA, 2952 bp, Chlamydia muridarum
gtgttagtaatgcctttttctttgagatctacatcattttgttttttagcctgtttatgttcttattcatatggattagcgagttctcctcaggtactgacccccaatgtaatcatcccttttaaaggagacgatatctatttaaatggggattgcgtttttgcaagtatctatgcaggagcagagcagggatcgattatttctgctaatgggcaaaatctaacaatcgtaggacaaaaccacactttatcatttacggattcccaagggccagcccttcaaaattgtgctttcatttcagcagaagaaaagatctctctaagagatttttcgagccttttgttttcgaaaaatgtttcttgcggggagaaaggaatgatttcagggaaaaccgtaagcatttcagggggagatagtatagtttttaaggataactctgttggttattcttcattaccctctgtggggcaaactcctacaactccaattgttggcgatgttttaaagggatccattttttgtgtggagacaggtttagagatttctggagtcaaaaaagagcttgttttcgataacactgctgggaattttggggcagtattctgtagtcgtgccgctcaaggagacacgactttcacagtgaaagactgtaagggtaaaattctttttcaagataacgtaggctcttgtggaggcggcgtaatttataaaggggaagtacttttccaagataatgaaggagaaatgcttttccgaggaaattcagctcatgatgatttgggaattctcgatgctaacccacagcctcctactgaagtaggaggtgggggtggtgtcatttgtaccccagagaaaacggtaacttttaaggggaataaagggcctattacctttgattataattttgcaaaaggtcgaggaggggcaatccaatcacagaccttttctttggtagctgatagtgctgttgttttcagtaataatacagctgagaaaggtggaggcgccatttatgctcttgaggttaacgtgagcacaaatggaggatctattctttttgagggaaatagagcttctgagggtggggctatctgtgtgagcgagccgatcgctgctaataatggagggctcactttacatgctgctgatggggacattattttctcgaaaaatatgacgagtgatcgtcctggagaacgcagtgcaatccggatcttagatagtggaacaaatgtctctttaaatgcttcaggggcatcgaagatgattttttatgatcctgttgtgcaaaataatcccgcaactccacctactggtacgtctggggaaattaagatcaatgagtccgggagtggatcggttgtgtttacagcagagactttgactccttcggaaaaattgaatgttatcaacgctacttctaatttcccaggaaatttaacggtatctagtggagaattagttgttacgaagggagcgacactaacagtaggaaatatcacagcaacatcaggacgagtaactttaggatcaggggcttcgttatccgccgttgcaggtactgctggcacttgtacggtgtctaaattagggattgatttagagtccttcctagtccctacttatgagactgcaaagttgggtgcggatacaacagtagcggtgaataacaatcctactttagacctagtaatggcgaatgagacggagatgtatgataatccgctttttatgaacgctgttacaatcccttttgtgacattggtttctctccaaactactggtggtgttactacaagtgccgttactctgaataatgcagatactgcgcattatgggtatcaaggatcttggtctgctgattggagaaggcctcctttagctcctgatcctagcggcatgacacctcttgataaaagtaatacattgtatgtgacatggaggccatcctctaactacggtgtgtataagttagatcctcaaagaaggggtgagttggtcccgaattctttatgggtatctggatctgccttaagaacctttacaaatggtttgaaggaacattacgtctctagagatgtcggatttattgcatctgtacaagccttaggggattatgttctgaattataagcagggtaaccgagatggctttctagctaggtacggaggttttcaagctgttgcggcttctcactatgaaaatggggggatctttggggtagctttcggtcaactttatggtcaaactaagagccgtttgtacgattctaaggatgctggaaacattacgattttgtcctgttttggacgaagttatatcgatgttaaaggaacagaaaccgttgtgtattgggagacggcttatggatattctgttcatagaatgcatacgcagtatttcaatggaaaaacgaataagtttgatcattcgaaatgtcgttggcacaacaatagttattatgcatttgtaggtgcagaacataatttcttggagtattgtattcctactcgtcaattagctagggattatgatcttacaggatttatgcgtttcgaaatgtcgggaggttggtcgagtggtgcaaaagaaacgggtgctttacctagacattttgatcgaggaacagggcataatatgtctcttccaataggggttgtagctcatgctgtttctaatggacgaagatctcctccatctaaattgacgattaacatgggatatagaccagacatttggcgggtgactccacattgcaatatgaaaattattgcaaacggagttaagactcctatacagggatctcctctagctcggcacgccttctttttagaagttcatgatactctgtatgttcgtcatttgggcagagcctatatgaattattctttagatgctcgtcatcgacaaactacgcatttcgtatctttaggattgaatcgtatcttttaa

SEQ ID NO: 16 - DNA, 3051 bp, Chlamydia trachomatis
atgcctttttctttgagatctacatcattttgttttttagcttgtttgtgttcctattcgtatggattcgcgagctctcctcaagtgttaacacctaatgtaaccactccttttaagggggacgatgtttacttgaatggagactgcgcttttgtcaatgtctatgcaggggcagagaacggctcaattatctcagctaatggcgacaatttaacgattaccggacaaaaccatacattatcatttacagattctcaagggccagttcttcaaaattatgccttcatttcagcaggagagacacttactctgaaagatttttcgagtttgatgttctcgaaaaatgtttcttgcggagaaaagggaatgatctcagggaaaaccgtgagtatttccggagcaggcgaagtgattttttgggataactctgtggggtattctcctttgtctattgtgccagcatcgactccaactcctccagcaccagcaccagctcctgctgcttcaagctctttatctccaacagttagtgatgctcggaaagggtctattttttctgtagagactagtttggagatctcaggcgtcaaaaaaggggtcatgttcgataataatgccgggaattttggaacagtttttcgaggtaatagtaataataatgctggtagtgggggtagtgggtctgctacaacaccaagttttacagttaaaaactgtaaagggaaagtttctttcacagataacgtagcctcctgtggaggcggagtagtctacaaaggaactgtgcttttcaaagacaatgaaggaggcatattcttccgagggaacacagcatacgatgatttagggattcttgctgctactagtcgggatcagaatacggagacaggaggcggtggaggagttatttgctctccagatgattctgtaaagtttgaaggcaataaaggttctattgtttttgattacaactttgcaaaaggcagaggcggaagcatcctaacgaaagaattctctcttgtagcagatgattcggttgtctttagtaacaatacagcagaaaaaggcggtggagctatttatgctcctactatcgatataagcacgaatggaggatcgattctatttgaaagaaaccgagctgcagaaggaggcgccatctgcgtgagtgaagcaagctctggttcaactggaaatcttactttaagcgcttctgatggggatattgttttttctgggaatatgacgagtgatcgtcctggagagcgcagcgcagcaagaatcttaagtgatggaacgactgtttctttaaatgcttccggactatcgaagctgatcttttatgatcctgtagtacaaaataattcagcagcgggtgcatcgacaccatcaccatcttcttcttctatgcctggtgctgtcacgattaatcagtccggtaatggatctgtgatttttaccgccgagtcattgactccttcagaaaaacttcaagttcttaactctacttctaacttcccaggagctctgactgtgtcaggaggggagttggttgtgacggaaggagctaccttaactactgggaccattacagccacctctggacgagtgactttaggatccggagcttcgttgtctgccgttgcaggtgctgcaaataataattatacttgtacagtatctaagttggggattgatttagaatcctttttaactcctaactataagacggccatactgggtgcggatggaacagttactgttaacagcggctctactttagacctagtgatggagagtgaggcagaggtatatgataatccgctttttgtgggatcgctgacaattccttttgttactctatcttctagtagtgctagtaacggagttacaaaaaattctgtcactattaatgatgcagacgctgcgcactatgggtatcaaggctcttggtctgcagattggacgaaaccgcctctggctcctgatgctaaggggatggtacctcctaataccaataacactctgtatctgacatggagacctgcttcgaattacggtgaatatcgactggatcctcagagaaagggagaactagtacccaactctctttgggtagcgggatctgcattaagaacctttactaatggtttgaaagaacactatgtttctagagatgttggatttgtagcatctctgcatgctctcggggattatattttgaattatacgcaagatgatcgggatggctttttagctagatatgggggattccaggcgaccgcagcctcccattatgaaaatgggtcaatatttggagtggcttttggacaactctatggtcagacaaagagcagaatgtattactctaaagatgctgggaacatgacgatgttgtcctgtttcggaagaagttacgtagatattaaaggaacagaaactgttatgtattgggagacggcttatggctattctgtgcacagaatgcatacgcagtattttaatgacaaaacgcagaagttcgatcattcgaaatgtcattggcacaacaataactattatgcgtttgtgggtgccgagcataatttcttagagtactgcattcctactcgtcagttcgctagagattatgagcttacagggtttatgcgttttgaaatggccggaggatggtccagttctacacgagaaactggctccctaactagatatttcgctcgcgggtcagggcataatatgtcgcttccaataggaattgtagctcatgcagtttctcatgtgcgaagatctcctccttctaaactgacactaaatatgggatatagaccagacatttggcgtgtcactccacattgcaatatggaaattattgctaacggagtgaagacacctatacaaggatctccgctggcacggcatgccttcttcttagaagtgcatgatactttgtatattcatcattttggaagagcctatatgaactattcgctggatgctcgtcgtcgacaaacggcacattttgtatccatgggcttgaatagaatcttttaa

SEQ ID NO: 17 - protein, 387 aa, Chlamydia muridarum
  misc_feature (305)..(305): Xaa can be any naturally occurring amino acid
MKKLLKSVLAFAVLGSASSLHALPVGNPAEPSLMIDGILWEGFGGDPCDPCTTWCDAISLRLGYYGDFVFDRVLKTDVNKQFEMGAAPTGDADLTTAPTPASRENPAYGKHMQDAEMFTNAAYMALNIWDRFDVFCTLGATSGYLKGNSAAFNLVGLFGRDETAVAADDIPNVSLSQAVVELYTDTAFAWSVGARAALWECGCATLGASFQYAQSKPKVEELNVLCNAAEFTINKPKGYVGQEFPLNIKAGTVSATDTKDASIDYHEWQASLALSYRLNMFTPYIGVKWSRASFDADTIRIAQPXLETSILXMTTWNPTISGSGIDVDTKITDTLQIVSLQLNKMKSRKSCGLAIGTTIVDADKYAVTVETRLIDERAAHVNAQFRF

SEQ ID NO: 18 - protein, 391 aa, Chlamydia trachomatis
MKKLLKSVLVFAALSSASSLQALPVGNPAEPSLMIDGILWEGFGGDPCDPCATWCDAISMRVGYYGDFVFDRVLKTDVNKEFQMGAKPTTDTGNSAAPSTLTARENPAYGRHMQDAEMFTNAACMALNIWDRFDVFCTLGATSGYLKGNSASFNLVGLFGDNENQKTVKAESVPNMSFDQSVVELYTDTTFAWSVGARAALWECGCATLGASFQYAQSKPKVEELNVLCNAAEFTINKPKGYVGKEFPLDLTAGTDAATGTKDASIDYHEWQASLLSYRLNMFTPYIGVKWSRASFDADTIRIAQPKSATAIFDTTTLNPTIAGAGDVKTGAEGQLGDTMQIVSLQLNKMKSRKSCGIAVGTTIVDADKYAVTVETRLIDERAAHVNAQFR

SEQ ID NO: 19 - DNA, 1164 bp, Chlamydia muridarum
atgaaaaaactcttgaaatcggtattagcatttgccgttttgggttctgcttcctccttgcatgctctgcctgtggggaatcctgctgaaccaagccttatgattgacgggattctttgggaaggtttcggtggagatccttgcgatccttgcacaacttggtgtgatgccatcagcctacgtctcggctactatggggacttcgtttttgatcgtgttttgaaaacagacgtgaacaaacagttcgaaatgggagcagctcctacaggagatgcagaccttactacagcacctactcctgcatcaagagagaatcccgcttatggcaagcatatgcaagatgcagaaatgttcactaatgctgcgtacatggctttaaacatttgggaccgtttcgatgtattttgtacattgggagcaactagcggatatcttaaaggtaattctgccgcctttaacttagttggtctgtttggaagagatgaaactgcagttgcagctgacgacatacctaacgtcagcttgtctcaagctgttgtcgaactctacacagacacagctttcgcttggagcgtcggtgctagagcagctttatgggagtgcggatgtgcaactttaggagcttccttccaatatgctcaatctaagccaaaagtagaggaattaaacgttctctgtaatgcggcagaattcactattaacaagcctaaaggatacgttggacaagagtttcctcttaacattaaagctggaacagttagcgctacagatactaaagatgcttccatcgattaccatgagtggcaagcaagcttggctttgtcttacagactgaatatgttcactccttacattggagttaagtggtctagagcaagctttgatgccgacactatccgcattgcgcagcctkagcttgagacctctatcttaakaatgaccacttggaacccaacgatctctggatctggtatagacgttgatacaaaaatcacggatacattacaaattgtttccttgcagctcaacaagatgaaatccagaaaatcttgcggtcttgcaattggaacaacaattgtagatgctgataaatatgcagttactgttgagacacgcttgatcgatgaaagagcagctcacgtaaatgctcagttccgtttctaa

SEQ ID NO: 20 - DNA, 1182 bp, Chlamydia trachomatis
atgaaaaaactcttgaaatcggtattagtatttgccgctttgagttctgcttcctccttgcaagctctgcctgtggggaatcctgctgaaccaagccttatgatcgacggaattctgtgggaaggtttcggcggagatccttgcgatccttgcgccacttggtgtgacgctatcagcatgcgtgttggttactacggagactttgttttcgaccgtgttttgaaaactgatgtgaataaagaatttcagatgggtgccaagcctacaactgatacaggcaatagtgcagctccatccactcttacagcaagagagaatcctgcttacggccgacatatgcaggatgctgagatgtttacaaatgccgcttgcatggcattgaatatttgggatcgttttgatgtattctgtacattaggagccaccagtggatatcttaaaggaaactctgcttctttcaatttagttggattgtttggagataatgaaaatcaaaaaacggtcaaagcggagtctgtaccaaatatgagctttgatcaatctgttgttgagttgtatacagatactacttttgcgtggagcgtcggcgctcgcgcagctttgtgggaatgtggatgtgcaactttaggagcttcattccaatatgctcaatctaaacctaaagtagaagaattaaacgttctctgcaatgcagcagagtttactattaataaacctaaagggtatgtaggtaaggagtttcctcttgatcttacagcaggaacagatgctgcgacaggaactaaggatgcctctattgattaccatgaatggcaagcaagtttagctctctcttacagactgaatatgttcactccctacattggagttaaatggtctcgagcaagctttgatgccgatacgattcgtatagcccagccaaaatcagctacagctatttttgatactaccacgcttaacccaactattgctggagctggcgatgtgaaaactggcgcagagggtcagctcggagacacaatgcaaatcgtttccttgcaattgaacaagatgaaatctagaaaatcttgcggtattgcagtaggaacaactattgtggatgcagacaaatacgcagttacagttgagactcgcttgatcgatgagagagcagctcacgtaaatgcacaattccgcttctaa

SEQ ID NO: 21 - protein, 238 aa, Chlamydia muridarum
MNISGSIKQKLLQFLKKQKSPELLATYLFYLEQSLHLSPVVFVRDKVIFKSAEDAIALLEADKKIWRETEIQISSGKPEVNEQTKRIYICPFTGKVFADNVYANPLDAVYDWLSSCPQNKERQAGVAVKRFLVSDDPEVIRAYIVPPKEPLIKTVYASAITGKLFHSLPTLLEDFKTSYLRPMTLEEVQNQNKFQLESSFLTLLQDALEEEKIAEFVESLADDTAFHEYISQWVDTEE

SEQ ID NO: 22 - protein, 238 aa, Chlamydia trachomatis
MNISGSIKQKLLQFLKKQKSPELLATYLFYLEQSLHLSPVVFVRDKIIFKSAEDAIQLLEADKKIWRETEIQISSGKPEVNEQTKRIYICPFTGKVFADNVYANPQDAIYDWLSSCPQNRERQSGVAVKRFLVSDDPEVIRAYIVPPKEPIIKTVYASAVTGKLFHSLPTLLEDFKTSYLRPMTLEEVQNQNKFQLESSFLTLLQDALEEEKIAEFVESLADDTAFHKYISQWVDTEE

SEQ ID NO: 23 - protein, 1007 aa, Chlamydia muridarum
MTTPISNSPSSIPTVTVSTTTASSGSLGTSTVSSTTTSTSVAQTATTTSSASTSIIQSSGENIQSTTGTPSPITSSVSTSAPSPKASATANKTSSAVSGKITSQETSEESETQATTSDGEVSSNYDDVDTPTNSSDSTVDSDYQDVETQYKTISNNGENTYETIGSHGEKNTHVQESHASGTGNPINNQQEAIRQLRSSTYTTSPRNENIFSPGPEGLPNMSLPSYSPTDKSSLLAFLSNPNTKAKMLEHSGHLVFIDTTRSSFIFVPNGNWDQVCSMKVQNGKTKEDLGLKDLEDMCAKFCTGYNKFSSDWGNRVDPLVSSKAGIESGGHLPSSVIINNKFRTCVAYGPWNPKENGPNYTPSAWRRGHRVDFGKIFDGTAPFNKINWGSSPTPGDDGISFSNETIGSEPFATPPSSPSQTPVINVNVNVGGTNVNIGDTNVSKGSGTPTSSQSVDMSTDTSDLDTSDIDTNNQTNGDINTNDNSNNVDGSLSDVDSRVEDDDGVSDTESTNGNDSGKTTSTEENGDPSGPDILAAVRKHLDTVYPGENGGSTEGPLPANQNLGNVIHDVEQNGSAKETIITPGDTGPTDSSSSVDADADVEDTSDTDSGIGDDDGVSDTESTNGNNSGKTTSTEENGDPSGPDILAAVRKHLDTVYPGENGGSTEGPLPANQNLGNVIHDVEQNGAAQETIITPGDTESTDTSSSVNANADLEDVSDADSGFGDDDGISDTESTNGNDSGKNTPVGDGGTPSGPDILAAVRKHLDTVYPGENGGSTERPLPANQNLGDIIHDVEQNGSAKETVVSPYRGGGGNTSSPIGLASLLPATPSTPLMTTPRTNGKAAASSLMIKGGETQAKLVKNGGNIPGETTLAELLPRLRGHLDKVFTSDGKFTNLNGPQLGAIIDQFRKETGSGGIIAHTDSVPGENGTASPLTGSSGEKVSLYDAAKNVTQALTSVTNKVTLAMQGQKLEGIINNNNTPSSIGQNLFAAARATTQSLSSLIGTVQ

SEQ ID NO: 24 - protein, 1005 aa, Chlamydia trachomatis
MTNSISGYQPTVTTSTSSTTSASGASGSLGASSVSTTANATVTQTANATNSAATSSIQTTGETVVNYTNSASAPNVTVSTSSSSTQATATSNKTSQAVAGKITSPDTSESSETSSTSSSDHIPSDYDDVGSNSGDISNNYDDVGSNNGDISSNYDDAAADYEPIRTTENIYESIGGSRTSGPENTSGGAAAALNSLRGSSYSNYDDAAADYEPIRTTENIYESIGGSRTSGPENTSGGAAAALNSLRGSSYSNYDDAAADYEPIRTTENIYESIGGSRTSGPENTSDGAAAAALNSLRGSSYTTGPRNEGVFGPGPEGLPDMSLPSYDPTNKTSLLTFLSNPHVKSKMLENSGHFVFIDTDRSSFILVPNGNWDQVCSIKVQNGKTKEDLDIKDLENMCAKFCTGFSKFSGDWDSLVEPMVSAKAGVASGGNLPNTVIINNKFKTCVAYGPWNSQEASSGYTPSAWRRGHRVDFGGIFEKANDFNKINWGTQAGPSSEDDGISFSNETPGAGPAAAPSPTPSSIPIINVNVNVGGTNVNIGDTNVNTTNTTPTTQSTDASTDTSDIDDINTNNQTDDINTTDKDSDGAGGVNGDISETESSSGDDSGSVSSSESDKNASVGNDGPAMKDILSAVRKHLDVVYPGENGGSTEGPLPANQTLGDVISDVENKGSAQDTKLSGNTGAGDDDPTTTAAVGNGAEEITLSDTDSGIGDDVSDTASSSGDESGGVSSPSSESNKNTAVGNDGPSGLDILAAVRKHLDKVYPGDNGGSTEGPLQANQTLGDIVQDMETTGTSQETVVSPWKGSTSSTESAGGSGSVQTLLPSPPPTPSTTTLRTGTGATTTSLMMGGPIKADIITTGGGGRIPGGGTLEKLLPRIRAHLDISFDAQGDLVSTEEPQLGSIVNKFRQETGSRGILAFVESAPGKPGSAQVLTGTGGDKGNLFQAAAAVTQALGNVAGKVNLAIQGQKLSSLVNDDGKGSVGRDLFQAAAQTTQVLSALIDTVG

SEQ ID NO: 25 - protein, 446 aa, Chlamydia muridarum
MRTLSISMLILALSCGENTCLCAADSPKAKVDASIGNGASFSPFTGEIKGNRVRLRLAPHTDSSIIKELSKGDCLAVLGESKDYYVVAAPEGVRGYVFRTFVLDNVIEGEKVNVRLEPSTSAPILARLSKGTVVKTLGAAQGKWVEIALPKQCVFYVAKNFVKNVGALELYNQKEGQKKIALDLLNSAMSFADAELQKKVEDIDLDAIYKKMNLAQAEEFKDVPGLQPLVQKALERVQEAFLAKSLEKGSHKTVESYKPVETQAQLQPQRQVIEEKNVSVVPEAPVLSQVEEPKSVLTSSSEVEPLQDVGPIKGSLLSHYIRKKGFVKTSPVVEGRESFERSLFEVWVNLQPEEIRNGLTMESFYRDEQKKKRVLTGELEVYPHIVKNNPGDYLLKNGEDVVAFVYATSIDLSKWLGKRVVLECVSRPNNHFAFPAYIVLSIKEGA

SEQ ID NO: 26 - protein, 433 aa, Chlamydia trachomatis
MLIFALSFGADACLCAADLSKAKVEASVGDRAAFSPFTGEIKGNRVRLRLAPHTDSFIIKELSKGDCLAVLGESKDYYVVAAPEGVRGYVFRTFVLDNVIEGEKVNVRLEPSTSAPILARLSKGTVVKTLGAAQGKWIEIALPKQCVFYVAKNFVKNVGALDLYNQKEGQKKLALDLLSSAMDFADAELQKKIEDIDLDAIYKKMNLAQSEEFKDVPGLQSLVQKALERVQEAFLAKSLEKSSVKVPEIRHKVLEEIAVVSPAVEETPVVTKTEEQKVTTVPVPAPAVVTEPAQDLSSVKGSLLSHYIRKKGFVKASPVIEGRESFERSLFAVWLSLQPEEIRHQLTMESFYRDEQKKKRVLTGELEVYPHIVKNNPGDYLLKNGEDVVAFVYATSIDLSKWLGKSVVLECVSRPNNHFAFPAYIVLSVKEGA

SEQ ID NO: 27 - protein, 183 aa, Chlamydia muridarum
MSRKARDPIVLPQGVEVSIQNDEISVKGPKGSLTQVLAKEVEIAVKGNEVFVSPAAHIIDRPGRMQGLYWALIANMVKGVHLGFEKRLEMIGVGFRASVQGSFLDLSIGVSHPTKMPIPTGLEVSVEKNTLISIKGINKQLVGEFAACVRAKRPPEPYKGKGIRYENEYVRRKAGKAAKTGKK

SEQ ID NO: 28 - protein, 183 aa, Chlamydia trachomatis
MSRKARDPIVLPQGVEVSIQNDEISVKGPKGSLTQVLAKEVEIAVKGNEVFVTPAAHVVDRPGRIQGLYWALIANMVKGVHTGFEKRLEMIGVGFRAAVQGSLLDLSIGVSHPTKMPIPTGLEVSVEKNTLISIKGINKQLVGEFAACVRAKRPPEPYKGKGIRYENEYVRRKAGKAAKTGKK

SEQ ID NO: 29 - protein, 116 aa, Chlamydia muridarum
MSNFQKEEQGQTGILHLQGKLDGVSSPAVQESISESLSNGMKNIILDCAALDYISSAGIRVLLQSYHQVGKNAGKIALTSISKTVEQTLYVTGFLSYFKVFDSINEALQALEKENS

SEQ ID NO: 30 - protein, 116 aa, Chlamydia trachomatis
MSNFQKEEQGQTGILHLQGKLDGVSSPAVQESISESLSNGMKNIILDCGDLDYISSAGIRVLLQSYHQVGKNAGKIALTSVSKTVEQTLYVTGFLSYFKVFDSVNEALQALAKENS

SEQ ID NO: 31 - protein, 279 aa, Chlamydia muridarum
MKKNFLKGVVPIPGLSTDEGTGVKDQNLWLNNATLNVRGDATVEDKVTGRDLNVTGPKIQTDVDLSVGRDVKGGRTELGETVLKGDFSIKCDMGQAPQFTNLSDPLSARDAITFDYYRDRSTQAYNCVTGYRVSVAGESFLDLHANNSRDVESFTPMYRNRFYWNGNDKQRLYLKSPGIYQVAFQILRNSGYHAGNDDPTVFLRLYTSEYEYTNLCTGDTRGFSQGNTTNTSLYSIFSIPSIGDEMPFVRVFTKIYIDIARTMINVIWFPFGSSYTEED

SEQ ID NO: 32 - protein, 280 aa, Chlamydia trachomatis
MKKPVFTGGAPIPGISTEEGTGVKDQNLWMRNATLKVEGDATIDDTLTSRDLKVTGPTIHTDLDLSVGGDVKGGRTVLGETVLEGDFNIKCNQGQVPQFTNLSDPLSARDAITFDYYRDRSTQAYNCATHRNGALVNGNRFIDLRLHNSEDSESYTPMYRNRFYWKDNDQKKLYLKSPGIYQVAFQIFRSGGYHSGNDDPTIFLRLYTSAYEYTNLCTGDTRGFNPGNTTNTSLYSIFSIPSIGNEHPFIQVFTKIHVNIAYSMINVIWFPFGSSYKEAD

SEQ ID NO: 33 - protein, 939 aa, Chlamydia muridarum
MKRSLLLLFFCSSSFLTSCSKSFQTIQDENPLTILTPALADQKIAKIICPNGLPLMIVSSPKASESGAALVVKTGNNADPLEFPGLAHFTEHCVFLGNEKYPQPSGFPAFLSTHGGIYNAFTYPDKTCFLFSVNNSDLDTALDQFVHLFIHPLFRQEDLNKEVHAVEQEFAMHPTKDSRRMHRIQQIIAPKEHPLKRFGCGNLSTLDTVTSQDMQTWFSTHYSPENMAAIVYTTAPLDVAVPYIAKIFSQIPKSSKYTPQTPFTKTQDTSSLNKLFINKAVEPSPQLAIYWHLYDAPASLQGWAQSLVSVLSSEKEHSLIALLKKEHLITHMEAGTYNTSLNTQDFEIIYKLTTKGEREYQKVLQLTFAFLDYVRDEQLPAYILPELHRINSLEYTYSTQTELFLTLTQMIPDFASEPLATYPYQSTIYPKYSHEDEQTFAALLADPQQARYILSATLPSSLEDADEFYDPIFDTIFYEKPLNFEPIKDYSSLGFTFPQPNKFIPQKVQLLSQKKQHEGFAFSPKLTYNQDAITLYTCEDSFYTVPKMAMELRIRSPQIQRADVRSLVLRDLYSLLANETLVKHYDEALRAGMDFSVSPGATGVDLSLFGYTETSPVLIDSLLSSLRDLPLDKSLFLYYKDQLSEQYQKGLISCPMRAGLNKLSGELLEGFFSLEEKQNALDIISYEEFEDFAHKMLTELSIEAFTLGTLSSQDLSNVLASLSHFSEASLPYDPPSYYPKRKPLFTTDFSFQYPLSGNGMLLLEQNEHPNQYEDSVATSMLLSWIHNLYYNDLRTNQQLGYMVGAKYQEFAETPCGLFYIRSNKSSPEELVNKTQLFLQKIATDPEASGLSEEIFEQLRETYIQSVLLPSATPSAMARKLFSIAFETEKHDFSRPDKKIAAARSMNYSYFKEYCKEFFNQKFGPEIQLLIYGSDSQQNE

SEQ ID NO: 34 - protein, 956 aa, Chlamydia trachomatis
MDNHPPVINDPTNDPKNMKRSLSLLLLCIPSFLTACSKSFQTIRDENPLTILTPALADQKIAKILCPNGLSLMIVSSPHAAESGAALVVKTGNNADPVEFPGLAHFTEHCVFLGNEKYPEPSGFPAFLSTHGGIYNAFTYPDKTCFLFSVNNADLDNALDQFVHLFIQPLFRQEDLNKEVHAVEQEFAMHPTKDSRRMHRIQQLIAPKNHPLKRFGCGNLSTLNSVTTQDMQTWFATHYSPENMAAIVYTTAPLDTAVPYIASLFSEIPISAQYTPQKPFPKTQDTTALNKLFINKAVEPSPQLAIYWHFYDAPQSLQGWAQSLISILSSEKENSLVALLKKEQLITEMEAELYSTSHNTQDFEILYKLTNKGEREYQRVLQLTFAFLDYVRHERLPAYSLPEIQKINSLEYTYSTQTELFSTLSRMVPNFTSEPLATYPYRSLVYPEYSQEDEQTFATFLADPQQARYILSATLPSSWENADEFYDPIFDDTFYEKPLDFTPIQDSSSLGFAFPNPNKFIPQTVQLLSQKKQHEGFAFSPQLTYDQNAITLYTCEDSFYTIPKMAMELRIRSPQIQRTDVRSLVLRDLYSLLANETLIKRYDDALKAGMTFAVSPGATGVDLSLLGYTETSPVLINALLSSLRDLPVEESLFLYYKDQLSEQYQKNLIACPIRAGLNKLYSQILVDTVSLEDKLNTLNTLSYEEFANFTNKLLQELAVESLALGTLSAQDLSNLLSTLSNFAEASSPYAAPSYYPQRKPLSSTKLSFQYPLSGNGMLLLEQNEDPHQYKDSVATSMLLSWIHNLYFSDLRTEQQLGYMVGSKYLEFAETPCGLFYIRSNNYSPEELVHRTQLFIQKIATDPESAGLSEEIFEQLRETYIQSILLPSSTPLAMAKKLFSIAFETKKQDFSRPDQKIAAARSMDYSYFKKYCEEFLSQKFGPEIQLLVYGANSSQEK

SEQ ID NO: 35 - protein, 877 aa, Chlamydia trachomatis
MSSYYLNFRPTTVSGEGLFKIKLANPGSDFKNQARPAIDMEELNSGLYVLRRLAVALEAGYLGVGSVVNPSNRIFPGGDWGVRRAAGGRTPAAGIISGSTIADIKQSTAKVLVTTITDSLNALIEDVPELPMTQVAGISSTLVLMATYQQKPSLDETDQKAIFGSAYIPADTSIKDVIKKEQEKELQEGKDRITAQLTAQGASNQVIEKSLADYEKYYVNEYFDTHVKEALWKHRASIGENIQEMLDQCLVLGLDVPDSLTKENINDANAKLVLQAWMEAFNNAMEVEPALGGSKEVIDSVLKMIPFAKPDANLSAEDISSIYTQAALPSPEVMDYYLTRQDAGVCKGEVVKAFQQATQNLQSVRSNVEEQIKELEVKKTSFLQAQASLESMLEGVKRLSDNKDFTSVRLTSVMECYAGLMALSQIAGVLEDEGLTLITKYVNQFLQLNNANTDQTLAHVISYMVAYCEVAESTMASTISDENTVIQKVRNKWTELTKQKFFSSFNSTMPSEEELKKNYITNKNSVYQANFGNFVDNVITRNLNLSDAVTKAQSMLTEFQGKATEYLSKFQQEINELNRTYDTLDPAKASFNTSTGSTPSLRAQAVDSWIDSTSLGSAFIHLILNTQIPKQENFLNPLIQEVNFNNVAANAVNDLLSITNNFSTSSVYYNLSSYLVESKEGENLFCGDFFEFIGALAKEHEYIVRDINSCYRAEAFGEALLARVEALAQGHKVTDAQANSMRTQANLYLSFIRIIVEQLAVLDSLLRSLNYEVEKKDNNYDKDKYKITGPTDWISTLASLEGYAANGFDNASPSGGLGPMHTLVQTDQHDYLTQSQTQQLNLQNQMTNIQQEWTLVSTSMQVLNGILSHLAAEIYSN

SEQ ID NO: 36 - protein, 1112 aa, Artificial Sequence
  a fusion protein of PmpE [(1)...(557)] and PmpF [(558)...(1112)]
REVPPSILLKPILNPYHMTGLFFPKVNLLGDTHNLTDYHLDNLKCILACLQRTPYEGAAFTVTDYLGFSDTQKDGIFCFKNLTPESGGVIGSPTQNTPTIKIHNTIGPVLFENNTCHRLWTQTDPENEGNKAREGGAIHAGDVYISNNQNLVGFIKNFAYVQGGAISANTFAYKENKSSFLCLNNSCIQTKTGGKGGAIYVSTSCSFENNNKDLLFIQNSGCAGGAIFSPTCSLIGNQGDIVFYSNHGFKNVDNATNESGDGGAIKVTTRLDITNNGSQIFFSDNISRNFGGAIHAPCLHLVGNGPTYFTNNIANHTGGAIYITGTETSKISADHHAIIFDNNISANATNADGSSSNTNPPHRNAITMDNSAGGIELGAGKSQNLIFYDPIQVTNAGVTVDFNKDASQTGCVVFSGATVLSADISQANLQTKTPATLTLSHGLLCIEDRAQLTVNNFTQTGGIVALGNGAVLSSYQHSTTDATQTPPTTTTTDASVTLNHIGLNLPSILKDGAEMPLLWVEPISTTQGNTTTYTSDTAASFSLNGATLSLIDEDGNSSETDTLKLPNLTFGGREIEFIVTPPSSIAAQYITYANVSNYRGNFTISSCTQDQWFSRGLSTTNSSGAFVESMTSFTAIDNADLFFCNNYCTHQGGGGAINATGLISFKNNQNILFYNNTTIGTQFTGVALRTERNRGGALYGSSIELINNHSLNFINNTSGDMGGAVSTIQNLVIKNTSGIVAFENNHTTDHIPNTFATILARGGAVGCQGACEISHNTGPVVFNSNYGGYGGAISTGGQCIFRDNKDKLIFINNSALGWHNTSAQGNGAVISAGGEFGLLNNKGPIYFENNNASYIAGAISCNNLNFQENGPIYFLNNSALYGGAFHLFASPAANYIHTGSGDIIFNNNTELSTTGMSAGLRKLFYIPGTTNNNPITLSLGAKKDTRIYFYDLFQWGGLKKANTPPENSPHTVTINPSDEFSGAVVFSYKNISSDLQAHMIASKTHNQIKDSPTTLKFGTMSIENGAEFEFFNGPLTQESTSLLALGQDSILTVGKDASLTITHLGIILPGLLNDQGTTAPRIRVNPQDMTQNTNSNQAPVSTENVATQKIFF

SEQ ID NO: 37 - protein, 1098 aa, Artificial Sequence
  a fusion protein of PmpG [(1)...(550)] and PmpH [(551)...(1098)]
ADISMPPGIYDGTTLTAPFPYTVIGDPRGTKVTSSGSLELKNLDNSIATLPLSCFGNLLGNFTIAGRGHSLVFENIRTSTNGAALSNHAPSGLFVIEAFDELSLLNCNSLVSVVPQTGGTTTSVPSNGTIYSRTDLVLRDIKKVSFYSNLVSGDGGAIDAQSLMVNGIEKLCTFQENVAQSDGGACQVTKTFSAVGNKVPLSFLGNVAGNKGGGVAAVKDGQGAGGATDLSVNFANNTAVEFEGNSARIGGGIYSDGNISFLGNAKTVFLSNVASPIYVDPAAAGGQPPADKDNYGDGGAIFCKNDTNIGEVSFKDEGVVFFSKNIAAGKGGAIYAKKLTISDCGPVQFLGNVANDGGAIYLVDQGELSLSADRGDIIFDGNLKRMATQGAATVHDVMVASNAISMATGGQITTLRAKEGRRILFNDPIEMANGQPVIQTLTVNEGEGYTGDIVFAKGDNVLYSSIELSQGRIILREQTKLLVNSLTQTGGSVHMEGGSTLDFAVTTPPAANSMALTNVHFSLASLLKNNGVTNPPTNPPVQVSSPAVIGSSPQVLTPNVIIPFKGDDIYLNGDCVFASIYAGAEQGSIISANGQNLTIVGQNHTLSFTDSQGPALQNCAFISAEEKISLRDFSSLLFSKNVSCGEKGMISGKTVSISGGDSIVFKDNSVGYSSLPSVGQTPTTPIVGDVLKGSIFCVETGLEISGVKKELVFDNTAGNFGAVFCSRAAQGDTTFTVKDCKGKILFQDNVGSCGGGVIYKGEVLFQDNEGEMLFRGNSAHDDLGILDANPQPPTEVGGGGGVICTPEKTVTFKGNKGPITFDYNFAKGRGGAIQSQTFSLVADSAVVFSNNTAEKGGGAIYALEVNVSTNGGSILFEGNRASEGGAICVSEPIAANNGGLTLHAADGDIIFSKNMTSDRPGERSAIRILDSGTNVSLNASGASKMIFYDPVVQNNPATPPTGTSGEIKINESGSGSVVFTAETLTPSEKLNVINATSNFPGNLTVSSGELVVTKGATLTVGNITATSGRVTLGSGASLSAVAGTAGTCTVSKLGIDLESFLVPTYETAKLGADTTVAVNNNPTLDLVMANETEMYDNP

SEQ ID NO: 38 - DNA, 3336 bp, Artificial Sequence
  a fusion protein of pmpE [(1)...(1671)] and pmpF [(1672)...(3336)]
cgagaagtccctccttcgattcttttaaagcctatactaaatccataccatatgaccgggttattttttcccaaggttaatttgcttggagacacacataatctcactgattaccatttggataatctaaaatgcattctggcttgcctacaaagaactccttatgaaggagctgctttcacagtaaccgattacttaggtttttcagatacacaaaaggatggtattttttgttttaaaaatcttactccagagagtggaggggttattggttccccaactcaaaacactcctactataaaaattcataatacaatcggccccgttcttttcgaaaataatacctgtcatagactgtggacacagaccgatcccgaaaatgaaggaaacaaagcacgcgaaggcggggcaattcatgctggggacgtttacataagcaataaccagaaccttgtcggattcataaagaactttgcttatgttcaaggtggagctattagtgctaatacttttgcctataaagaaaataaatcgagctttctttgcctaaataactcttgtatacaaactaagacgggagggaaaggtggtgctatttacgttagtacgagctgctctttcgagaacaataacaaggatctgcttttcatccaaaactccggctgtgcaggaggagctatcttctctccaacctgttctctaataggaaaccaaggagatattgttttttacagcaaccacggttttaaaaatgttgataatgcaactaacgaatctggggatggaggagctattaaagtaactacccgcttggacatcaccaataatggtagtcaaatctttttttctgataatatctcaagaaattttggaggagctattcatgctccttgtcttcatcttgttggtaatgggccaacctattttacaaacaatatagctaatcacacaggtggggctatttatataacaggaacagaaacctcaaagatttctgcagatcaccatgctattatttttgataataacatttctgcaaacgccaccaatgcggacggatctagcagcaacactaatcctcctcacagaaatgcgatcactatggacaattccgctggaggaatagaacttggtgcagggaagagccagaatcttattttctatgatcctattcaagtgacgaatgctggagttaccgtagacttcaataaggatgcctcccaaaccggatgtgtagttttctctggagcgactgtcctttctgcagatatttctcaggctaatttgcaaactaaaacacctgcaacgcttactctcagtcacggtcttctgtgtatcgaagatcgtgctcagctcacagtgaacaattttacacaaacaggagggattgtagccttaggaaatggagcagttttaagcagctaccaacacagcactacagacgccactcaaactccccctacaaccaccactacagatgcttccgtaactcttaatcacattggattaaatctcccctctattcttaaggatggagcagagatgcctctattatgggtagaacctataagcacaactcaaggtaacactacaacatatacgtcagataccgcggcttccttctcattaaatggagccacactctctctcattgatgaagatggaaattctagtgaaaccgatacactcaaacttccgaacttgacttttggtggtagagagattgaattcatagttactccgcctagctccattgctgctcaatacatcacttacgcaaatgtttctaattatagagggaactttactatttcaagttgtacgcaggatcaatggttttcgagaggtttaagcactacaaactctagtggagcttttgttgagtctatgacttctttcacagccattgacaatgcagacttgtttttttgtaacaattattgcacccatcagggaggagggggagctataaatgctacaggccttattagctttaaaaacaaccaaaacatattgttctataataatacaactattggaactcaatttacaggagtagcattaagaaccgaaaggaatcgcggaggggctttatacggatcaagcatcgagctaattaataatcatagcttaaattttatcaataacacttctggggatatgggaggagccgtatccacaatccaaaacctagttatcaaaaatacgtccggaatagttgcttttgaaaataaccatactactgatcacatacccaacacatttgctacaattcttgctcgaggaggagctgttggctgccaaggtgcctgcgaaatctcacacaatactggtccggtagtcttcaattccaactatggaggatacggaggagctatcagcaccgggggacagtgtatttttagagataataaggataagcttatttttataaataatagcgctttaggatggcataacactagtgctcaaggaaatggagcagttataagcgcaggaggagagtttggtcttctaaataataaaggccctatctactttgagaataataatgcctcatacatagcaggagctatttcctgcaacaaccttaattttcaagaaaatggtcctatctattttcttaataattcggctctgtatggaggagcttttcacctatttgcaagcccagctgcgaactatattcatactggctctggggatattatcttcaacaataatacagagctttcaactaccggaatgtcagcaggtttgcgaaaacttttttatattcctggaacaaccaacaataaccctatcaccctatctcttggtgctaagaaagatactcgcatctatttttatgatctttttcaatggggaggcttaaaaaaagctaatacaccccctgaaaatagcccgcacaccgttaccatcaatccttcggatgagttctctggcgctgttgtgttttcatacaaaaacatatccagtgatctacaagctcacatgattgccagtaaaactcataaccaaattaaagactcccccactaccttgaagtttgggactatgtccatagaaaatggcgcagagtttgaatttttcaatggccctcttactcaagaaagcactagccttcttgctttaggacaagattctattcttactgtagggaaagacgcttctctcactattacgcatcttggaatcattttgccaggtcttctcaatgaccaaggtactacagctccacgtattcgtgttaatccccaagatatgacacagaatacaaactctaaccaagctccagtaagcacagagaacgtggcaactcaaaagatctttttc

SEQ ID NO: 39 - DNA, 3294 bp, Artificial Sequence
  a fusion protein of pmpG [(1)...(1650)] and pmpH [(1651)...(3294)]
gcagatatttccatgcctccgggaatttatgatgggacaacattgacggcgccatttccctacactgtgatcggagatcccagagggacaaaggttacttcatcgggatcgctagagttgaaaaacctggacaattccattgcgactttacctctaagttgttttggtaatttgttggggaatttcactattgcaggaagagggcattcgttagtatttgagaatatacgaacatctacaaatggggcggcattgagtaatcatgctccttctggactgtttgtaattgaagcttttgatgaactctctcttttgaattgtaattcattggtatctgtagttcctcaaacagggggtacgactacttctgttccttctaatgggacgatctattctagaacagatcttgttctaagagatatcaagaaggtttctttctatagtaacttagtttctggagatgggggagctatagatgcacaaagtttaatggttaacggaattgaaaaactttgtaccttccaagaaaatgtagcgcagtccgatgggggagcgtgtcaggtaacaaagaccttctctgctgtgggcaataaggttcctttgtcttttttaggcaatgttgctggtaataaggggggaggagttgctgctgtcaaagatggtcagggggcaggaggggcgactgatctatcggttaattttgccaataatactgctgtagaatttgagggaaatagtgctcgaataggtggagggatctactcggacggaaatatttcctttttagggaatgcaaagacagttttcctaagtaacgtagcttcgcctatttatgttgaccctgctgctgcaggaggacagccccctgcagataaagataactatggagatggaggagccatcttctgcaaaaatgatactaacataggtgaagtctctttcaaagacgagggtgttgttttctttagtaaaaatattgccgcaggaaaggggggcgctatttatgctaagaaactgacaatttctgactgtggtccggtccagtttcttggtaatgtcgcgaatgacgggggcgctatttatctagtagatcagggggaacttagtctatctgctgatcgcggagatattatttttgatggaaatttaaagagaatggctacgcaaggcgctgccaccgtccatgatgtaatggttgcatcgaatgctatctctatggctacaggggggcaaatcacaacattaagggctaaggaaggtcgccgaattctttttaatgaccctattgaaatggcgaatggacaacctgtaatacaaactcttacagtaaacgagggcgaaggatatacgggggacattgtttttgctaaaggtgataatgttttgtactcaagtattgagctgagtcagggaagaattattctccgagagcaaacaaaattattggttaactccctgactcagactggagggagtgtacatatggaaggggggagtacactagactttgcagtaacaacgccaccagctgctaattcgatggctcttactaatgtacacttctccttagcttctttactaaaaaataatggggttacaaatcctccaacgaatcctccagtacaggtttctagtccagctgtaattggtagttctcctcaggtactgacccccaatgtaatcatcccttttaaaggagacgatatctatttaaatggggattgcgtttttgcaagtatctatgcaggagcagagcagggatcgattatttctgctaatgggcaaaatctaacaatcgtaggacaaaaccacactttatcatttacggattcccaagggccagcccttcaaaattgtgctttcatttcagcagaagaaaagatctctctaagagatttttcgagccttttgttttcgaaaaatgtttcttgcggggagaaaggaatgatttcagggaaaaccgtaagcatttcagggggagatagtatagtttttaaggataactctgttggttattcttcattaccctctgtggggcaaactcctacaactccaattgttggcgatgttttaaagggatccattttttgtgtggagacaggtttagagatttctggagtcaaaaaagagcttgttttcgataacactgctgggaattttggggcagtattctgtagtcgtgccgctcaaggagacacgactttcacagtgaaagactgtaagggtaaaattctttttcaagataacgtaggctcttgtggaggcggcgtaatttataaaggggaagtacttttccaagataatgaaggagaaatgcttttccgaggaaattcagctcatgatgatttgggaattctcgatgctaacccacagcctcctactgaagtaggaggtgggggtggtgtcatttgtaccccagagaaaacggtaacttttaaggggaataaagggcctattacctttgattataattttgcaaaaggtcgaggaggggcaatccaatcacagaccttttctttggtagctgatagtgctgttgttttcagtaataatacagctgagaaaggtggaggcgccatttatgctcttgaggttaacgtgagcacaaatggaggatctattctttttgagggaaatagagcttctgagggtggggctatctgtgtgagcgagccgatcgctgctaataatggagggctcactttacatgctgctgatggggacattattttctcgaaaaatatgacgagtgatcgtcctggagaacgcagtgcaatccggatcttagatagtggaacaaatgtctctttaaatgcttcaggggcatcgaagatgattttttatgatcctgttgtgcaaaataatcccgcaactccacctactggtacgtctggggaaattaagatcaatgagtccgggagtggatcggttgtgtttacagcagagactttgactccttcggaaaaattgaatgttatcaacgctacttctaatttcccaggaaatttaacggtatctagtggagaattagttgttacgaagggagcgacactaacagtaggaaatatcacagcaacatcaggacgagtaactttaggatcaggggcttcgttatccgccgttgcaggtactgctggcacttgtacggtgtctaaattagggattgatttagagtccttcctagtccctacttatgagactgcaaagttgggtgcggatacaacagtagcggtgaataacaatcctactttagacctagtaatggcgaatgagacggagatgtatgataatccg

SEQ ID NO: 40 - protein, 1386 aa, Artificial Sequence
  a fusion protein of PmpE [(1)...(680)] and PmpF [(681)...(1386)
REVPPSILLKPILNPYHMTGLFFPKVNLLGDTHNLTDYHLDNLKCILACLQRTPYEGAAFTVTDYLGFSDTQKDGIFCFKNLTPESGGVIGSPTQNTPTIKIHNTIGPVLFENNTCHRLWTQTDPENEGNKAREGGAIHAGDVYISNNQNLVGFIKNFAYVQGGAISANTFAYKENKSSFLCLNNSCIQTKTGGKGGAIYVSTSCSFENNNKDLLFIQNSGCAGGAIFSPTCSLIGNQGDIVFYSNHGFKNVDNATNESGDGGAIKVTTRLDITNNGSQIFFSNISRNFGGAIHAPCLHLVGNGPTYFTNNIANHTGGAIYITGTETSKISADHHAIIFDNNISANATNADGSSSNTNPPHRNAITMDNSAGGIELGAGKSQNLIFYDPIQVTNAGVTVDFNKDASQTGCVVFSGATVLSADISQANLQTKTPATLTLSHGLLCIEDRAQLTVNNFTQTGGIVALGNGAVLSSYQHSTTDATQTPPTTTTTDASVTLNHIGLNLPSILKDGAEMPLLWVEPISTTQGNTTTYTSDTAASFSLNGATLSLIDEDGNSPYENTDLSRALYAQPMLAISEASDNQLQSESMDFSKVNVPHYGWQGLWTWGWAKTENPTTTPPATITDPKKANQFHRTLLLTWLPAGYIPSPKHKSPLIANTLWGNILFATENLKNSSGQELLDRPFWSETDTLKLPNLTFGGREIEFIVTPPSSIAAQYITYANVSNYRGNFTISSCTQDQWFSRGLSTTNSSGAFVESMTSFTAIDNADLFFCNNYCTHQGGGGAINATGLISFKNNQNILFYNNTTIGTQFTGVALRTERNRGGALYGSSIELINNHSLNFINNTSGDMGGAVSTIQNLVIKNTSGIVAFENNHTTDHIPNTFATILARGGAVGCQGACEISHNTGPVVFNSNYGGYGGAISTGGQCIFRDNKDKLIFINNSALGWHNTSAQGNGAVISAGGEFGLLNNKGPIYFENNNASYIAGAISCNNLNFQENGPIYFLNNSALYGGAFHLFASPAANYIHTGSGDIIFNNNTELSTTGMSAGLRKLFYIPGTTNNNPITLSLGAKKDTRIYFYDLFQWGGLKKANTPPENSPHTVTINPSDEFSGAVVFSYKNISSDLQAHMIASKTHNQIKDSPTTLKFGTMSIENGAEFEFFNGPLTQESTSLLALGQDSILTVGKDASLTITHLGIILPGLLNDQGTTAPRIRVNPQDMTQNTNSNQAPVSTENVATQKIFFSGLVSLVDENYESVYDSCDLSRGKANQPILHIETTNDAQLSNDWKNTLNTSLYSLPHYGYQGLWTSNWMTTTRTVSLTNSTETQTANNSIQEQKNTSETFDSNSTTTAKIPSIRASTGGTTPLATTDVTVTRHSLVVSWTPIGYIAD

SEQ ID NO: 41 - protein, 1314 aa, Artificial Sequence
  a fusion protein of PmpG [(1)...(688)] and PmpH [(689)...(1314)]
ADISMPPGIYDGTTLTAPFPYTVIGDPRGTKVTSSGSLELKNLDNSIATLPLSCFGNLLGNFTIAGRGHSLVFENIRTSTNGAALSNHAPSGLFVIEAFDELSLLNCNSLVSVVPQTGGTTTSVPSNGTIYSRTDLVLRDIKKVSFYSNLVSGDGGAIDAQSLMVNGIEKLCTFQENVAQSDGGACQVTKTFSAVGNKVPLSFLGNVAGNKGGGVAAVKDGQGAGGATDLSVNFANNTAVEFEGNSARIGGGIYSDGNISFLGNAKTVFLSNVASPIYVDPAAAGGQPPADKDNYGDGGAIFCKNDTNIGEVSFKDEGVVFFSKNIAAGKGGAIYAKKLTISDCGPVQFLGNVANDGGAIYLVDQGELSLSADRGDIIFDGNLKRMATQGAATVHDVMVASNAISMATGGQITTLRAKEGRRILFNDPIEMANGQPVIQTLTVNEGEGYTGDIVFAKGDNVLYSSIELSQGRIILREQTKLLVNSLTQTGGSVHMEGGSTLDFAVTTPPAANSMALTNVHFSLASLLKNNGVTNPPTNPPVQVSSPAVIGNTAAGTVTISGPIFFEDLDETAYDNNQWLGADQTIDVLQLHLGANPPANAPTDLTLGNESSKYGYQGSWTLQWEPDPANPPQNNSYMLKASWTKTGYNPGPERVASLVSNSLWGSILDVRSAHSAIQASIDGRAYCRGSSPQVLTPNVIIPFKGDDIYLNGDCVFASIYAGAEQGSIISANGQNLTIVGQNHTLSFTDSQGPALQNCAFISAEEKISLRDFSSLLFSKNVSCGEKGMISGKTVSISGGDSIVFKDNSVGYSSLPSVGQTPTTPIVGDVLKGSIFCVETGLEISGVKKELVFDNTAGNFGAVFCSRAAQGDTTFTVKDCKGKILFQDNVGSCGGGVIYKGEVLFQDNEGEMLFRGNSAHDDLGILDANPQPPTEVGGGGGVICTPEKTVTFKGNKGPITFDYNFAKGRGGAIQSQTFSLVADSAVVFSNNTAEKGGGAIYALEVNVSTNGGSILFEGNRASEGGAICVSEPIAANNGGLTLHAADGDIIFSKNMTSDRPGERSAIRILDSGTNVSLNASGASKMIFYDPVVQNNPATPPTGTSGEIKINESGSGSVVFTAETLTPSEKLNVINATSNFPGNLTVSSGELVVTKGATLTVGNITATSGRVTLGSGASLSAVAGTAGTCTVSKLGIDLESFLVPTYETAKLGADTTVAVNNNPTLDLVMANETEMYDNPLFMNAVTIPFVTLVSLQTTGGVTTSAVTLNNADTAHYGYQGSWSADWRRPPLAPDPSGMTPLDKSNTLYVTWRPSSNY

SEQ ID NO: 42 - protein, 16 aa, Chlamydia muridarum
AFHLFASPAANYIHTG

SEQ ID NO: 43 - protein, 19 aa, Chlamydia muridarum
NAKTVFLSNVASPIYVDPA

SEQ ID NO: 44 - protein, 17 aa, Chlamydia muridarum
ASPIYVDPAAAGGQPPA

SEQ ID NO: 45 - protein, 19 aa, Chlamydia muridarum
VKGNEVFVSPAAHIIDRPG

SEQ ID NO: 46 - protein, 18 aa, Chlamydia muridarum
SPGQTNYAAAKAGIIGFS

SEQ ID NO: 47 - protein, 16 aa, Chlamydia muridarum
KLDGVSSPAVQESISE

SEQ ID NO: 48 - protein, 15 aa, Chlamydia muridarum
IGQEITEPLANTVIA

SEQ ID NO: 49 - protein, 15 aa, Chlamydia muridarum
MTTVHAATATQSVVD

SEQ ID NO: 50 - protein, 14 aa, Artificial Sequence
  hypothetical protein
DLNVTGPKIQTDVD

SEQ ID NO: 51 - protein, 19 aa, Chlamydia muridarum PdhC TC0518
EGTKIPIGTPIAVFSTEQN

SEQ ID NO: 52 - protein, 17 aa, Chlamydia muridarum
SVPSYVYYPSGNRAPVV

SEQ ID NO: 53 - protein, 15 aa, Chlamydia muridarum
YDHIIVTPGANADIL

SEQ ID NO: 54 - protein, 17 aa, Chlamydia muridarum
LPLMIVSSPKASESGAA

SEQ ID NO: 55 - protein, 16 aa, Chlamydia muridarum
GANAIPVHCPIGAESQ

SEQ ID NO: 56 - protein, 15 aa, Chlamydia muridarum
VFWLGSKINIIDTPG

SEQ ID NO: 57 - protein, 16 aa, Chlamydia muridarum
ISRALYTPVNSNQSVG

SEQ ID NO: 58 - protein, 16 aa, Chlamydia muridarum
FEVQLISPVALEEGMR

SEQ ID NO: 59 - protein, 14 aa, Chlamydia muridarum
GDAAYIEKVRELMQ

SEQ ID NO: 60 - protein, 15 aa, Chlamydia muridarum
SRALYAQPMLAISEA

SEQ ID NO: 61 - protein, 17 aa, Chlamydia muridarum
KPAEEEAGSIVHNAREQ

SEQ ID NO: 62 - protein, 17 aa, Chlamydia muridarum
SPQVLTPNVIIPFKGDD

SEQ ID NO: 63 - protein, 10 aa, Chlamydia muridarum
SMLIIPALGG

SEQ ID NO: 64 - protein, 17 aa, Chlamydia muridarum
LAAAVMHADSGAILKEK

SEQ ID NO: 65 - protein, 16 aa, Artificial Sequence
  hypothetical protein
DDPEVIRAYIVPPKEP

SEQ ID NO: 66 - protein, 17 aa, Chlamydia muridarum
KIFSPAGLLSAFAKNGA

SEQ ID NO: 67 - protein, 15 aa, Chlamydia muridarum
DPVDMFQMTKIVSKH

SEQ ID NO: 68 - protein, 13 aa, Chlamydia muridarum
KLEGIINNNNTPS

SEQ ID NO: 69 - protein, 9 aa, Chlamydia muridarum
AVPRTSLIF

SEQ ID NO: 70 - protein, 17 aa, Chlamydia muridarum
GGAEVILSRSHPEFVKQ

SEQ ID NO: 71 - protein, 8 aa, Artificial Sequence
  hypothetical protein
APILARLS

(19) United States
(12) Patent Application Publication
(10) Pub. No.: US 2016/0045588 A1
(43) Pub. Date: Feb. 18, 2016

(54) CHLAMYDIA ANTIGEN COMPOSITIONS AND USES THEREOF

(71) Applicant: THE UNIVERSITY OF BRITISH COLUMBIA, Vancouver (CA)

(72) Inventors: Robert C. BRUNHAM, Sidney (CA); Karuna P. KARUNAKARAN, Richmond (CA); Hong YU, Vancouver (CA)

(21) Appl. No.: 14/616,510

(22) Filed: Feb. 6, 2015

Related U.S. Application Data
(63) Continuation-in-part of application No. PCT/CA2013/050610, filed on Aug. 8, 2013.
Application No. PCT/CA2013/050610 is a continuation-in-part of application No. 13/147,092, filed on Oct. 17, 2011.
(60) Provisional application No. 61/680,836, filed on Aug. 8, 2012.
(60) Provisional application No. 61/202,943, filed on Apr. 22, 2009.
(60) Provisional application No. 61/202,104, filed on Jan. 29, 2009.

(51) Int. Cl. A61K 39/118; C07K 14/295

(52) U.S. Cl. CPC A61K 39/118; C07K 14/295; A61K 2039/55555

(57) ABSTRACT

The present invention provides in part fusion proteins derived from Chlamydia spp. The present invention also provides in part methods for treating or preventing Chlamydia infection using the fusion proteins.

### FIELD OF INVENTION

The present invention relates to bacterial infections. More specifically, the invention provides in part fusion proteins for use against Chlamydia infection.

### BACKGROUND OF THE INVENTION

Chlamydia trachomatis is an intracellular pathogen responsible for over 92 million sexually transmitted infections and 85 million ocular infections per year worldwide (Starnbach, M. N., and N. R. Roan. 2008. Conquering sexually transmitted diseases. Nat Rev Immunol 8:313-317.). Sexually transmitted C. trachomatis is a major cause of long-term disease sequelae in women such as infertility and ectopic pregnancy (Brunham, R. C., D. J. Zhang, X. Yang, and G. M. McClarty. 2000. The potential for vaccine development against chlamydial infection and disease. J Infect Dis 181 Suppl 3:S538-543; Igietseme, J. U., C. M. Black, and H. D. Caldwell. 2002. Chlamydia vaccines: strategies and status. BioDrugs 16:19-35). C. trachomatis infection in women often goes unnoticed until severe reproductive damage (infertility, pelvic inflammatory disease, ectopic pregnancy) is already underway. In addition, women infected with C. trachomatis are at increased risk of contracting HIV following exposure.

The “seek and treat” programs to prevent and control C. trachomatis sexually transmitted infections appear to be failing as case rates and reinfection rates continue to rise (Brunham, R. C., B. Pourbohloul, S. Mak, R. White, and M. L. Rekart. 2005. The unexpected impact of a Chlamydia trachomatis infection control program on susceptibility to reinfection. J Infect Dis 192:1836-1844), possibly due to early treatment interfering with the development of protective immune responses (Su, H., R. Morrison, R. Messer, W. Whitmire, S. Hughes, and H. D. Caldwell. 1999. The effect of doxycycline treatment on the development of protective immunity in a murine model of chlamydial genital infection. J Infect Dis 180:1252-1258).

Previous attempts to vaccinate against C. trachomatis and C. muridarum infection in both human and murine models using dead elementary bodies (EBs), which are non-replicating infectious particles released when infected cells rupture, provided limited protection (Grayston, J. T., and S. P. Wang. 1978. The potential for vaccine against infection of the genital tract with Chlamydia trachomatis. Sex Transm Dis 5:73-77; Grayston, J. T., S. P. Wang, L. J. Yeh, and C. C. Kuo. 1985. Importance of reinfection in the pathogenesis of trachoma. Rev Infect Dis 7:717-725; Lu, H., Z. Xing, and R. C. Brunham. 2002. GM-CSF transgene-based adjuvant allows the establishment of protective mucosal immunity following vaccination with inactivated Chlamydia trachomatis. J Immunol 169:6324-6331; Schachter, J., and H. D. Caldwell. 1980. Chlamydiae. Annu Rev Microbiol 34:285-309). Mice immunized with live C. muridarum EBs have however been shown to generate better protection (Lu, H., Z. Xing, and R. C. Brunham. 2002. GM-CSF transgene-based adjuvant allows the establishment of protective mucosal immunity following vaccination with inactivated Chlamydia trachomatis. J Immunol 169:6324-6331; Su, H., R. Messer, W. Whitmire, E. Fischer, J. C. Portis, and H. D. Caldwell. 1998. Vaccination against chlamydial genital tract infection after immunization with dendritic cells pulsed ex vivo with nonviable Chlamydiae. J Exp Med 188:809-818).

Investigation into the mechanism underlying the efficient induction of immunity provided by live C. muridarum in comparison to dead organisms suggests that dendritic cells (DCs) exposed to live or dead C. muridarum develop into distinct phenotypes. In particular DCs exposed to live C. muridarum become mature and stimulated antigen-specific CD4 T cells, while DCs exposed to dead C. muridarum are inhibited in acquiring a mature phenotype. Co-stimulation of DCs with dead EB and CpG oligodeoxynucleotide has been show to partially overcome dead EB inhibition of DC maturation (Rey-Ladino, J., K. M. Koochesfahani, M. L. Zaharik, C. Shen, and R. C. Brunham. 2005. A live and inactivated Chlamydia trachomatis mouse pneumonitis strain induces the maturation of dendritic cells that are phenotypically and immunologically distinct. Infect Immun 73:1568-1577). Investigation into the transcriptional responses of bone marrow derived DCs following exposure to live and dead C. muridarum using GeneChip microarrays revealed marked differences in CXC chemokine profiles in DCs exposed to live or dead organism (Zaharik, M. L., T. Nayar, R. White, C. Ma, B. A. Vallance, N. Straka, X. Jiang, J. Rey-Ladino, C. Shen, and R. C. Brunham. 2007. Genetic profiling of dendritic cells exposed to live- or ultraviolet-irradiated Chlamydia muridarum reveals marked differences in CXC chemokine profiles. Immunology 120:160-172). In aggregate, the data suggest that DCs exposed to live EBs are phenotypically and functionally distinct from DCs generated by exposure to dead EBs.

Immunity to C. muridarum infection is thought to be largely cell-mediated and therefore dependent on Chlamydia-derived peptides presented to CD4 T cells via MHC molecules on antigen presenting cells (Brunham, R. C., and J. Rey-Ladino. 2005. Immunology of Chlamydia infection: implications for a Chlamydia trachomatis vaccine. Nat Rev Immunol 5:149-161; Steinman, R. M., and M. Pope. 2002. Exploiting dendritic cells to improve vaccine efficacy. J Clin Invest 109:1519-1526; Su, H., and H. D. Caldwell. 1995. CD4+ T cells play a significant role in adoptive immunity to Chlamydia trachomatis infection of the mouse genital tract. Infect Immun 63:3302-3308; Morrison, S. G., H. Su, H. D. Caldwell, and R. P. Morrison. 2000. Immunity to murine Chlamydia trachomatis genital tract reinfection involves B cells and CD4(+) T cells but not CD8(+) T cells. Infect Immun 68:6979-6987; Morrison, R. P., and H. D. Caldwell. 2002. Immunity to murine chlamydial genital infection. Infect Immun 70:2741-2751; Igietseme, J. U., K. H. Ramsey, D. M. Magee, D. M. Williams, T. J. Kincy, and R. G. Rank. 1993. Resolution of murine chlamydial genital infection by the adoptive transfer of a biovar-specific, Th1 lymphocyte clone. Reg Immunol 5:317-324).

Immunoproteomic approaches (Hunt, D. F., R. A. Henderson, J. Shabanowitz, K. Sakaguchi, H. Michel, N. Sevilir, A. L. Cox, E. Appella, and V. H. Engelhard. 1992. Characterization of peptides bound to the class I MHC molecule HLA-A2.1 by mass spectrometry. Science 255:1261-1263; de Jong, A. 1998. Contribution of mass spectrometry to contemporary immunology. Mass Spectrom Rev 17:311-335; Olsen, J. V., L. M. de Godoy, G. Li, B. Macek, P. Mortensen, R. Pesch, A. Makarov, O. Lange, S. Horning, and M. Mann. 2005. Parts per million mass accuracy on an Orbitrap mass spectrometer via lock mass injection into a C-trap. Mol Cell Proteomics 4:2010-2021) to identify C. muridarum T cell antigens, based on isolating and sequencing of pathogen-derived peptides binding to MHC class II molecules presented on the surface of DCs after they were pulsed with live EBs, resulted in the identification of a number of C. muridarum peptides derived from 8 novel epitopes (Karunakaran, K. P., J. Rey-Ladino, N. Stoynov, K. Berg, C. Shen, X. Jiang, B. R. Gabel, H. Yu, L. J. Foster, and R. C. Brunham. 2008. Immunoproteomic discovery of novel T cell antigens from the obligate intracellular pathogen Chlamydia. J Immunol 180:2459-2465). These peptides were recognized by antigen-specific CD4 T cells in vitro and recombinant proteins containing the MHC binding peptides were able to induce partial protection via immunization against C. muridarum infection in vivo (Yu, H., X. Jiang, C. Shen, K. P. Karunakaran, and R. C. Brunham. 2009. Novel Chlamydia muridarum T cell antigens induce protective immunity against lung and genital tract infection in murine models. J Immunol 182:1602-1608).

Chlamydia sequences (nucleic acid and polypeptide) are described in, for example, U.S. Pat. No. 6,030,799, U.S. Pat. No. 6,696,421, U.S. Pat. No. 6,676,949, U.S. Pat. No. 6,464,979, U.S. Pat. No. 6,653,461, U.S. Pat. No. 6,642,023, U.S. Pat. No. 6,887,843 and U.S. Pat. No. 7,459,524; or in US Patent Publications 2005/0232941, 2009/0022755, and 2008/0102112. Specific Chlamydia antigens are described in, for example, PCT Publication No. WO 2010/085896 and WO2013/044398.

### SUMMARY OF THE INVENTION

The present disclosure provides in part fusion proteins derived from Chlamydia spp. The present invention also provides in part methods for treating or preventing Chlamydia infection using the fusion proteins.

In one aspect, there is provided an immunogenic composition including a fusion protein which includes at least two Chlamydia proteins selected from: Polymorphic membrane protein G (PmpG), Polymorphic membrane protein F (PmpF), Polymorphic membrane protein E (PmpE), Polymorphic membrane protein H (PmpH), Ribosomal protein L6 (RplF), Anti-anti-sigma factor (Aasf), Translocated actin-recruiting phosphoprotein (Tarp), hypothetical protein corresponding to locus tag CT143/TC0420, metalloprotease, insulinase family (CT806/TC0190), hypothetical protein corresponding to locus tag CT538/TC0825, hypothetical protein corresponding to locus tag CT017/TC0285, hypothetical protein corresponding to locus tag CT619, or MOMP, or an immunogenic fragment thereof, together with a physiologically acceptable carrier.

In alternative embodiments, the fusion protein includes combinations of: PmpG and MOMP, PmpG and PmpF, PmpG and PmpH or PmpE and PmpF.

In alternative embodiments, the composition further includes an adjuvant, such as DDA/TDB, DDA/MMG or DDA/MPL.

In alternative aspects, there is provided a method for eliciting an immune response against a Chlamydia spp., or component thereof, in an animal by administering to the animal an effective amount of the composition as described herein, thereby eliciting an immune response in the animal. The immune response may be a cellular immune response.

In alternative aspects, there is provided a method for treating or preventing infection by a Chlamydia spp. in an animal by administering to the animal an effective amount of the composition as described herein, thereby treating or preventing infection by the Chlamydia spp. in the animal.

In alternative embodiments, the Chlamydia spp. may be a Chlamydia trachomatis or a Chlamydia muridarum.

In alternative embodiments, the animal may be a human.

In alternative aspects, there is provided the use of the composition as described herein, for eliciting an immune response against a Chlamydia spp., or component thereof, in an animal. The immune response may be a cellular immune response.

In alternative aspects, there is provided the use of the composition as described herein, for treating or preventing infection by a Chlamydia spp. or component thereof, in an animal. The Chlamydia spp. may be a Chlamydia trachomatis or a Chlamydia muridarum. The animal may be a human.

This summary does not necessarily describe all features of the invention.

### BRIEF DESCRIPTION OF THE DRAWINGS

These and other features of the disclosure will become more apparent from the following description in which reference is made to the appended drawings wherein:

FIGS. 1A-II show amino acid sequences for Chlamydia muridarum and Chlamydia trachomatis proteins, together with the corresponding nucleic acid sequences (SEQ ID Nos: 1-35).

FIGS. 2A-F show amino acid and nucleic acid sequences for PmpE-PmpF & PmpG-PmpH fusion proteins (italized sequences indicating the second protein; underlined sequences representing alpha helix linkers connecting two protein domains; SEQ ID NOs: 36-41).

FIGS. 3A-B are graphs showing fusion protein-elicited protection at day 6 (A) and day 12 (B) against Chlamydia genital tract infection.

FIGS. 4A-C are graphs showing C. muridarum-specific cytokine responses after immunization with PmpE, F, G, H plus MOMP either as individual (mixed) or as fusion formats in C57 (A), Balb/c (B), or C3H (C) mice.

FIGS. 5A-C are graphs showing C. muridarum individual antigen-specific IFN-γ responses in C57 (A), Balb/c (B), or C3H (C) mice after immunization with PmpE, F, G, H plus MOMP either as individual (mixed) or as fusion formats.

FIGS. 6A-L are graphs showing vaccine-elicited protection against C. muridarum genital tract infection in C57 (A-D), Balb/c (E-H), or C3H (I-L) mice after immunization with PmpE, F, G, H plus MOMP, either as individual (mixed) or as fusion formats.

FIGS. 7A-C are graphs showing vaccine-elicited protection against C. muridarum genital tract infection in C57 (A), Balb/c (B), or C3H (C) mice after immunization with PmpE, F, G, H plus MOMP, either as individual (mixed) or as fusion formats.

### DETAILED DESCRIPTION

The present disclosure provides, in part, fusion proteins derived from Chlamydia spp. proteins. The present disclosure also provides, in part, methods for treating or preventing Chlamydia infection using the fusion proteins.

In some embodiments, these fusion proteins may be useful as vaccines for use in the prevention or treatment of Chlamydia spp. infection.

Chlamydia Spp.

By “Chlamydia spp.” is meant a genus of bacteria that are obligate intracellular parasites. Chlamydia spp. include C. trachomatis (a human pathogen) and C. muridarum (pathogenic to mice and hamsters). As C. muridarum and C. trachomatis are highly orthologous pathogenic microbes that have co-evolved with their host species, C. muridarum has been used as a robust animal model for studying cellular immunity and vaccine development.

In some embodiments, a C. trachomatis includes without limitation a C. trachomatis serovar D/UW-3/CX, as well as serovars A, B, Ba, C (implicated in trachoma), serovars D, E, F, G, H, I, J K (implicated in urogenital tract infections) and L1, L2, L3 (lymphogranuloma venereum serovars).

In some embodiments, a C. muridarum includes a C. muridarum mouse pneumonitis (MoPn) strain Nigg.

The genome sequences of various Chlamydia spp. have been determined. The genome sequence of C. trachomatis strain D/UW-3/CX is described for example in Stephens, R. S. et al., 1998 (Genome sequence of an obligate intracellular pathogen of humans: Chlamydia trachomatis. Science 282 (5389): 754-759) and provided in GenBank Accession No. NC_{—}000117.1, GI:15604717; referred to herein as the “C. trachomatis genome sequence”).

The genome sequence of C. muridarum is described in for example Read, T., et al., 2000 (Genome sequences of Chlamydia trachomatis MoPn and Chlamydia pneumoniae AR39 Nucleic Acids Res. 28 (6): 1397-1406) and provided in GenBank Accession No. NC_{—}002620.2, GI:29337300; referred to herein as the “C. muridarum genome sequence”).

Chlamydia Spp. Fusion Polypeptides and Nucleic Acid Molecules

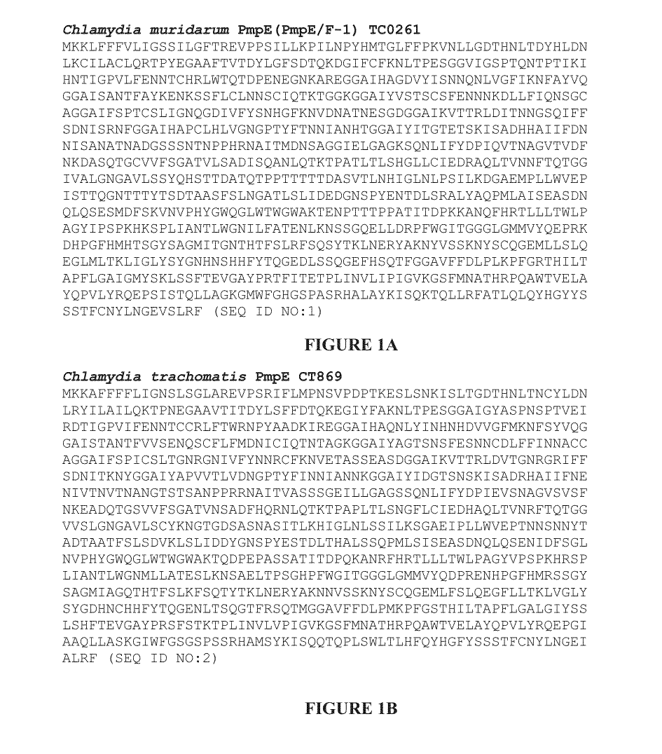

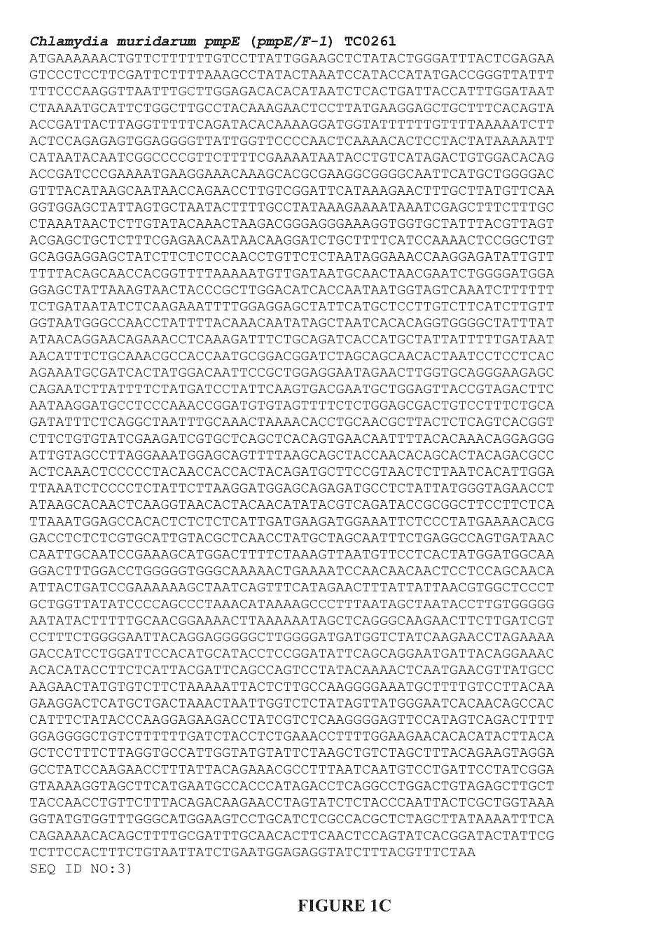

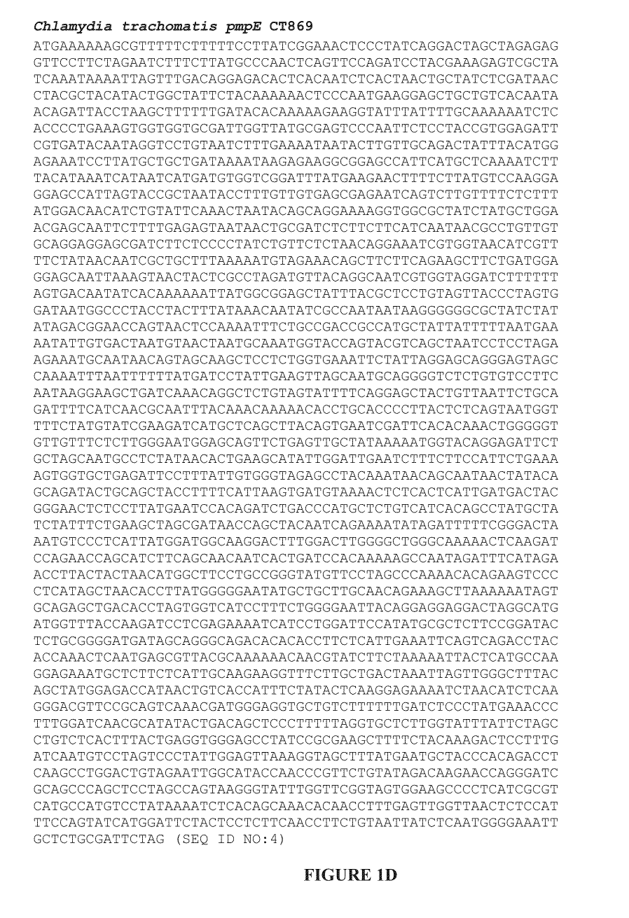

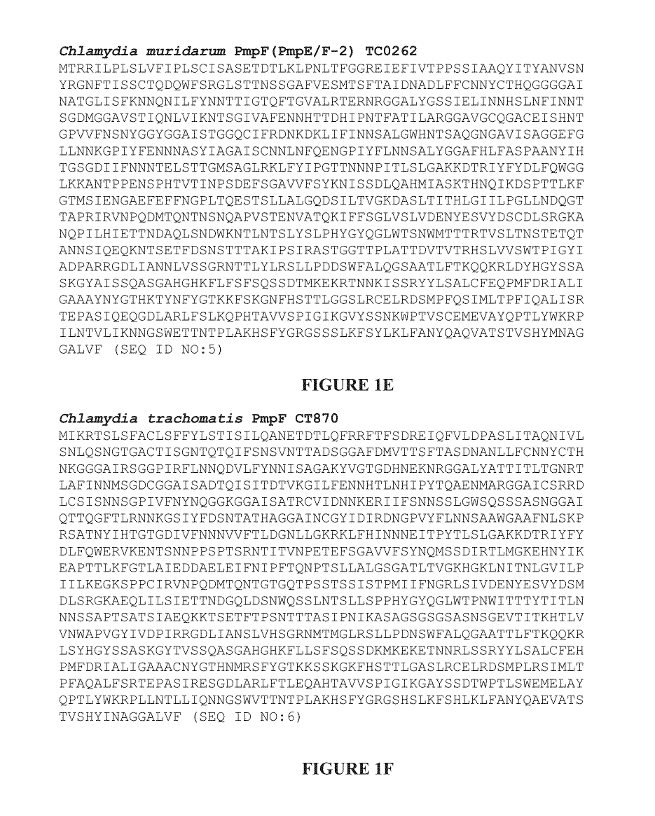

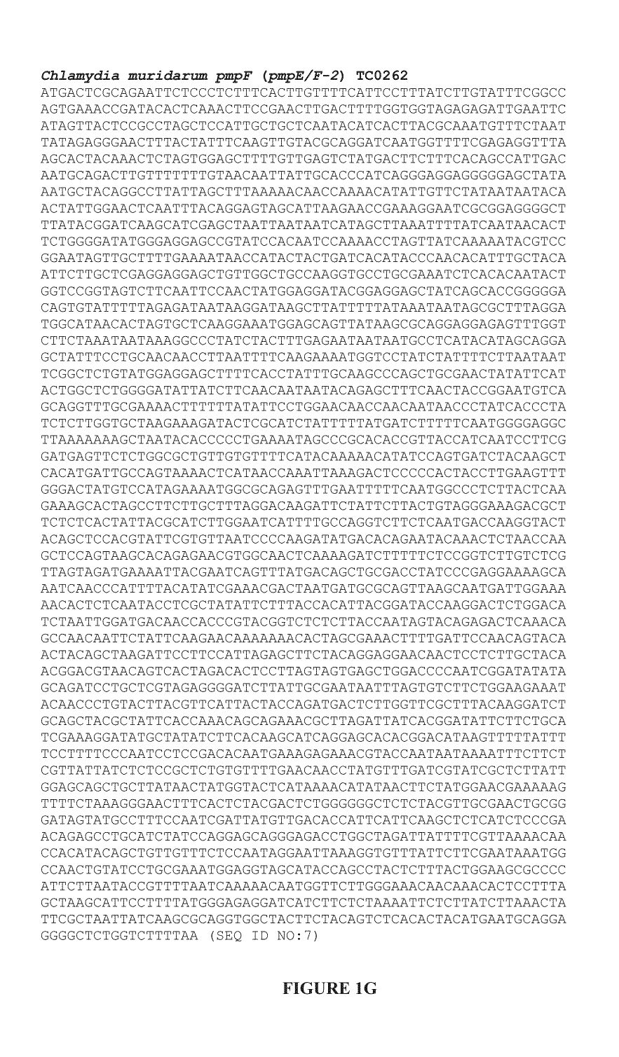

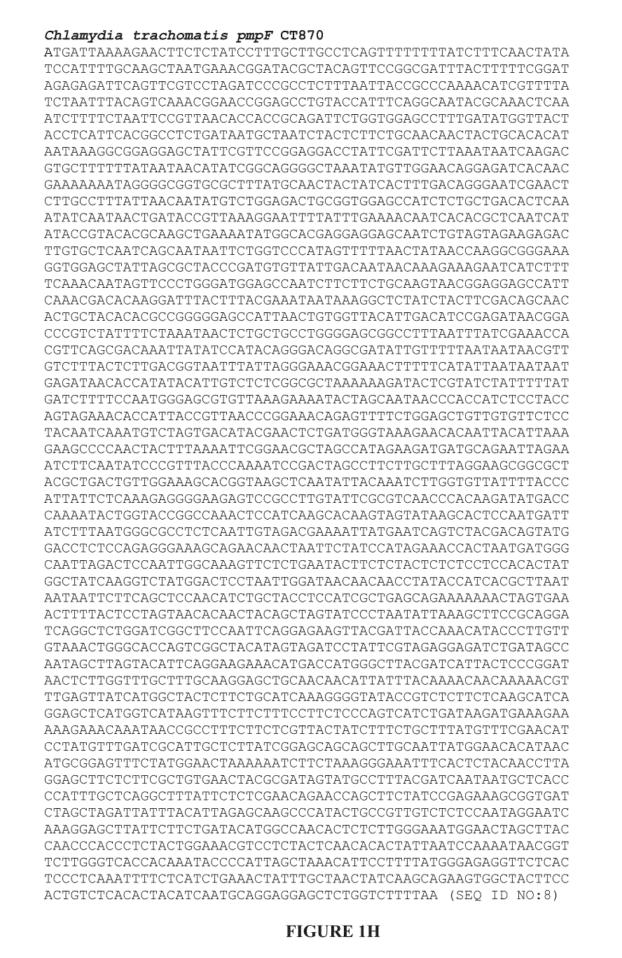

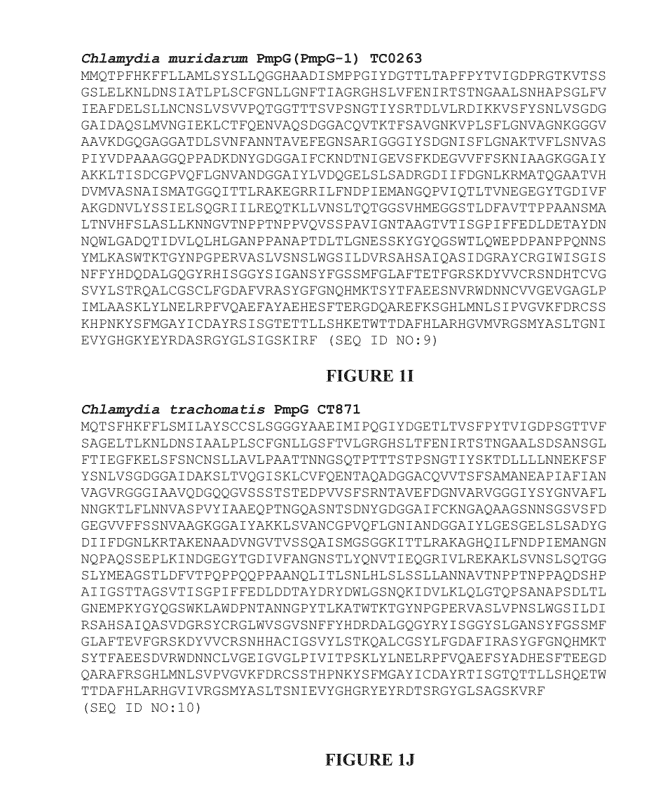

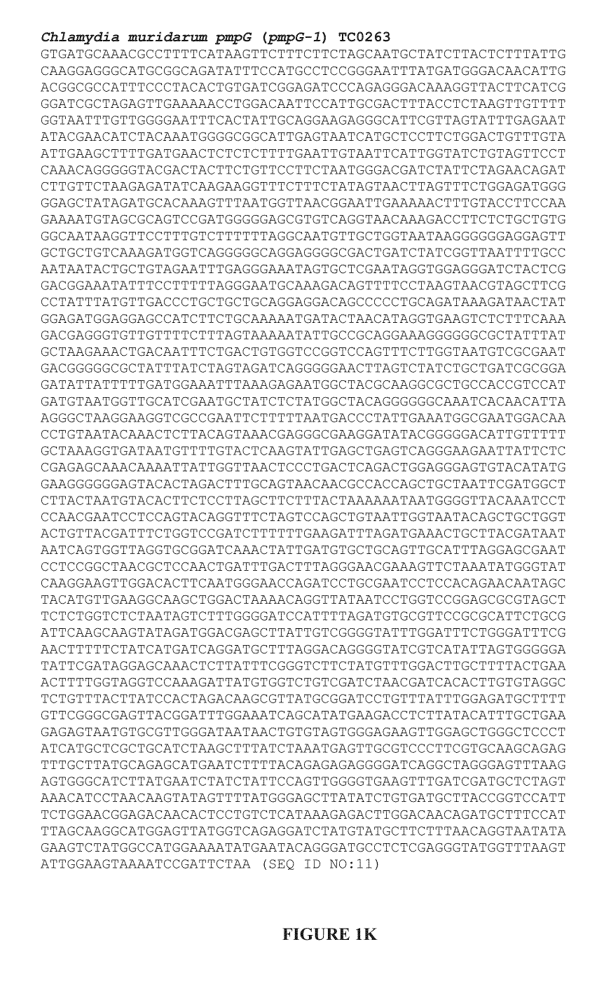

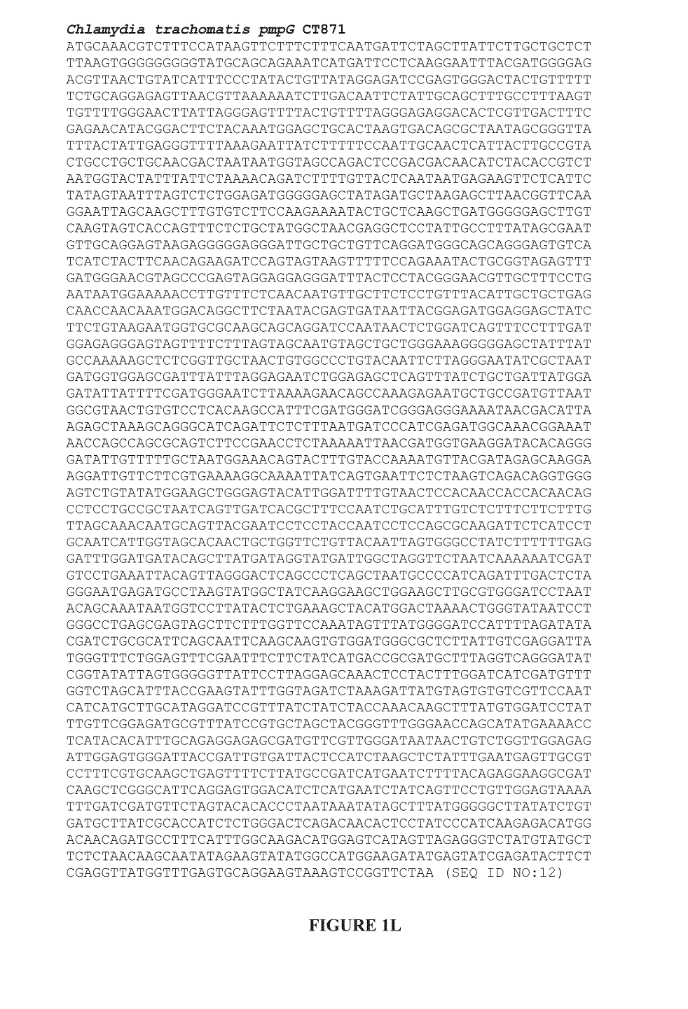

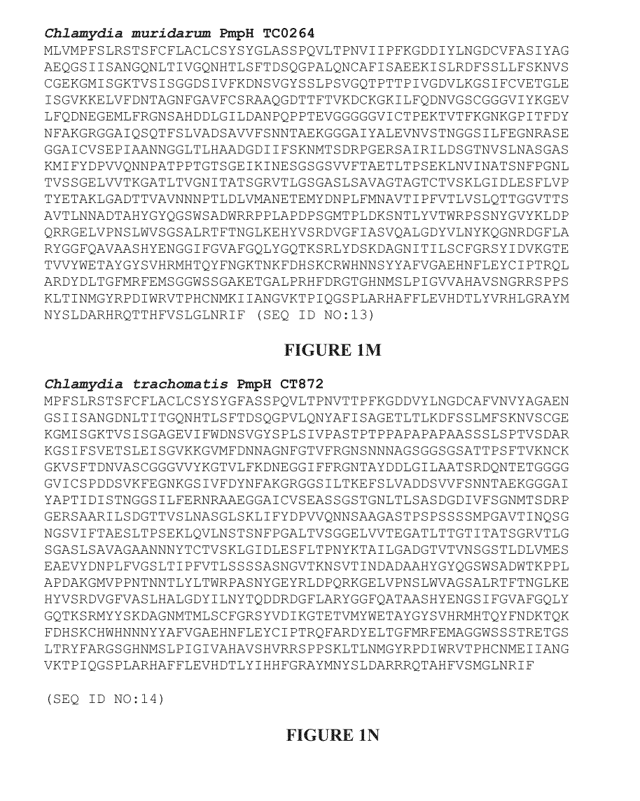

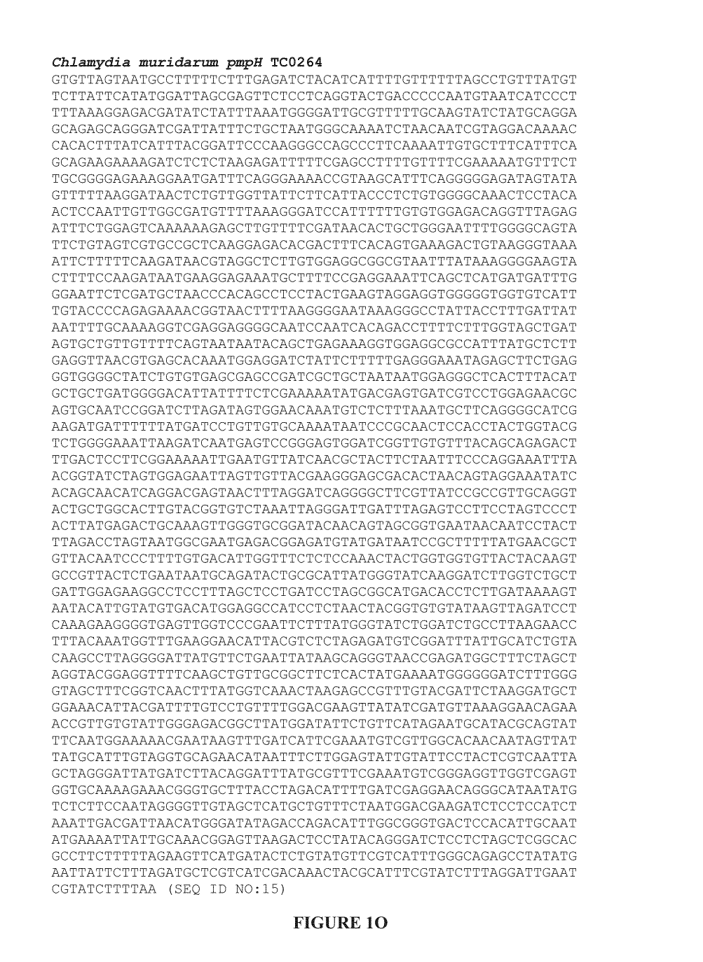

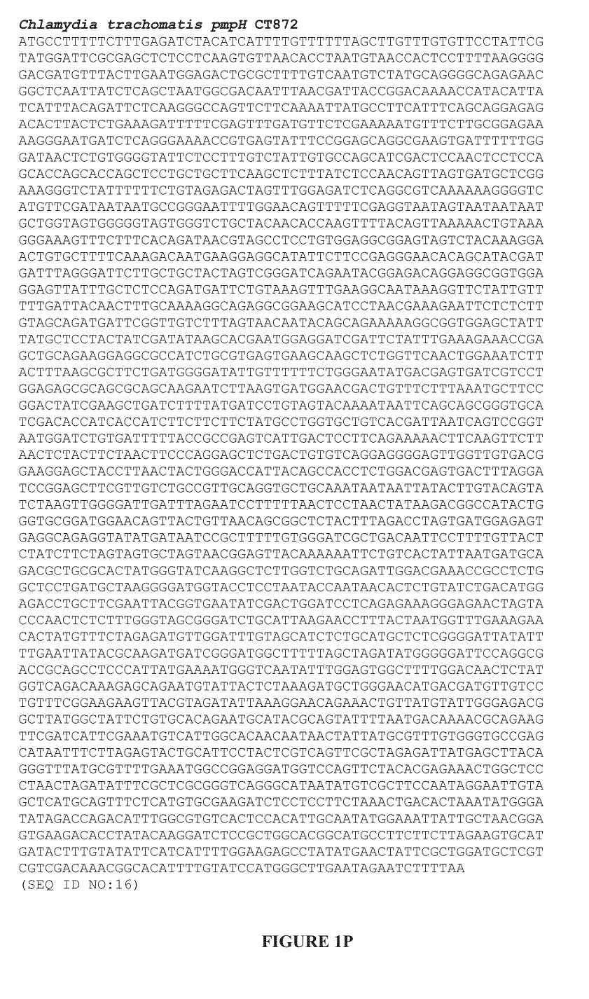

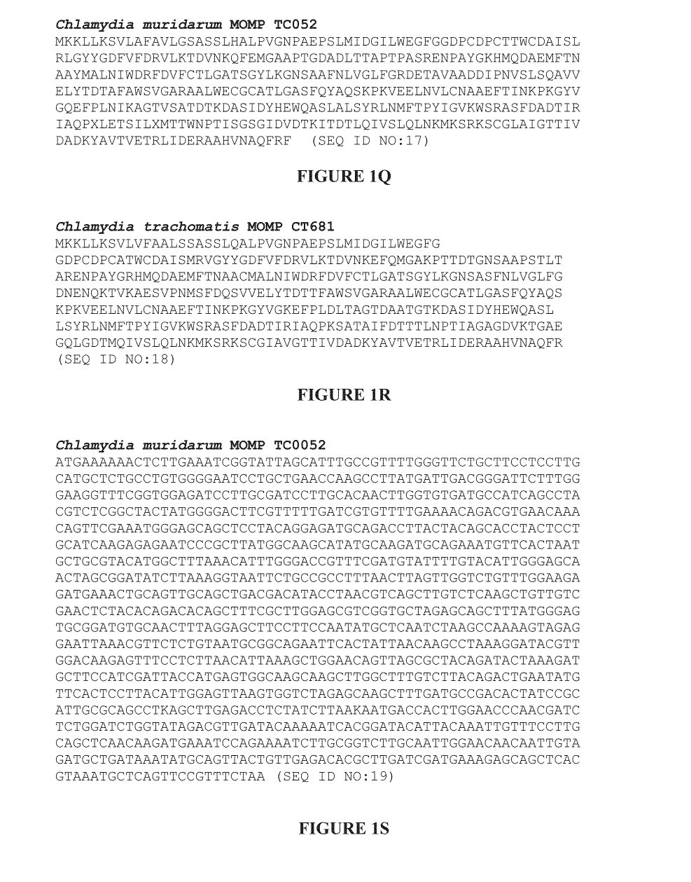

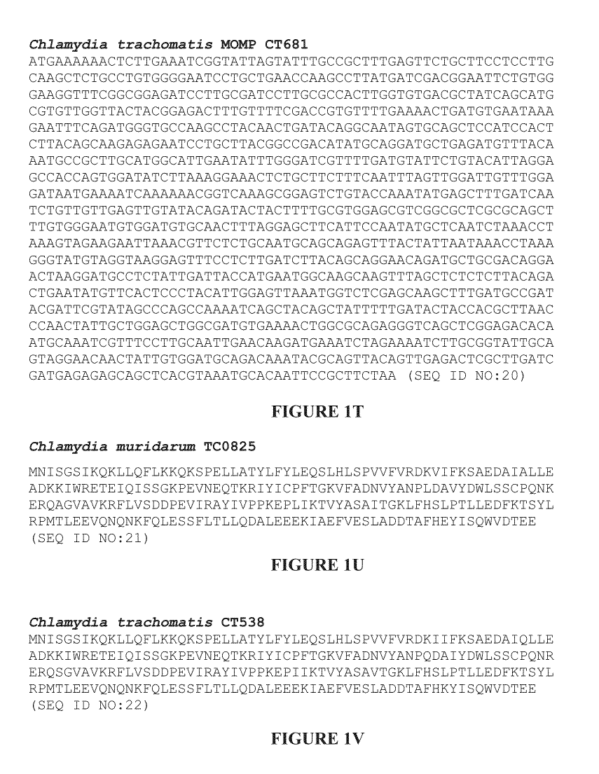

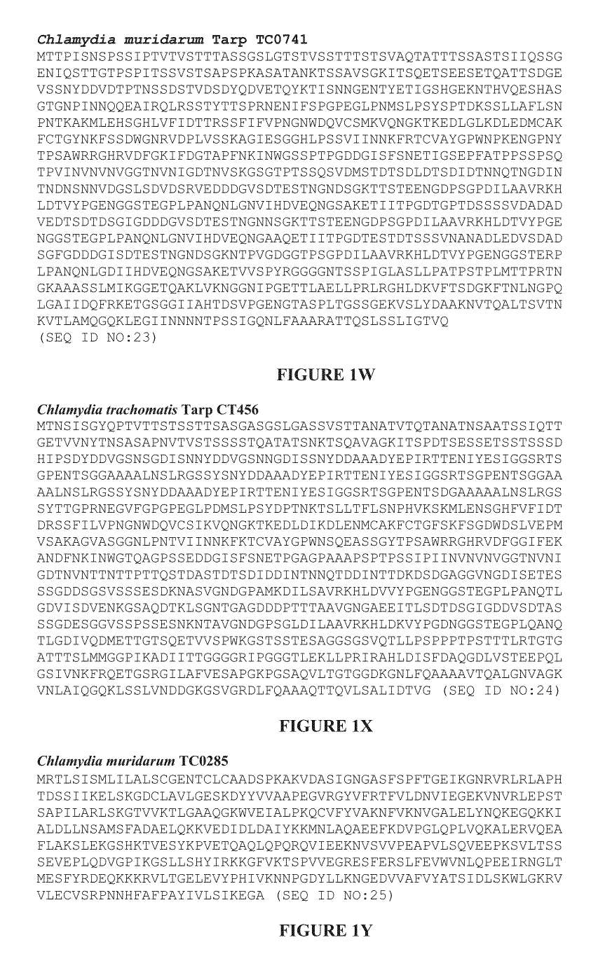

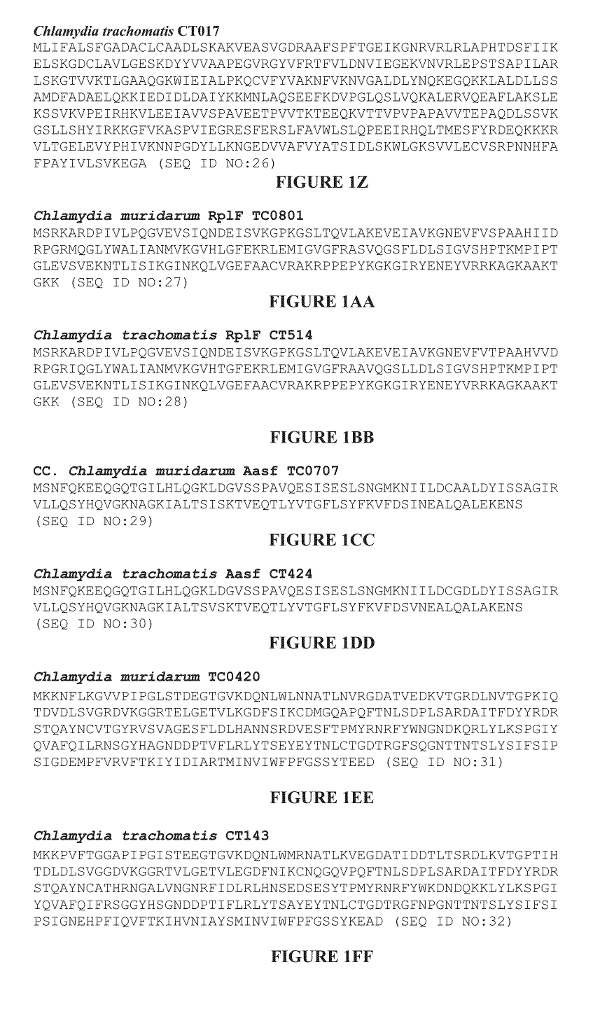

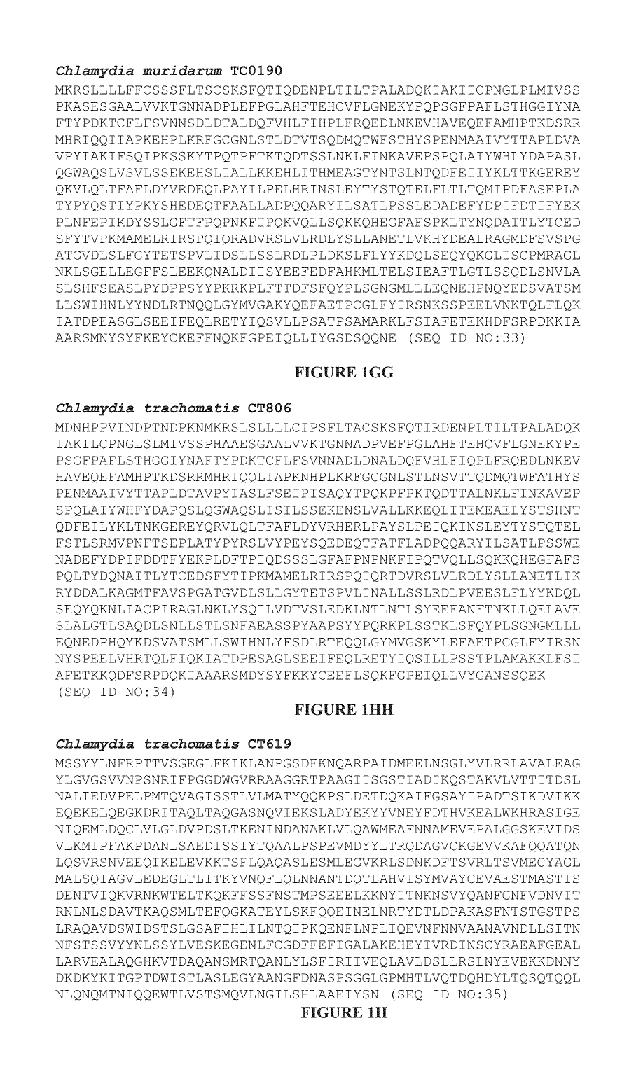

Compounds for use in the compositions and methods according to the disclosure include, without limitation, a fusion protein including the sequence of two or more of the Chlamydia polypeptides described herein, for example, the proteins or polypeptides listed in Tables 1 or 2, or in FIGS. 1A-II, or an immunogenic fragment thereof, as well as a nucleic acid molecule encoding such a fusion protein.

TABLE 1
Homology of C. muridarum-derived source proteins to C. trachomatis,
other bacteria and human

| | | | | C. | | |
|---|---|---|---|---|---|---|
| | | | | trachomatis | Other | Human |
| | Chlamydia | | | Identity | Bacteria | (25% |
| | muridarum | Source | Protein | and | (30% cut | cut |
| Peptide Sequence | Locus # | Proteins | Abbr. | Locus # | off) | off) |
| AFHLFASPAANYIHTG | TC0262 | Polymorphic | PmpF | 61%-CT870 | N | N |
| (SEQ ID NO: 42) | | membrane | | | | |
| | | protein F | | | | |
| NAKTVFLSNVASPIYVDPA | TC0263 | Polymorphic | PmpG | 71%-CT871 | N | N |
| (SEQ ID NO: 43) | | membrane | | | | |
| ASPIYVDPAAAGGQPPA | | protein G | | | | |
| (SEQ ID NO: 44) | | | | | | |
| VKGNEVFVSPAAHIIDRPG | TC0801 | Ribosomal | RplF | 96%-CT514 | Y | N |
| (SEQ ID NO: 45) | | protein L6 | | | | |
| SPGQTNYAAAKAGIIGFS | TC0508 | 3-oxoacyl-(acyl | FabG | 90%-CT237 | Y | Y |
| (SEQ ID NO: 46) | | carrier protein) | | | | (44%) |
| | | reductase | | | | |
| KLDGVSSPAVQESISE | TC0707 | Anti-anti-sigma | Aasf | 96%-CT424 | Y | N |
| (SEQ ID NO: 47) | | factor | | | | |
| IGQEITEPLANTVIA | TC0079 | ATP dependent | ClpP | 92%-CT706 | Y | Y |
| (SEQ ID NO: 48) | | Clp protease, | | | | (56%) |
| | | proteolytic | | | | |
| | | subunit | | | | |
| MTTVHAATATQSVVD | TC0792 | Glyceraldehyde | Gap | 95%-CT505 | Y | Y |
| (SEQ ID NO: 49) | | 3-phosphate | | | | (56%) |
| | | dehydrogenase | | | | |
| DLNVTGPKIQTDVD | TC0420 | Hypothetical | | 75%-CT143 | N | N |
| (SEQ ID NO: 50) | | protein | | | | |
| EGTKIPIGTPIAVFSTEQN | TC0518 | Pyruvate | PdhC | 87%-CT247 | Y | Y |
| (SEQ ID NO: 51) | | dehydrogenase | | | | (38%) |
| SVPSYVYYPSGNRAPVV | TC0884 | Thiol disulfide | DsbD | 73%-CT595 | Y | N |
| (SEQ ID NO: 52) | | interchange | | | | |
| | | protein | | | | |
| YDHIIVTPGANADIL | TC0654 | Oxidoreductase, | | 85%-CT375 | Y | N |
| (SEQ ID NO: 53) | | DadA family | | | | |
| LPLMIVSSPKASESGAA | TC0190 | Metalloprotease, | | 80%-CT806 | N | N |
| (SEQ ID NO: 54) | | insulinase | | | | |
| | | family | | | | |
| GANAIPVHCPIGAESQ | TC0721 | Translation | FusA | 97%-CT437 | Y | Y |
| (SEQ ID NO: 55) | | elongation factor | | | | (43%) |
| VFWLGSKINIIDTPG | | G | | | | |
| (SEQ ID NO: 56) | | | | | | |
| ISRALYTPVNSNQSVG | TC0050 | Translation | Tsf | 89%-CT679 | Y | Y |
| (SEQ ID NO: 57) | | elongation factor | | | | (31%) |
| | | Ts | | | | |
| FEVQLISPVALEEGMR | TC0596 | Translation | Tuf | 95%-CT322 | Y | Y |
| (SEQ ID NO: 58) | | elongation factor | | | | (55%) |
| GDAAYIEKVRELMQ | | Tu | | | | |
| (SEQ ID NO: 59) | | | | | | |
| SRALYAQPMLAISEA | TC0261 | Polymorphic | PmpE | 69%-CT869 | N | N |
| (SEQ ID NO: 60) | | membrane | | | | |
| | | protein E | | | | |
| KPAEEEAGSIVHNAREQ | TC0584 | V-type, ATP | AtpE | 91%-CT310 | N | N |
| (SEQ ID NO: 61) | | synthase | | | | |
| | | subunit E | | | | |
| SPQVLTPNVIIPFKGDD | TC0264 | Polymorphic | PmpH | 76%-CT872 | N | N |
| (SEQ ID NO: 62) | | membrane | | | | |
| | | protein H | | | | |
| SMLIIPALGG | TC0895 | Nucleoside | YggV | 81%-CT606 | Y | Y |
| (SEQ ID NO: 63) | | triphosphatase | | | | (33%) |
| LAAAVMHADSGAILKEK | TC0839 | D-analyl-D- | DacC | 76%-CT551 | Y | N |
| (SEQ ID NO: 64) | | alanine | | | | |
| | | carboxypeptidase | | | | |
| DDPEVIRAYIVPPKEP | TC0825 | Hypothetical | | 91%-CT538 | N | N |
| (SEQ ID NO: 65) | | protein | | | | |
| KIFSPAGLLSAFAKNGA | TC0755 | DNA repair | RecO | 85%-CT470 | N | N |
| (SEQ ID NO: 66) | | protein | | | | |
| DPVDMFQMTKIVSKH | TC0745 | SWIB (YM74) | | 86%-CT460 | Y | Y |
| (SEQ ID NO: 67) | | complex protein | | | | (33%) |
| KLEGIINNNNTPS | TC0741 | Translocated | Tarp | 45%-CT456 | N | N |
| (SEQ ID NO: 68) | | actin-recruiting | | | | |
| | | phosphoprotein | | | | |
| AVPRTSLIF | TC0021 | Exodeoxyribo- | RecD_2 | 81%-CT652 | Y | Y |
| (SEQ ID NO: 69) | | nuclease V, | | | | |
| | | alpha subunit | | | | |
| GGAEVILSRSHPEFVKQ | TC0372 | N utilization | NusA | 97%-CT097 | Y | Y |
| (SEQ ID NO: 70) | | substance | | | | |
| | | protein A | | | | |
| APILARLS | TC0285 | Hypothetical | | 82%-CT017 | N | N |
| (SEQ ID NO: 71) | | protein | | | | |

TABLE 2
Chlamydia trachomatis Source Proteins

| Chlamydia | | |
|---|---|---|
| trachomatis | | Protein |
| Locus# | Source Proteins | Abbreviation |
| CT559 | Yop proteins translocation lipoprotein | CdsJ |
| CT837 | Hypothetical protein CT837 | |
| CT110 | Chaperonin GroEL1 | GroEL1 |
| CT144 | Hypothetical protein CT144 | |
| CT289 | Hypothetical protein CT289 | |
| CT619 | Hypothetical protein CT619 | |
| CT561 | Type III secretion translocase | CdsL |
| CT681 | Major Outer Membrane Protein | MOMP |
| CT664 | FHA domain; homology to adenylate cyclase | |
| CT113 | Clp Protease ATPase | ClpB |
| CT759 | Muramidase (invasin repeat family) | NlpD |
| CT045 | Leucyl aminopeptidase | PepA |
| CT420 | 50S ribosomal protein L21 | Rl21 |
| CT622 | CHLPN 76 kDa Homolog | |
| CT472 | Hypothetical protein CT472 | |
| CT842 | Polyribonucleotide Nucleotidyltransferase | Pnp |
| CT778 | Primosome assembly protein | PriA |

In some embodiments, a compound for use in the compositions and methods according to the disclosure includes, without limitation, a C. muridarum- or C. trachomatis-derived amino acid sequence, such as a fusion protein including an amino acid sequence substantially identical to the sequence of two or more of the polypeptides described herein, for example, those listed in Tables 1 or 2, or in FIGS. 1A-II, or an immunogenic fragment thereof.

In some embodiments, a compound for use in the compositions and methods according to the disclosure includes, without limitation, a C. muridarum- or C. trachomatis-derived nucleic acid molecule, such as a nucleic acid sequence that encodes a fusion protein including an amino acid sequence substantially identical to the sequence of two or more of the polypeptides described herein, for example, those listed in Tables 1 or 2, or in Figures FIGS. 1A-II, or an immunogenic fragment thereof.

In some embodiments, a compound for use in the compositions and methods according to the disclosure includes, without limitation, a C. muridarum- or C. trachomatis-derived nucleic acid molecule, such as a nucleic acid sequence substantially identical to the nucleic acid sequence of two or more of the polypeptides described herein, for example, those listed in Tables 1 or 2, or in FIGS. 1A-II, or an immunogenic fragment thereof.

In alternative embodiments, a compound for use in the compositions and methods according to the disclosure includes, without limitation, a fusion protein including two or more of a Chlamydia polypeptide, such as Polymorphic membrane protein G (PmpG), Polymorphic membrane protein F (PmpF), Polymorphic membrane protein E (PmpE), Polymorphic membrane protein H (PmpH), Ribosomal protein L6 (RplF), Anti-anti-sigma factor (Aasf), Translocated actin-recruiting phosphoprotein (Tarp), hypothetical protein corresponding to locus tag CT143/TC0420, metalloprotease, insulinase family (CT806/TC0190), hypothetical protein corresponding to locus tag CT538/TC0825, hypothetical protein corresponding to locus tag CT017/TC0285, hypothetical protein corresponding to locus tag CT619, or MOMP, or an immunogenic fragment thereof.

In alternative embodiments, a compound for use in the compositions and methods according to the disclosure includes, without limitation, a fusion protein including two or more of a Chlamydia-derived amino acid sequence, such as a fusion protein including an amino acid sequence substantially identical to the sequence of two or more of the following polypeptides: Polymorphic membrane protein G (PmpG), Polymorphic membrane protein F (PmpF), Polymorphic membrane protein E (PmpE), Polymorphic membrane protein H (PmpH), Ribosomal protein L6 (RplF), Anti-anti-sigma factor (Aasf), Translocated actin-recruiting phosphoprotein (Tarp), hypothetical protein corresponding to locus tag CT143/TC0420, metalloprotease, insulinase family (CT806/TC0190), hypothetical protein corresponding to locus tag CT538/TC0825, hypothetical protein corresponding to locus tag CT017/TC0285, hypothetical protein corresponding to locus tag CT619, or MOMP, or an immunogenic fragment thereof.

In alternative embodiments, a compound for use in the compositions and methods according to the disclosure includes, without limitation, a fusion protein encoded by two or more of a Chlamydia-derived nucleic acid sequence, such as a nucleic acid sequence that encodes a fusion protein including an amino acid sequence substantially identical to the sequence of two or more of the following polypeptides: Polymorphic membrane protein G (PmpG), Polymorphic membrane protein F (PmpF), Polymorphic membrane protein E (PmpE), Polymorphic membrane protein H (PmpH), Ribosomal protein L6 (RplF), Anti-anti-sigma factor (Aasf), Translocated actin-recruiting phosphoprotein (Tarp), hypothetical protein corresponding to locus tag CT143/TC0420, metalloprotease, insulinase family (CT806/TC0190), hypothetical protein corresponding to locus tag CT538/TC0825, hypothetical protein corresponding to locus tag CT017/TC0285, hypothetical protein corresponding to locus tag CT619, or MOMP, or an immunogenic fragment thereof.

In alternative embodiments, a compound for use in the compositions and methods according to the disclosure includes, without limitation, a fusion protein encoded by two or more of a Chlamydia-derived nucleic acid sequence, such as a nucleic acid sequence substantially identical to the nucleic acid sequence encoding two or more of the following polypeptides: Polymorphic membrane protein G (PmpG), Polymorphic membrane protein F (PmpF), Polymorphic membrane protein E (PmpE), Polymorphic membrane protein H (PmpH), Ribosomal protein L6 (RplF), Anti-anti-sigma factor (Aasf), Translocated actin-recruiting phosphoprotein (Tarp), hypothetical protein corresponding to locus tag CT143/TC0420, metalloprotease, insulinase family (CT806/TC0190), hypothetical protein corresponding to locus tag CT538/TC0825, hypothetical protein corresponding to locus tag CT017/TC0285, hypothetical protein corresponding to locus tag CT619, or MOMP, or an immunogenic fragment thereof.

In some embodiments, a compound for use in the compositions and methods according to the disclosure includes, without limitation, a fusion protein including two or more of PmpG, PmpF, PmpE, PmpH, RplF, Aasf, Tarp, TC0420, TC0190, TC0825, TC0285, CT619, MOMP, or an immunogenic fragment thereof, or a nucleic acid molecule encoding such a fusion protein.

In some embodiments, a compound for use in the compositions and methods according to the disclosure includes, without limitation, a fusion protein including three or more of PmpG, PmpF, PmpE, PmpH, RplF, Aasf, Tarp, TC0420, TC0190, TC0825, TC0285, CT619, MOMP, or an immunogenic fragment thereof, or a nucleic acid molecule encoding such a fusion protein.

In some embodiments, a compound for use in the compositions and methods according to the disclosure includes, without limitation, a fusion protein including four or more of PmpG, PmpF, PmpE, PmpH, RplF, Aasf, Tarp, TC0420, TC0190, TC0825, TC0285, CT619, MOMP, or an immunogenic fragment thereof, or a nucleic acid molecule encoding such a fusion protein.

In some embodiments, a compound for use in the compositions and methods according to the disclosure includes, without limitation, a fusion protein including two or more of the following Chlamydia proteins/antigens: PmpG, PmpE, PmpF, PmpH and, optionally, MOMP, or an immunogenic fragment thereof, or a nucleic acid molecule encoding such a fusion protein.

In some embodiments, a compound for use in the compositions and methods according to the disclosure includes, without limitation, a fusion protein including two or more of the following Chlamydia proteins/antigens: PmpG, PmpE, PmpF and TC0420 and, optionally, MOMP, or an immunogenic fragment thereof, or a nucleic acid molecule encoding such a fusion protein.

In some embodiments, a compound for use in the compositions and methods according to the disclosure includes, without limitation, a fusion protein including the following Chlamydia proteins/antigens: PmpG and MOMP, or an immunogenic fragment thereof, or a nucleic acid molecule encoding such a fusion protein. In alternative embodiments a fusion protein including only the following Chlamydia proteins/antigens: PmpG and MOMP, or an immunogenic fragment thereof, or a nucleic acid molecule encoding such a fusion protein.

In some embodiments, a compound for use in the compositions and methods according to the disclosure includes, without limitation, a fusion protein including the following Chlamydia proteins/antigens: PmpG and PmpF, or an immunogenic fragment thereof, or a nucleic acid molecule encoding such a fusion protein. In alternative embodiments, a compound for use in the compositions and methods according to the disclosure includes a fusion protein including only the following Chlamydia proteins/antigens: PmpG and PmpF, or an immunogenic fragment thereof, or a nucleic acid molecule encoding such a fusion protein.

In some embodiments, a compound for use in the compositions and methods according to the disclosure includes, without limitation, a fusion protein including the following Chlamydia proteins/antigens: PmpG and PmpH, or an immunogenic fragment thereof, or a nucleic acid molecule encoding such a fusion protein. In alternative embodiments, a compound for use in the compositions and methods according to the disclosure includes a fusion protein including only the following Chlamydia proteins/antigens: PmpG and PmpH, or an immunogenic fragment thereof, or a nucleic acid molecule encoding such a fusion protein.

In some embodiments, a compound for use in the compositions and methods according to the disclosure includes, without limitation, a fusion protein including the following Chlamydia proteins/antigens: PmpE and PmpF, or an immunogenic fragment thereof, or a nucleic acid molecule encoding such a fusion protein. In alternative embodiments, a compound for use in the compositions and methods according to the disclosure includes a fusion protein including only the following Chlamydia proteins/antigens: PmpE and PmpF, or an immunogenic fragment thereof, or a nucleic acid molecule encoding such a fusion protein.

In some embodiments, a compound for use in the compositions and methods according to the disclosure includes, without limitation, a fusion protein including the following Chlamydia proteins/antigens: PmpG and TC0420, or an immunogenic fragment thereof, or a nucleic acid molecule encoding such a fusion protein. In alternative embodiments, a compound for use in the compositions and methods according to the disclosure includes a fusion protein including only the following Chlamydia proteins/antigens: PmpG and TC0420, or an immunogenic fragment thereof, or a nucleic acid molecule encoding such a fusion protein.

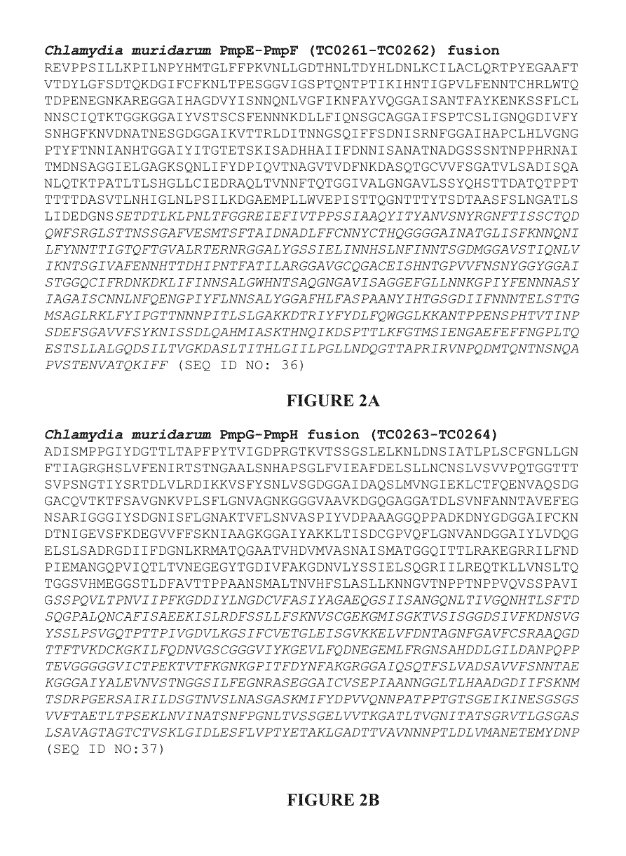

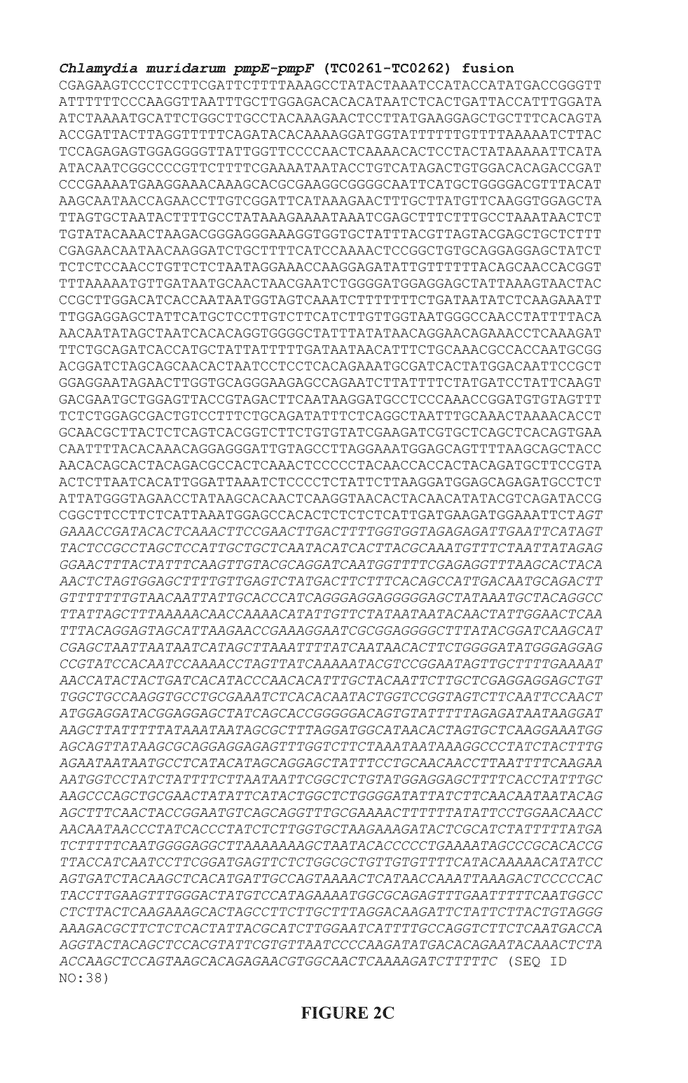

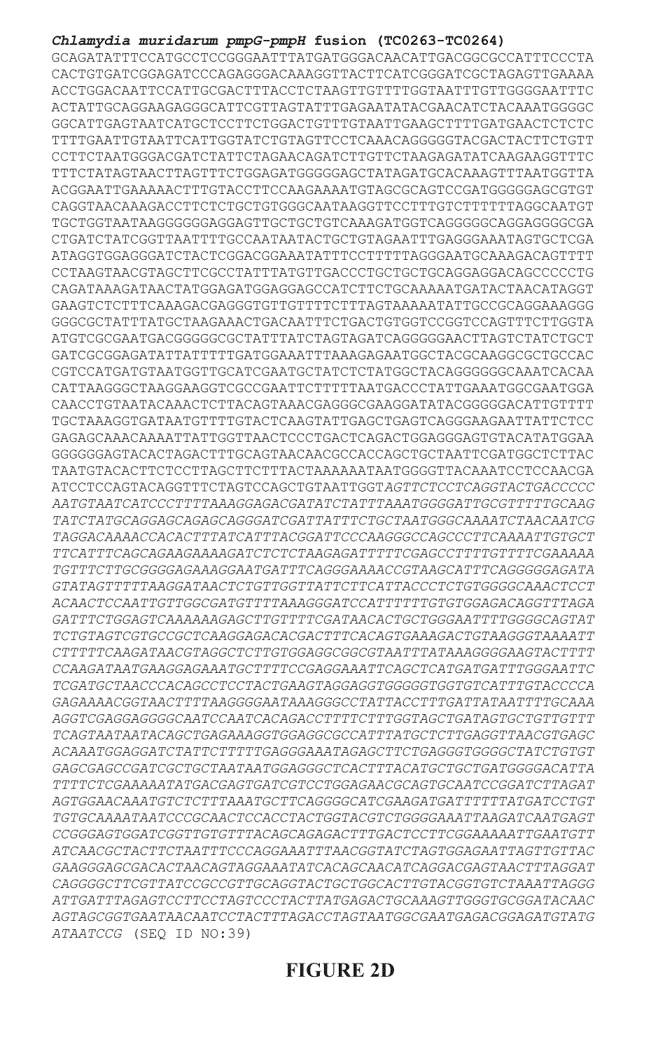

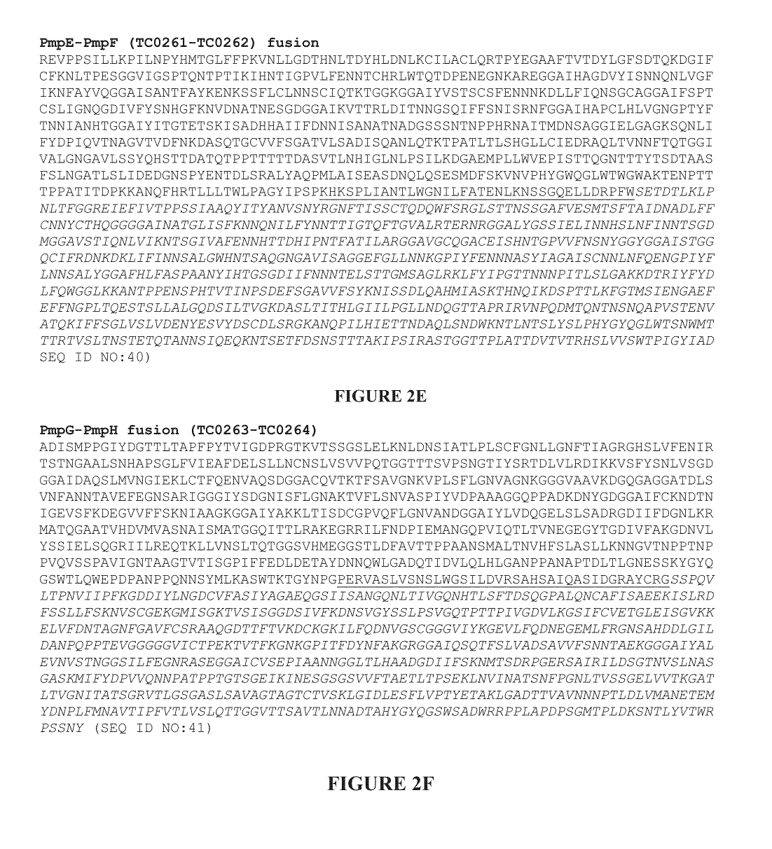

In alternative embodiments, a compound for use in the compositions and methods according to the disclosure includes, without limitation, one or more of the fusion proteins described in FIGS. 2A-F.

It is to be understood that compositions according to the disclosure can include mixtures of fusion proteins and individual (non-fusion) proteins, or immunogenic fragments thereof, as long as at least one polypeptide in the mixture is a fusion protein.

In some embodiments, a composition according to the disclosure includes, without limitation, a mixture of two or more fusion proteins, and optionally individual antigens, such as a mixture of PmpG/PmpH and PmpE/PmpF and optionally, MOMP; and/or PmpG/TC0420 and PmpE/PmpF and optionally, MOMP, or an immunogenic fragment thereof.

In alternative embodiments, compositions according to the disclosure further include, without limitation, mixtures of fusion proteins, where MOMP, or an immunogenic fragment thereof, is part of a fusion protein.

In alternative embodiments, compounds for use in the compositions and methods according to the disclosure include, without limitation, a fusion protein including two or more of a C. trachomatis polypeptide, such as Ribosomal protein L6 (RpIF, gi:3328951), Anti anti sigma factor (Aasf, gi: 15605151), Polymorphic membrane protein G (PmpG, gi:3329346), Hypothetical protein (TC0420, gi: 15604862), Polymorphic membrane protein F (PmpF, gi:3329345), or major outer membrane protein 1 (MOMP) (gi:3329133), or an immunogenic fragment or portion thereof. Examples of fragments or portions of such C. trachomatis polypeptides include, without limitation, amino acids 25-512 of PmpG (PmpG₂₅₋₅₁₂), amino acids 26-585 of PmpF (PmpF₂₆₋₅₈₅), or amino acids 22-393 of MOMP.

In alternative embodiments, compounds for use in the compositions and methods according to the disclosure further include, without limitation, a fusion protein including two or more of a C. muridarum polypeptide, such as Ribosomal protein L6 (RpIF, gi: 15835415), Anti anti sigma factor (Aasf, gi: 15835322), Polymorphic membrane protein G (PmpG or PmpG-1, gi: 15834883), Hypothetical protein TC0420(gi: 15835038), Polymorphic membrane protein F (PmpF or PmpE/F, gi: 15834882), or major outer membrane protein 1 (MOMP, gi7190091), or an immunogenic fragment or portion thereof. Examples of such fragments or portions of C. muridarum polypeptides include, without limitation, amino acids 25-500 of PmpG-1 (PmpG-1₂₅₋₅₀₀), amino acids 25-575 of PmpE/F-2 (PmpE/F-2₂₅₋₅₇₅), or amino acids 23-387 of MOMP.

In alternative embodiments, an immunogenic fragment or portion of a Chlamydia polypeptide includes the region of the polypeptide that is generally exposed on the surface of the polypeptide. In alternative embodiments, such a fragment or portion of a Chlamydia polypeptide includes the passenger domain of a Pmp polypeptide e.g., the domain located between the signal sequence and the translocation unit.

In alternative embodiments, an immunogenic fragment or portion of a C. muridarum polypeptide includes the passenger domain, or a portion thereof, of a C. muridarum Pmp polypeptide, for example, amino acids 18 to 667 of PmpE; amino acids 18 to 575 of PmpE; amino acids 20 to 722 of PmpF; amino acids 20 to 575 of PmpF; amino acids 25 to 675 of PmpG; amino acids 25 to 555 of PmpG; amino acids 27 to 653 of PmpH; or amino acids 27 to 575 of PmpH. In alternative embodiments, an immunogenic fragment or portion of a C. trachomatis polypeptide includes the passenger domain, or a portion thereof, of a C. trachomatis Pmp polypeptide. In alternative embodiments, an immunogenic fragment or portion of a Chlamydia polypeptide includes a peptide sequence as described in Table 1. In alternative embodiments, passenger domain fragments can be about 550 amino acids in length, or about 600 amino acids from the N-terminus of the Pmp polypeptide, or less. In alternative embodiments, an immunogenic fragment can be about 25 to about 600 amino acids in length, for example, about 25, 50, 75, 100, 125, 150, 175, 200, 225, 250, 275, 300, 325, 350, 375, 400, 425, 450, 475, 500, 525, 550, 575, 600, or any integer within these values.

In general, it is to be understood that the sequences of polypeptides and amino acids referenced herein correspond to those indicated in the locus tags referenced in the C. trachomatis genome sequence and/or the C. muridarum genome sequence. It is also to be understood that the nucleic acid sequences corresponding to the locus tags can be obtained from the C. trachomatis genome sequence and/or the C. muridarum genome sequence.

In some embodiments, fusion proteins for use according to the disclosure consist essentially of two Chlamydia polypeptides, or an immunogenic fragment thereof, as described herein.

In some embodiments, fusion proteins for use according to the disclosure consist essentially of three Chlamydia polypeptides, or an immunogenic fragment thereof, as described herein.

In some embodiments, fusion proteins for use according to the disclosure consist essentially of four Chlamydia polypeptides, or an immunogenic fragment thereof, as described herein.

In some embodiments, fusion proteins for use according to the disclosure include at least two Chlamydia polypeptides, or an immunogenic fragment thereof, for example, at least 2, 3, 4, 5, or more.

By “fusion protein” or “chimeric protein” is meant a recombinant protein or polypeptide in which at least two Chlamydia proteins or antigens, as for example, described herein or set forth in Tables 1 or 2, or FIGS. 1A-II, are present in a single, recombinant polypeptide. It is to be understood that the individual Chlamydia proteins or antigens that make up the fusion protein can be present in the fusion protein in any order or orientation. For example, in some embodiments, the individual Chlamydia proteins or antigens can be present in the fusion protein in the opposite direction relative to the naturally occurring (i.e. N-terminal to C-terminal reversed) direction. In some embodiments, the fusion protein can include full-length Chlamydia proteins or antigens. In alternative embodiments, the fusion protein can include portions or fragments of Chlamydia proteins or antigens, such as regions of the Chlamydia proteins or antigens exposed to the surface (“passenger domains”) or including immunodominant epitopes.

In some embodiments, a fusion protein may be provided in combination with a heterologous peptide or polypeptide, such as an epitope tag.

In some embodiments, the individual Chlamydia protein or antigen sequences may be joined directly to each other.

In some embodiments, a fusion protein may be provided in combination with a heterologous peptide or polypeptide, such as a linker or spacer that, for example, enables correct folding and/or presentation and/or expression of the fusion protein. The linker or spacer may be placed between each individual Chlamydia protein or antigen sequence, or may be placed between only some of the individual Chlamydia protein or antigen sequences present in the fusion protein.

In alternative embodiments, the linker may be a heterologous linker, such as a sequence (e.g., an alpha helical sequence) from another Chlamydia protein or antigen or from a non-adjacent location of the Chlamydia proteins or antigens forming the fusion protein, or may be a homologous linker, such as a sequence (e.g., an alpha helical sequence) from one of the Chlamydia proteins or antigens forming the fusion protein and adjacent to the sequence used in the fusion protein. For example, the passenger domains of the Pmp fusion partners can be connected via an alpha-helical linker (shown in underline in FIGS. 2E-F) polypeptide. The linker polypeptides can be derived from polypeptide sequences of one of the fusion protein partners. For example, the linker for the PmpE-PmpF fusion protein includes a sequence from PmpE and the linker for the PmpG-PmpH fusion protein includes a sequence from PmpG (FIGS. 2E-F).

It is well known in the art that some modifications and changes can be made in the structure of a polypeptide without substantially altering the biological function of that polypeptide e.g., its ability to be cleaved into smaller peptides that are capable of binding to MHC proteins, to obtain a biologically equivalent polypeptide. Accordingly, it will be appreciated by a person of skill in the art that the numerical designations of the positions of amino acids within a sequence are relative to the specific sequence. Also the same positions may be assigned different numerical designations depending on the way in which the sequence is numbered and the sequence chosen. Furthermore, sequence variations such as insertions or deletions, may change the relative position and subsequently the numerical designations of particular amino acids at and around a site.

A “protein,” “peptide,” or “polypeptide” is any chain of two or more amino acids, including naturally occurring or non-naturally occurring amino acids or amino acid analogues, regardless of post-translational modification (e.g., glycosylation or phosphorylation). An “amino acid sequence,” “polypeptide,” “peptide,” or “protein” of the invention may include peptides or proteins that have abnormal linkages, cross links and end caps, non-peptidyl bonds or alternative modifying groups. Such modified peptides are also within the scope of the invention. The term “modifying group” is intended to include structures that are directly attached to the peptidic structure (e.g., by covalent coupling), as well as those that are indirectly attached to the peptidic structure (e.g., by a stable non-covalent association or by covalent coupling to additional amino acid residues, or mimetics, analogues or derivatives thereof, which may flank the core peptidic structure). For example, the modifying group can be coupled to the amino-terminus or carboxy-terminus of a peptidic structure, or to a peptidic or peptidomimetic region flanking the core domain.

Alternatively, the modifying group can be coupled to a side chain of at least one amino acid residue of a peptidic structure, or to a peptidic or peptido-mimetic region flanking the core domain (e.g., through the epsilon amino group of a lysyl residue(s), through the carboxyl group of an aspartic acid residue(s) or a glutamic acid residue(s), through a hydroxy group of a tyrosyl residue(s), a serine residue(s) or a threonine residue(s) or other suitable reactive group on an amino acid side chain). Modifying groups covalently coupled to the peptidic structure can be attached by means and using methods well known in the art for linking chemical structures, including, for example, amide, alkylamino, carbamate or urea bonds.

In one aspect of the invention, polypeptides of the present invention also extend to biologically equivalent peptides or “variants” that differ from a portion of the sequence of the polypeptides of the present invention by conservative amino acid substitutions, or differ by non-conservative substitutions that do not affect biological function e.g., immunogenicity. As used herein, the term “conserved amino acid substitutions” refers to the substitution of one amino acid for another at a given location in the peptide, where the substitution can be made without substantial loss of the relevant function. In making such changes, substitutions of like amino acid residues can be made on the basis of relative similarity of side-chain substituents, for example, their size, charge, hydrophobicity, hydrophilicity, and the like, and such substitutions may be assayed for their effect on the function of the peptide by routine testing.

As used herein, the term “amino acids” means those L-amino acids commonly found in naturally occurring proteins, D-amino acids and such amino acids when they have been modified. Accordingly, amino acids of the invention may include, for example: 2-Aminoadipic acid; 3-Aminoadipic acid; beta-Alanine; beta-Aminopropionic acid; 2-Aminobutyric acid; 4-Aminobutyric acid; piperidinic acid; 6-Aminocaproic acid; 2-Aminoheptanoic acid; 2-Aminoisobutyric acid; 3-Aminoisobutyric acid; 2-Aminopimelic acid; 2,4 Diaminobutyric acid; Desmosine; 2,2′-Diaminopimelic acid; 2,3-Diaminopropionic acid; N-Ethylglycine; N-Ethylasparagine; Hydroxylysine; allo-Hydroxylysine; 3-Hydroxyproline; 4-Hydroxyproline; Isodesmosine; allo-Isoleucine; N-Methylglycine; sarcosine; N-Methylisoleucine; 6-N-methyllysine; N-Methylvaline; Norvaline; Norleucine; and Ornithine.

In some embodiments, conserved amino acid substitutions may be made where an amino acid residue is substituted for another having a similar hydrophilicity value (e.g., within a value of plus or minus 2.0, or plus or minus 1.5, or plus or minus 1.0, or plus or minus 0.5), where the following may be an amino acid having a hydropathic index of about −1.6 such as Tyr (−1.3) or Pro (−1.6) are assigned to amino acid residues (as detailed in U.S. Pat. No. 4,554,101, incorporated herein by reference): Arg (+3.0); Lys (+3.0); Asp (+3.0); Glu (+3.0); Ser (+0.3); Asn (+0.2); Gin (+0.2); Gly (0); Pro (−0.5); Thr (−0.4); Ala (−0.5); His (−0.5); Cys (−1.0); Met (−1.3); Val (−1.5); Leu (−1.8); lie (−1.8); Tyr (−2.3); Phe (−2.5); and Trp (−3.4).

In alternative embodiments, conservative amino acid substitutions may be made where an amino acid residue is substituted for another having a similar hydropathic index (e.g., within a value of plus or minus 2.0, or plus or minus 1.5, or plus or minus 1.0, or plus or minus 0.5). In such embodiments, each amino acid residue may be assigned a hydropathic index on the basis of its hydrophobicity and charge characteristics, as follows: He (+4.5); Val (+4.2); Leu (+3.8); Phe (+2.8); Cys (+2.5); Met (+1.9); Ala (+1.8); Gly (−0.4); Thr (−0.7); Ser (−0.8); Trp (−0.9); Tyr (−1.3); Pro (−1.6); His (−3.2); Glu (−3.5); Gin (−3.5); Asp (−3.5); Asn (−3.5); Lys (−3.9); and Arg (−4.5).

In alternative embodiments, conservative amino acid substitutions may be made using publicly available families of similarity matrices (60, 70, 102, 103, 94, 104, 86) The PAM matrix is based upon counts derived from an evolutionary model, while the Blosum matrix uses counts derived from highly conserved blocks within an alignment. A similarity score of above zero in either of the PAM or Blosum matrices may be used to make conservative amino acid substitutions.

In alternative embodiments, conservative amino acid substitutions may be made where an amino acid residue is substituted for another in the same class, where the amino acids are divided into non-polar, acidic, basic and neutral classes, as follows: non-polar: Ala, Val, Leu, He, Phe, Trp, Pro, Met; acidic: Asp, Glu; basic: Lys, Arg, His; neutral: Gly, Ser, Thr, Cys, Asn, Gln, Tyr.

Conservative amino acid changes can include the substitution of an L-amino acid by the corresponding D-amino acid, by a conservative D-amino acid, or by a naturally-occurring, non-genetically encoded form of amino acid, as well as a conservative substitution of an L-amino acid. Naturally-occurring non-genetically encoded amino acids include beta-alanine, 3-amino-propionic acid, 2,3-diamino propionic acid, alpha-aminoisobutyric acid, 4-amino-butyric acid, N-methylglycine (sarcosine), hydroxyproline, ornithine, citrulline, t-butylalanine, t-butylglycine, N-methylisoleucine, phenylglycine, cyclohexylalanine, norleucine, norvaline, 2-napthylalanine, pyridylalanine, 3-benzothienyl alanine, 4-chlorophenylalanine, 2-fluorophenylalanine, 3-fluorophenylalanine, 4-fluorophenylalanine, penicillamine, 1,2,3,4-tetrahydro-isoquinoline-3-carboxylix acid, beta-2-thienylalanine, methionine sulfoxide, homoarginine, N-acetyl lysine, 2-amino butyric acid, 2-amino butyric acid, 2,4,-diamino butyric acid, p-aminophenylalanine, N-methylvaline, homocysteine, homoserine, cysteic acid, epsilon-amino hexanoic acid, delta-amino valeric acid, or 2,3-diaminobutyric acid.

In alternative embodiments, conservative amino acid changes include changes based on considerations of hydrophilicity or hydrophobicity, size or volume, or charge. Amino acids can be generally characterized as hydrophobic or hydrophilic, depending primarily on the properties of the amino acid side chain. A hydrophobic amino acid exhibits a hydrophobicity of greater than zero, and a hydrophilic amino acid exhibits a hydrophilicity of less than zero, based on the normalized consensus hydrophobicity scale of Eisenberg et al. (Ann. Rev. Biochem. 53: 595-623, 1984). Genetically encoded hydrophobic amino acids include Gly, Ala, Phe, Val, Leu, He, Pro, Met and Trp, and genetically encoded hydrophilic amino acids include Thr, His, Glu, Gln, Asp, Arg, Ser, and Lys. Non-genetically encoded hydrophobic amino acids include t-butylalanine, while non-genetically encoded hydrophilic amino acids include citrulline and homocysteine.

Hydrophobic or hydrophilic amino acids can be further subdivided based on the characteristics of their side chains. For example, an aromatic amino acid is a hydrophobic amino acid with a side chain containing at least one aromatic or heteroaromatic ring, which may contain one or more substituents such as —OH, —SH, —CN, —F, —CI, —Br, —I, —NO₂, —NO, —NH₂, —NHR, —NRR, —C(O)R, —C(O)OH, —C(O)OR, —C(O)NH₂, —C(O)NHR, —C(O)NRR, etc., where R is independently (—C₆) alkyl, substituted (C_{Ï}-C₆) alkyl, (C C₆) alkenyl, substituted (—C₆) alkenyl, (Cj-C₆) alkynyl, substituted (Cι-C₆) alkynyl, (C₅-C₂o) aryl, substituted (C₅-C₂₀) aryl, (C₆-C₂₆) alkaryl, substituted (C₆-C₂₆) alkaryl, 5-20 membered heteroaryl, substituted 5-20 membered heteroaryl, 6-26 membered alkheteroaryl or substituted 6-26 membered alkheteroaryl. Genetically encoded aromatic amino acids include Phe, Tyr, and Trp, while non-genetically encoded aromatic amino acids include phenylglycine, 2-napthylalanine, beta-2-thienylalanine, 1,2,3,4-tetrahydro-isoquinoline-3-carboxylic acid, 4-chlorophenylalanine, 2-fluorophenylalanine3-fluorophenylalanine, and 4-fluorophenylalanine.

An apolar amino acid is a hydrophobic amino acid with a side chain that is uncharged at physiological pH and which has bonds in which a pair of electrons shared in common by two atoms is generally held equally by each of the two atoms (i.e., the side chain is not polar). Genetically encoded apolar amino acids include Gly, Leu, Val, He, Ala, and Met, while non-genetically encoded apolar amino acids include cyclohexylalanine. Apolar amino acids can be further subdivided to include aliphatic amino acids, which is a hydrophobic amino acid having an aliphatic hydrocarbon side chain. Genetically encoded aliphatic amino acids include Ala, Leu, Val, and He, while non-genetically encoded aliphatic amino acids include norleucine.

A polar amino acid is a hydrophilic amino acid with a side chain that is uncharged at physiological pH, but which has one bond in which the pair of electrons shared in common by two atoms is held more closely by one of the atoms.

Genetically encoded polar amino acids include Ser, Thr, Asn, and Gin, while non-genetically encoded polar amino acids include citrulline, N-acetyl lysine, and methionine sulfoxide.

An acidic amino acid is a hydrophilic amino acid with a side chain pKa value of less than 7. Acidic amino acids typically have negatively charged side chains at physiological pH due to loss of a hydrogen ion. Genetically encoded acidic amino acids include Asp and Glu. A basic amino acid is a hydrophilic amino acid with a side chain pKa value of greater than 7. Basic amino acids typically have positively charged side chains at physiological pH due to association with hydronium ion.

Genetically encoded basic amino acids include Arg, Lys, and His, while non-genetically encoded basic amino acids include the non-cyclic amino acids ornithine, 2,3,-diaminopropionic acid, 2,4-diaminobutyric acid, and homoarginine.
It will be appreciated by one skilled in the art that the above classifications are not absolute and that an amino acid may be classified in more than one category. In addition, amino acids can be classified based on known behaviour and or characteristic chemical, physical, or biological properties based on specified assays or as compared with previously identified amino acids. Amino acids can also include bifunctional moieties having amino acid-like side chains.

Conservative changes can also include the substitution of a chemically derivatised moiety for a non-derivatised residue, by for example, reaction of a functional side group of an amino acid. Thus, these substitutions can include compounds whose free amino groups have been derivatised to amine hydrochlorides, p-toluene sulfonyl groups, carbobenzoxy groups, t-butyloxycarbonyl groups, chloroacetyl groups or formyl groups. Similarly, free carboxyl groups can be derivatized to form salts, methyl and ethyl esters or other types of esters or hydrazides, and side chains can be derivatized to form O-acyl or O-alkyl derivatives for free hydroxyl groups or N-im-benzylhistidine for the imidazole nitrogen of histidine.

Peptides or peptide analogues can be synthesised by standard chemical techniques, for example, by automated synthesis using solution or solid phase synthesis methodology. Automated peptide synthesisers are commercially available and use techniques well known in the art. Peptides and peptide analogues can also be prepared using recombinant DNA technology using standard methods such as those described in, for example, Sambrook, et al. (Molecular Cloning: A Laboratory Manual. 3^{rd} ed., Cold Spring Harbor Laboratory, Cold Spring Harbor Laboratory Press, Cold Spring Harbor, N. Y., 2000) or Ausubel et al. (Current Protocols in Molecular Biology, John Wiley & Sons, New York, N.Y., 1987-2012).

Accordingly, and as discussed herein, compounds for use according to the disclosure include nucleic acid molecules encoding the fusion proteins described herein.

The terms “nucleic acid” or “nucleic acid molecule” encompass both RNA (plus and minus strands) and DNA, including cDNA, genomic DNA, and synthetic (e.g., chemically synthesized) DNA. The nucleic acid may be double-stranded or single-stranded. Where single-stranded, the nucleic acid may be the sense strand or the antisense strand. A nucleic acid molecule may be any chain of two or more covalently bonded nucleotides, including naturally occurring or non-naturally occurring nucleotides, or nucleotide analogs or derivatives. By “RNA” is meant a sequence of two or more covalently bonded, naturally occurring or modified ribonucleotides. One example of a modified RNA included within this term is phosphorothioate RNA. By “DNA” is meant a sequence of two or more covalently bonded, naturally occurring or modified deoxyribonucleotides. By “cDNA” is meant complementary or copy DNA produced from an RNA template by the action of RNA-dependent DNA polymerase (reverse transcriptase). Thus a “cDNA clone” means a duplex DNA sequence complementary to an RNA molecule of interest, carried in a cloning vector. By “complementary” is meant that two nucleic acids, e.g., DNA or RNA, contain a sufficient number of nucleotides which are capable of forming Watson-Crick base pairs to produce a region of double-strandedness between the two nucleic acids. Thus, adenine in one strand of DNA or RNA pairs with thymine in an opposing complementary DNA strand or with uracil in an opposing complementary RNA strand. It will be understood that each nucleotide in a nucleic acid molecule need not form a matched Watson-Crick base pair with a nucleotide in an opposing complementary strand to form a duplex. A nucleic acid molecule is “complementary” to another nucleic acid molecule if it hybridizes, under conditions of high stringency, with the second nucleic acid molecule.

A compound is “isolated” when it is separated from the components that naturally accompany it. Typically, a compound is isolated when it is at least 50%, or 60%, or more generally at least 70%, 75%, 80%, 85%, 90%, 95%, or 99% by weight, of the total material in a sample. Thus, for example, a polypeptide that is chemically synthesized or produced by recombinant technology will be generally be substantially free from its naturally associated components. A nucleic acid molecule will generally be substantially pure or “isolated” when it is not immediately contiguous with (i.e., covalently linked to) the coding sequences with which it is normally contiguous in the naturally occurring genome of the organism from which the DNA of the invention is derived. Therefore, an “isolated” gene or nucleic acid molecule is intended to mean a gene or nucleic acid molecule which is not flanked by nucleic acid molecules which normally (in nature) flank the gene or nucleic acid molecule (such as in genomic sequences) and/or has been completely or partially purified from other transcribed sequences (as in a cDNA or RNA library). For example, an isolated nucleic acid of the invention may be substantially isolated with respect to the complex cellular milieu in which it naturally occurs. The term therefore includes, e.g., a recombinant nucleic acid incorporated into a vector, such as an autonomously replicating plasmid or virus; or into the genomic DNA of a prokaryote or eukaryote, or which exists as a separate molecule (e.g., a cDNA or a genomic DNA fragment produced by PCR or restriction endonuclease treatment) independent of other sequences. It also includes a recombinant nucleic acid which is part of a hybrid gene encoding additional polypeptide sequences. Preferably, an isolated nucleic acid comprises at least about 50, 80 or 90 percent (on a molar basis) of all macromolecular species present. Thus, an isolated gene or nucleic acid molecule can include a gene or nucleic acid molecule which is synthesized chemically or by recombinant means. Recombinant DNA contained in a vector are included in the definition of “isolated” as used herein. Also, isolated nucleic acid molecules include recombinant DNA molecules in heterologous host cells, as well as partially or substantially purified DNA molecules in solution. In vivo and in vitro RNA transcripts of the DNA molecules of the present invention are also encompassed by “isolated” nucleic acid molecules.

Various genes and nucleic acid sequences of the invention may be recombinant sequences. The term “recombinant” means that something has been recombined, so that when made in reference to a nucleic acid construct the term refers to a molecule that is comprised of nucleic acid sequences that are joined together or produced by means of molecular biological techniques. The term “recombinant” when made in reference to a protein or a polypeptide refers to a protein or polypeptide molecule which is expressed using a recombinant nucleic acid construct created by means of molecular biological techniques. Recombinant nucleic acid constructs may include a nucleotide sequence which is ligated to, or is manipulated to become ligated to, a nucleic acid sequence to which it is not ligated in nature, or to which it is ligated at a different location in nature. Referring to a nucleic acid construct as “recombinant” therefore indicates that the nucleic acid molecule has been manipulated using genetic engineering, i.e. by human intervention.

Recombinant nucleic acid constructs may for example be introduced into a host cell by transformation. Such recombinant nucleic acid constructs may include sequences derived from the same host cell species or from different host cell species, which have been isolated and reintroduced into cells of the host species. Recombinant nucleic acid construct sequences may become integrated into a host cell genome, either as a result of the original transformation of the host cells, or as the result of subsequent recombination and/or repair events.

As used herein, “heterologous” in reference to a nucleic acid or protein is a molecule that has been manipulated by human intervention so that it is located in a place other than the place in which it is naturally found. For example, a nucleic acid sequence from one species may be introduced into the genome of another species, or a nucleic acid sequence from one genomic locus may be moved to another genomic or extrachromasomal locus in the same species. A heterologous protein includes, for example, a protein expressed from a heterologous coding sequence or a protein expressed from a recombinant gene in a cell that would not naturally express the protein. A heterologous fusion protein may include a protein in a non-natural orientation (i.e., N to C) or may include a fragment or portion of a protein located in a place, within the protein, other than the place in which it is naturally found.

A “substantially identical” sequence is an amino acid or nucleotide sequence that differs from a reference sequence only by one or more conservative substitutions, as discussed herein, or by one or more non-conservative substitutions, deletions, or insertions located at positions of the sequence that do not destroy the biological function of the amino acid or nucleic acid molecule. Such a sequence can be any integer from 45% to 99%, or more generally at least 45%, 50, 55% or 60%, or at least 65%, 75%, 80%, 85%, 90%, or 95%, or as much as 96%, 97%, 98%, or 99% identical at the amino acid or nucleotide level to the sequence used for comparison using, for example, the Align Program or FASTA. For polypeptides, the length of comparison sequences may be at least 2, 5, 10, or 15 amino acids, or at least 20, 25, or 30 amino acids. In alternate embodiments, the length of comparison sequences may be at least 35, 40, or 50 amino acids, or over 60, 80, or 100 amino acids. For nucleic acid molecules, the length of comparison sequences may be at least 5, 10, 15, 20, or 25 nucleotides, or at least 30, 40, or 50 nucleotides. In alternate embodiments, the length of comparison sequences may be at least 60, 70, 80, or 90 nucleotides, or over 100, 200, or 500 nucleotides. Sequence identity can be readily measured using publicly available sequence analysis software (e.g., Sequence Analysis Software Package of the Genetics Computer Group, University of Wisconsin Biotechnology Center, 1710 University Avenue, Madison, Wis. 53705, or BLAST software available from the National Library of Medicine, or as described herein). Examples of useful software include the programs Pile-up and PrettyBox. Such software matches similar sequences by assigning degrees of homology to various substitutions, deletions, substitutions, and other modifications.

Alternatively, or additionally, two nucleic acid sequences may be “substantially identical” if they hybridize under high stringency conditions. In some embodiments, high stringency conditions are, for example, conditions that allow hybridization comparable with the hybridization that occurs using a DNA probe of at least 500 nucleotides in length, in a buffer containing 0.5 M NaHPO₄, pH 7.2, 7% SDS, 1 mM EDTA, and 1% BSA (fraction V), at a temperature of 65° C., or a buffer containing 48% formamide, 4.8×SSC, 0.2 M Tris-C1, pH 7.6, 1×Denhardt's solution, 10% dextran sulfate, and 0.1% SDS, at a temperature of 42° C. (These are typical conditions for high stringency northern or Southern hybridizations.) Hybridizations may be carried out over a period of about 20 to 30 minutes, or about 2 to 6 hours, or about 10 to 15 hours, or over 24 hours or more. High stringency hybridization is also relied upon for the success of numerous techniques routinely performed by molecular biologists, such as high stringency PCR, DNA sequencing, single strand conformational polymorphism analysis, and in situ hybridization. In contrast to northern and Southern hybridizations, these techniques are usually performed with relatively short probes (e.g., usually about 16 nucleotides or longer for PCR or sequencing and about 40 nucleotides or longer for in situ hybridization). The high stringency conditions used in these techniques are well known to those skilled in the art of molecular biology (Ausubel et al, Current Protocols in Molecular Biology, John Wiley & Sons, New York, N.Y., 1998).

Substantially identical sequences may for example be sequences that are substantially identical to the Chlamydia spp. sequences described or referenced herein. A substantially identical sequence may for example be a sequence that is substantially identical to the sequence of any one of SEQ ID NOs: 1-71, or to any one of the sequences indicated by the locus tags referenced in the C. trachomatis genome sequence and/or the C. muridarum genome sequence as indicated herein, or a fragment or variant thereof. In some embodiments, a substantially identical sequence may for example be a nucleotide sequence that is complementary to or hybridizes with the sequence of any one of the nucleic acid sequences described herein, or with the sequence encoding any one of the amino acid sequences described herein, or to any one of the sequences indicated by the locus tags referenced in the C. trachomatis genome sequence and/or the C. muridarum genome sequence as indicated herein, or a fragment or variant thereof. In some embodiments, a substantially identical sequence may be derived from a Chlamydia spp., such as a C. trachomatis or a C. muridarum.

Pharmaceutical & Veterinary Compositions, Dosages, and Administration

The compounds and compositions as described herein may be used to prepare vaccine or other formulations and/or used in the induction of an immune response to a Chlamydia antigen or epitope. In some embodiments, the compositions may be formulated as admixtures of fusion proteins consisting of two or more Chlamydia proteins or immunogenic fragments thereof, as described herein. In alternative embodiments, the compositions may be formulated using a single fusion protein. In alternative embodiments, the compositions may include MOMP, either as part of a fusion protein or as an individual protein in admixture with a fusion protein as described herein.

The compounds and compositions can be provided alone or in combination with other compounds (for example, nucleic acid molecules, small molecules, polypeptides, peptides, or peptide analogues), in the presence of a liposome, an adjuvant, or any pharmaceutically acceptable carrier, in a form suitable for administration to an animal subject, for example, mice, humans, pigs, etc. If desired, treatment with a compound according to the invention may be combined with more traditional and existing therapies for Chlamydia infection.

Conventional pharmaceutical practice may be employed to provide suitable formulations to administer the compounds or compositions to subjects infected by a Chlamydia pathogen. Any appropriate route of administration may be employed, for example, parenteral, intravenous, subcutaneous, intramuscular, intracranial, intrathecal, intraorbital, ophthalmic, intraventricular, intracapsular, intraspinal, intracisternal, intraperitoneal, intranasal, epidermal, transdermal, mucosal membrane aerosol, nasal, rectal, vaginal, topical or oral administration. In some embodiments, the compounds or compositions described herein may be applied to epithelial surfaces. Some epithelial surfaces may comprise a mucosal membrane, for example buccal, gingival, nasal, tracheal, bronchial, gastrointestinal, genital, rectal, urethral, vaginal, cervical, uterine and the like. Some epithelial surfaces may comprise keratinized cells, for example, skin, tongue, gingival, palate or the like. In some embodiments, the Chlamydia infection may be in the lung, genital tract or eye and the compounds or compositions described herein may be administered intranasally or by injection.

Formulations may be in the form of liquid solutions or suspensions; tablets or capsules; powders, nasal drops, or aerosols. Methods are well known in the art for making formulations (Berge et al. 1977. J. Pharm Sci. 66: 1-19); Remington—The Science and Practice of Pharmacy, 21^{st} edition. Gennaro et al editors. Lippincott Williams & Wilkins Philadelphia.). Such excipients may include, for example, salts, buffers, antioxidants, complexing agents, tonicity agents, cryoprotectants, lyoprotectants, suspending agents, emulsifying agents, antimicrobial agents, preservatives, chelating agents, binding agents, surfactants, wetting agents, anti-adherents agents, disentegrants, coatings, glidants, deflocculating agents, anti-nucleating agents, surfactants, stabilizing agents, non-aqueous vehicles such as fixed oils, polymers or encapsulants for sustained or controlled release, ointment bases, fatty acids, cream bases, emollients, emulsifiers, thickeners, preservatives, solubilizing agents, humectants, water, alcohols or the like.

Formulations for parenteral administration may, for example, contain excipients, sterile water, or saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds or compositions. Other potentially useful parenteral delivery systems for modulatory compounds include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops, or as a gel.

For therapeutic or prophylactic compositions, the compounds or compositions are administered to an animal in an amount effective to stop or slow a Chlamydia infection.

An “effective amount” of a compound according to the invention includes a therapeutically effective amount or a prophylactically effective amount. A “therapeutically effective amount” refers to an amount effective, at dosages and for periods of time necessary, to achieve the desired therapeutic result, such as reduction of a Chlamydia infection or induction of an immune response to a Chlamydia antigen or epitope. A therapeutically effective amount of a compound may vary according to factors such as the disease state, age, sex, and weight of the subject, and the ability of the compound to elicit a desired response in the subject. Dosage regimens may be adjusted to provide the optimum therapeutic response. A therapeutically effective amount is also one in which any toxic or detrimental effects of the compound are outweighed by the therapeutically beneficial effects. A “prophylactically effective amount” refers to an amount effective, at dosages and for periods of time necessary, to achieve the desired prophylactic result, such as prevention of a Chlamydia infection or induction of an immune response to a Chlamydia antigen or epitope. Typically, a prophylactic dose is used in subjects prior to or at an earlier stage of disease, so that a prophylactically effective amount may be less than a therapeutically effective amount. A suitable range for therapeutically or prophylactically effective amounts of a compound maybe any integer from 0.1 nM-0.1M, 0.1 nM-0.05M, 0.05 nM-15 μM or 0.01 nM-10 μM.

In some embodiments, an effective amount may be calculated on a mass/mass basis (e.g. micrograms or milligrams per kilogram of subject), or may be calculated on a mass/volume basis (e.g. concentration, micrograms or milligrams per milliliter). Using a mass/volume unit, one or more peptides or polypeptides may be present at an amount from about 0.1 ug/ml to about 20 mg/ml, or any amount therebetween, for example 0.1, 0.5, 1, 2, 5, 10, 15, 20, 25, 30, 35, 40, 50, 60, 70, 80, 90, 100, 120, 140, 160 180, 200, 250, 500, 750, 1000, 1500, 2000, 5000, 10000, 20000 ug/ml, or any amount therebetween; or from about 1 ug/ml to about 2000 ug/ml, or any amount therebetween, for example 1.0, 2.0, 5.0, 10.0, 15.0, 20.0, 25.0, 30.0, 35.0, 40.0, 50.0 60.0, 70.0, 80.0, 90.0, 100, 120, 140, 160 180, 200, 250, 500, 750, 1000, 1500, 2000, ug/ml or any amount therebetween; or from about 10 ug/ml to about 1000 ug/ml or any amount therebetween, for example 10.0, 15.0, 20.0, 25.0, 30.0, 35.0, 40.0, 50.0 60.0, 70.0, 80.0, 90.0, 100, 120, 140, 160 180, 200, 250, 500, 750, 1000 ug/ml, or any amount therebetween; or from about 30 ug/ml to about 1000 ug/ml or any amount therebetween, for example 30.0, 35.0, 40.0, 50.0 60.0, 70.0, 80.0, 90.0, 100, 120, 140, 160 180, 200, 250, 500, 750, 1000 ug/ml.

Quantities and/or concentrations may be calculated on a mass/mass basis (e.g. micrograms or milligrams per kilogram of subject), or may be calculated on a mass/volume basis (e.g. concentration, micrograms or milligrams per milliliter). Using a mass/volume unit, one or more peptides or polypeptides may be present at an amount from about 0.1 ug/ml to about 20 mg/ml, or any amount therebetween, for example 0.1, 0.5, 1, 2, 5, 10, 15, 20, 25, 30, 35, 40, 50, 60, 70, 80, 90, 100, 120, 140, 160 180, 200, 250, 500, 750, 1000, 1500, 2000, 5000, 10000, 20000 ug/ml, or any amount therebetween; or from about 1 ug/ml to about 2000 ug/ml, or any amount therebetween, for example 1.0, 2.0, 5.0, 10.0, 15.0, 20.0, 25.0, 30.0, 35.0, 40.0, 50.0 60.0, 70.0, 80.0, 90.0, 100, 120, 140, 160 180, 200, 250, 500, 750, 1000, 1500, 2000, ug/ml or any amount therebetween; or from about 10 ug/ml to about 1000 ug/ml or any amount therebetween, for example 10.0, 15.0, 20.0, 25.0, 30.0, 35.0, 40.0, 50.0 60.0, 70.0, 80.0, 90.0, 100, 120, 140, 160 180, 200, 250, 500, 750, 1000 ug/ml, or any amount therebetween; or from about 30 ug/ml to about 1000 ug/ml or any amount therebetween, for example 30.0, 35.0, 40.0, 50.0 60.0, 70.0, 80.0, 90.0, 100, 120, 140, 160 180, 200, 250, 500, 750, 1000 ug/ml.

Compositions according to various embodiments of the invention, including therapeutic compositions, may be administered as a dose comprising an effective amount of one or more peptides or polypeptides. The dose may comprise from about 0.1 ug/kg to about 20 mg/kg (based on the mass of the subject), for example 0.1, 0.5, 1, 2, 5, 10, 15, 20, 25, 30, 35, 40, 50, 60, 70, 80, 90, 100, 120, 140, 160 180, 200, 250, 500, 750, 1000, 1500, 2000, 5000, 10000, 20000 ug/kg, or any amount therebetween; or from about 1 ug/kg to about 2000 ug/kg or any amount therebetween, for example 1.0, 2.0, 5.0, 10.0, 15.0, 20.0, 25.0, 30.0, 35.0, 40.0, 50.0 60.0, 70.0, 80.0, 90.0, 100, 120, 140, 160 180, 200, 250, 500, 750, 1000, 1500, 2000 ug/kg, or any amount therebetween; or from about 10 ug/kg to about 1000 ug/kg or any amount therebetween, for example 10.0, 15.0, 20.0, 25.0, 30.0, 35.0, 40.0, 50.0 60.0, 70.0, 80.0, 90.0, 100, 120, 140, 160 180, 200, 250, 500, 750, 1000 ug/kg, or any amount therebetween; or from about 30 ug/kg to about 1000 ug/kg or any amount therebetween, for example 30.0, 35.0, 40.0, 50.0 60.0, 70.0, 80.0, 90.0, 100, 120, 140, 160 180, 200, 250, 500, 750, 1000 ug/kg.

One of skill in the art will be readily able to interconvert the units as necessary, given the mass of the subject, the concentration of the composition, individual components or combinations thereof, or volume of the composition, individual components or combinations thereof, into a format suitable for the desired application.

It is to be noted that dosage values may vary with the severity of the condition to be alleviated. For any particular subject, specific dosage regimens may be adjusted over time according to the individual need and the professional judgment of the person administering or supervising the administration of the compositions. Dosage ranges set forth herein are exemplary only and do not limit the dosage ranges that may be selected by medical practitioners. The amount of active compound in the composition may vary according to factors such as the disease state, age, sex, and weight of the individual. Dosage regimens may be adjusted to provide the optimum therapeutic response. For example, a single bolus may be administered, several divided doses may be administered over time or the dose may be proportionally reduced or increased as indicated by the exigencies of the therapeutic situation. It may be advantageous to formulate parenteral compositions in dosage unit form for ease of administration and uniformity of dosage.

The amount of a composition administered, where it is administered, the method of administration and the timeframe over which it is administered may all contribute to the observed effect. As an example, a composition may be administered systemically e.g. by intravenous administration and have a toxic or undesirable effect, while the same composition administered subcutaneously or intranasally may not yield the same undesirable effect. In some embodiments, localized stimulation of immune cells in the lymph nodes close to the site of subcutaneous injection may be advantageous, while a systemic immune stimulation may not.

In general, compounds or compositions should be used without causing substantial toxicity. Toxicity of the compounds of the invention can be determined using standard techniques, for example, by testing in cell cultures or experimental animals and determining the therapeutic index, i.e., the ratio between the LD50 (the dose lethal to 50% of the population) and the LD100 (the dose lethal to 100% of the population). In some circumstances however, such as in severe disease conditions, it may be necessary to administer substantial excesses of the compositions.

Compositions according to various embodiments of the invention may be provided in a unit dosage form, or in a bulk form suitable for formulation or dilution at the point of use. Compositions according to various embodiments of the invention may be administered to a subject in a single-dose, or in several doses administered over time. Dosage schedules may be dependent on, for example, the subject's condition, age, gender, weight, route of administration, formulation, or general health. Dosage schedules may be calculated from measurements of adsorption, distribution, metabolism, excretion and toxicity in a subject, or may be extrapolated from measurements on an experimental animal, such as a rat or mouse, for use in a human subject. Optimization of dosage and treatment regimens are discussed in, for example, Goodman & Gilman's The Pharmacological Basis of Therapeutics 11^{th} edition. 2006. LL Brunton, editor. McGraw-Hill, New York, or Remington—The Science and Practice of Pharmacy, 21^{st} edition. Gennaro et al editors. Lippincott Williams & Wilkins Philadelphia.

A “vaccine” is a composition that includes materials that elicit a desired immune response. A desired immune response may include protection against infection by a Chlamydia spp. pathogen. For example, a desired immune response may include any value from between 10% to 100%, e.g., 10%, 20%, 30%, 40%, 50%, 60%, 70%, 80%, 90%, 100%, protection against infection by a Chlamydia spp. pathogen in a vaccinated animal when compared to a non-vaccinated animal.

An “immune response” may generally refer to a response of the adaptive immune system, such as a humoral response, and a cell-mediated response. The humoral response is the aspect of immunity that is mediated by secreted antibodies, produced in the cells of the B lymphocyte lineage (B cell). Secreted antibodies bind to antigens on the surfaces of invading microbes (such as viruses or bacteria), which flags them for destruction. Humoral immunity is used generally to refer to antibody production and the processes that accompany it, as well as the effector functions of antibodies, including Th2 cell activation and cytokine production, memory cell generation, opsonin promotion of phagocytosis, pathogen elimination and the like. A cell-mediated response may refer to an immune response that does not involve antibodies but rather involves the activation of macrophages, natural killer cells (NK), antigen-specific cytotoxic T-lymphocytes, and the release of various cytokines in response to an antigen. Cell-mediated immunity may generally refer to some Th cell activation, Tc cell activation and T-cell mediated responses.

Antigen presenting cells (APCs) such as dendritic cells (DCs) take up polypeptides and present epitopes of such polypeptides within the context of the DC MHC I and II complexes to other immune cells including CD4+ and CD8+ cells. An “MHC complex” or “MHC receptor” is a cell-surface receptor encoded by the major histocompatibility complex of a subject, with a role in antigen presentation for the immune system. MHC proteins may be found on several cell types, including antigen presenting cells (APCs) such as macrophages or dendritic cells (DCs), or other cells found in a mammal. Epitopes associated with MHC Class I may range from about 8-11 amino acids in length, while epitopes associated MHC Class II may be longer, ranging from about 9-25 amino acids in length.

Accordingly, an “immune response” includes, but is not limited to, one or more of the following responses in a mammal: induction of antibodies, B cells, T cells (including helper T cells, suppressor T cells, cytotoxic T cells, γδ T cells) directed specifically to the antigen(s) in a composition or vaccine, following administration of the composition or vaccine. An immune response to a composition or vaccine thus generally includes the development in the host mammal of a cellular and/or antibody-mediated response to the composition or vaccine of interest. In general, the immune response will result in prevention or reduction of infection by a Chlamydia spp. pathogen. In some embodiments, an immune response refers specifically to a cell-mediated response. In some embodiments, an immune response refers specifically to a cell-mediated response against a Chlamydia spp. pathogen. In some embodiments, the compounds and compositions described herein may be used in the induction of a cell-mediated immune response against a Chlamydia spp. pathogen.

Vaccines according to the disclosure may include the polypeptides and nucleic acid molecules described herein, or immunogenic fragments thereof, and may be administered using any form of administration known in the art or described herein.

An “immunogenic fragment” of a polypeptide or nucleic acid molecule refers to an epitope or amino acid or nucleotide sequence that elicits an immune response. The term “epitope” refers to an arrangement of amino acids in a protein or modifications thereon (for example glycosylation). The amino acids may be arranged in a linear fashion, such as a primary sequence of a protein, or may be a secondary or tertiary arrangement of amino acids in close proximity once a protein is partially or fully configured. Epitopes may be specifically bound by an antibody, antibody fragment, peptide, peptidomimetic or the like, or may be specifically bound by a ligand or held within an MHC I or MHC II complex. Epitopes may be present in a larger fragment or sequence of a Chlamydia protein as described herein.

Thus, an immunogenic fragment may include, without limitation, any portion of any of the sequences described herein, or a sequence substantially identical thereto, that includes one or more epitopes (the site recognized by a specific immune system cell, such as a T cell). For example, an immunogenic fragment may include, without limitation, peptides of any value between 6 and 60, or over 60, amino acids in length, e.g., peptides of any value between 10 and 20 amino acids in length, or between 20 and 40 amino acids in length, derived from any one or more of the sequences described herein. Such fragments may be identified using standard methods known to those of skill in the art, such as epitope mapping techniques or antigenicity or hydropathy plots using, for example, the Omiga version 1.0 program from Oxford Molecular Group (see, for example, U.S. Pat. No. 4,708,871)(76, 77, 81, 92, 73,). An epitope may have a range of sizes—for example a linear epitope may be as small as two amino acids, or may be larger, from about 3 amino acids to about 20 amino acids. In some embodiments, an epitope may be from about 5 amino acids to about 10 or about 15 amino acids in length. An epitope of secondary or tertiary arrangements of amino acids may encompass as few as two amino acids, or may be larger, from about 3 amino acids to about 20 amino acids. In some embodiments, a secondary or tertiary epitope may be from about 5 amino acids to about 10 or about 15 amino acids in proximity to some or others within the epitope. In some embodiments, a fusion protein as described herein will contain multiple epitopes; in such cases, an immunogenic fragment may include a significant portion of a whole protein that is present in a fusion protein, as described herein.

In some embodiments, a vaccine includes a suitable carrier, such as an adjuvant, which is an agent that acts in a non-specific manner to increase the immune response to a specific antigen, or to a group of antigens, enabling the reduction of the quantity of antigen in any given vaccine dose, or the reduction of the frequency of dosage required to generate the desired immune response.

Exemplary adjuvants include, without limitation, aluminum hydroxide, alum, Alhydrogel™ (aluminum trihydrate) or other aluminum-comprising salts, virosomes, nucleic acids comprising CpG motifs such as CpG oligodeoxynucleotides (CpG-ODN), squalene, oils, MF59 (Novartis), LTK63 (Novartis), QS21, various saponins, virus-like particles, monomycolyl glycerol (MMG), monophosphoryl-lipid A (MPL)/trehalose dicorynomycolate, toll-like receptor agonists, copolymers such as polyoxypropylene and polyoxyethylene, AbISCO, ISCOM (AbISCO-100), montanide ISA 51, Montanide ISA 720+CpG, etc. or any combination thereof. In some embodiments, exemplary adjuvants include a cationic lipid delivery agent such as dimethyldioctadecylammonium Bromide (DDA) together with a modified mycobacterial cord factor trehalose 6,6′-dibehenate (TDB) (DDA/TDB), DDA/MMG or DDA/MPL or any combination thereof. Liposomes with or without incorporated MPL further been adsorbed to alum hydroxide may also be useful, see, for example U.S. Pat. Nos. 6,093,406 and 6,793,923 B2. In some embodiments, exemplary adjuvants include prokaryotic RNA. In some embodiments, exemplary adjuvants include those described in for example US Patent Publication 2006/0286128 In some embodiments, exemplary adjuvants include DDA/TDB, DDA/MMG or DDA/MPL and prokaryotic RNA.

In some embodiments, vaccine compositions include, without limitation, fusion proteins as described herein in combination with DDA/TDB, DDA/MMG or DDA/MPL and, optionally, prokaryotic RNA.

In alternative embodiments, vaccine compositions include, without limitation, fusion proteins as described herein, in admixture with MOMP, in combination with DDA/TDB, DDA/MMG or DDA/MPL and, optionally, prokaryotic RNA.

In alternative embodiments, vaccine compositions include, without limitation, fusion proteins as described herein, in admixture with MOMP, in combination with DDA/TDB, DDA/MMG or DDA/MPL and, optionally, prokaryotic RNA.

In alternative embodiments, vaccine compositions include a) a recombinant fusion protein including the polypeptide sequences of the Chlamydia proteins PmpG, PmpE, PmpF and PmpH or immunogenic fragment thereof, b) the adjuvant DDA/MPL and c) prokaryotic RNA.

In alternative embodiments, vaccine compositions include a) a recombinant fusion protein including the polypeptide sequences of the Chlamydia proteins PmpG, PmpE, PmpF and PmpH or immunogenic fragment thereof, b) the adjuvant DDA/TDB and c) prokaryotic RNA.

In alternative embodiments, vaccine compositions include a) a recombinant fusion protein including the polypeptide sequences of the Chlamydia proteins PmpG, PmpE, PmpF and PmpH or immunogenic fragment thereof and b) the adjuvant DDA/MPL.

In alternative embodiments, vaccine compositions include a) a recombinant fusion protein including the polypeptide sequences of the Chlamydia proteins PmpG, PmpE, PmpF and PmpH and b) the adjuvant DDA/TDB.

In alternative embodiments, vaccine compositions include a formulation comprising a) a combination (admixture) of two separate fusion proteins, such as PmpG/PmpH and PmpE/PmpF respectively, or immunogenic fragments thereof; b) the adjuvant DDA/MPL and c) prokaryotic RNA.

In alternative embodiments, vaccine compositions include a formulation comprising a) a combination (admixture) of two separate fusion proteins, between PmpG/PmpH and PmpE/PmpF respectively, or immunogenic fragments thereof; b) the adjuvant DDA/TDB and c) prokaryotic RNA.

In alternative embodiments, vaccine compositions include a formulation comprising a) a combination (admixture) of two separate fusion proteins, between PmpG/PmpH and PmpE/PmpF respectively or immunogenic fragments thereof; and b) the adjuvant DDA/MPL.

In alternative embodiments, vaccine compositions include a formulation comprising a) a combination (admixture) of two separate fusion proteins, between PmpG/PmpH and PmpE/PmpF respectively or immunogenic fragments thereof; and b) the adjuvant DDA/TDB.

In some embodiments, a composition as described herein may be used to inoculate a test subject, for example, an animal model of Chlamydia infection, such as a mouse. Methods of experimentally inoculating experimental animals are known in the art. For example, testing a Chlamydia spp. vaccine may involve infecting previously inoculated mice intranasally with an inoculum comprising an infectious Chlamydia strain, and assessing for development of pneumonia. An exemplary assay is described in, for example Tammiruusu et al 2007. Vaccine 25(2):283-290, or in Rey-Ladino et al 2005. Infection and Immunity 73:1568-1577. It is within the ability of one of skill in the art to make any minor modifications to adapt such an assay to a particular pathogen model.

In another example, testing a Chlamydia vaccine may involve serially inoculating female mice with a candidate T-cell antigen cloned and expressed as described above. A series of inoculations may comprise two, three or more serial inoculations. The candidate T-cell antigens may be combined with an adjuvant. About three weeks following the last inoculation in the series, mice may be treated subcutaneously with 2.5 mg Depo-Provera and one week later both naive and immunized mice may be infected intravaginally with Chlamydia. The course of infection may be followed by monitoring the number of organisms shed at 2 to 7 day intervals for 6 weeks. The amount of organism shed may be determined by counting Chlamydia inclusion formation in HeLa cells using appropriately diluted vaginal wash samples. Immunity may be measured by the reduction in the amount of organism shed in immunized mice compared to naïve mice.

In some embodiments, the present disclosure also provides for a composition for inducing an immune response in a subject. Compositions according to various embodiments of the invention may be used as a vaccine, or in the preparation of a vaccine.

In another embodiment, a fusion protein as described herein may be used in the preparation of a medicament such as a vaccine composition, for the prevention or treatment of a Chlamydia infection. Treatment or treating includes prevention unless prevention is specifically excluded, as in alternative embodiments of the disclosure. Treatment or treating refers to fully or partially reducing severity of a Chlamydia infection and/or delaying onset of a Chlamydia infection, and/or reducing incidence of one or more symptoms or features of a Chlamydia infection, including reducing survival, growth, and/or spread of a Chlamydia spp., such as C. muridarum or C. trachomatis. In some embodiments, treatment includes inducing immunity in an animal subject. In alternative embodiments, treatment includes inducing cellular immunity in an animal subject. Treatment may be administered to a subject who does not exhibit signs of a disease, disorder, and/or condition (an asymptomatic subject), and/or to a subject who exhibits only early signs of a disease, disorder, and/or condition for the purpose of decreasing the risk of developing pathology associated with the disease, disorder, and/or condition. In some embodiments, treatment includes delivery of an immunogenic composition (e.g., a vaccine) to a subject.

The composition or medicament may be used for the prevention or treatment of a Chlamydia infection in a subject having, or suspected of having such an infection. In some embodiments, the composition or medicament may be used for the prevention or treatment of urogenital or ocular conditions. Urogenital conditions include without limitation urethritis, cervicitis, pharyngitis, proctitis, epididymitis, and prostatis. Ocular conditions include without limitation trachoma and conjunctivitis.

In some embodiments, a fusion protein described herein, alone or in combination, may be used to diagnose the presence of a Chlamydia infection in a subject for example even in an asymptomatic subject. Diagnosis may be determine T cell responses and may be performed using any technique described herein or known to the skilled person.

Articles of Manufacture

Also provided is an article of manufacture, comprising packaging material and a composition comprising one or more fusion proteins as provided herein. The composition includes a physiologically or pharmaceutically acceptable excipient, and may further include an adjuvant, a delivery agent, or an adjuvant and a delivery agent, and the packaging material may include a label which indicates the active ingredients of the composition (e.g. the fusion protein, adjuvant or delivery agent as present). The label may further include an intended use of the composition, for example as a therapeutic or prophylactic composition to be used in the manner described herein.

Kits

In another embodiment, a kit for the preparation of a medicament, comprising a composition comprising one or more fusion proteins as provided herein, along with instructions for its use is provided. The instructions may comprise a series of steps for the preparation of the medicament, the medicament being useful for inducing a therapeutic or prophylactic immune response in a subject to whom it is administered. The kit may further comprise instructions for use of the medicament in treatment for treatment, prevention or amelioration of one or more symptoms of a Chlamydia infection, and include, for example, dose concentrations, dose intervals, preferred administration methods or the like.

The present invention will be further illustrated in the following examples.

### EXAMPLES

### Example 1

### Molecular Cloning, Expression and Purification of Recombinant Fusion Proteins

PmpE, pmpF, pmpG, and pmpH DNA fragments were generated by PCR using genomic DNA isolated from C. muridarum. The DNA fragments generated by PCR were cloned stepwise into pET32a expression vector (GE Healthcare) after restriction enzyme digestion using standard molecular biology techniques. For all four pmp genes, only the regions that encode passenger domain of the Pmp protein were cloned into the vector for expression. The amino acid sequences of C. muridarum PmpE, PmpF, PmpG and PmpH proteins are shown in FIG. 1. Passenger domain portions of PmpE, PmpF, PmpG and PmpH, between amino acid 18 to 575, 20 to 575, 25 to 555, and 27 to 575, respectively, of the whole proteins were used. A C-terminal His-tag was introduced to all the fusion proteins. Plasmids containing the pmp genes were transformed into the E. coli strain BL21(DE3) (Strategene) where protein expression was carried out by inducing the lac promoter for expression of T7 RNA polymerase using isopropyl-b-D-thiogalactoside pyranoside.

The soluble expressed fusion proteins were purified from E. coli lysates by affinity chromatography using glutathione sepharose 4 fastflow purification system (GE Healthcare) using the N-terminal GST-tag. Insoluble fusion proteins were purified by nickel column using the His bind purification system (Qiagen) using the C-terminal His-tag and refolded by removing urea stepwise. LPS removal of these proteins was carried out by adding 0.1% Triton-114 in one of the wash buffers during purification. The amino acid sequences of recombinant fusion proteins between PmpE and PmpF (i.e. PmpE-PmpF) and PmpG and PmpH (i.e. PmpG-PmpH) are shown in FIG. 2.

Protection against Chlamydia genital tract infection in mice immunized with both individual protein/antigens, as well as in combinations of proteins formulated with different adjuvants, was evaluated. More specifically, groups of eight C57BL/6 mice were immunized 3 times at 2-week interval with Chlamydia proteins PmpG (G), PmpF (F), MOMP (M), either mixed or fused formulated with DDA/TDB (D/T) adjuvant. A group of mice immunized with phosphate buffered saline (PBS) was used as a negative control. Another group of mice infected once with 1,500 inclusion-forming units (IFU) of live C. muridarum elementary bodies (EB) intranasally (in) was used as a positive control. The experimental groups were as follows: 1) PBS (negative control), 2) PmpG+PmpF mixed proteins+DDA/TDB, 3) PmpG-PmpF fusion protein+DDA/TDB, 4) PmpG+MOMP mixed proteins+DDA/TDB, 5) PmpG-MOMP fusion protein+DDA/TDB and 6) Live EB (in) 1500 IFU (positive control). All groups were intravaginally challenged with 1,500 IFU of live C. muridarum EBs 4 weeks after the final immunization or 8 weeks after infection. Cervicovaginal washes were taken at day 6 and day 12, and bacterial titers were measured on HeLa 229 cells.

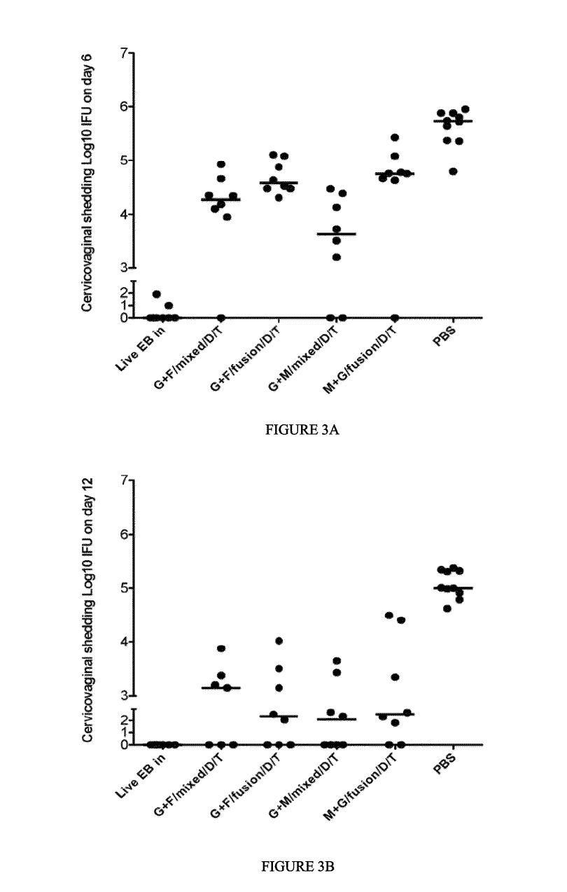

The results indicated that PmpG-PmpF and PmpG-MOMP fusion proteins formulated in the adjuvant DDA/TDB were protective against Chlamydia genital tract infection when evaluated at days 6 and 12 (FIGS. 3A-B).

### Example 2

To evaluate protective effect against Chlamydia muridarum genital tract infection, C57, Balb/c or C3H mice were immunized with PmpE, F, G, H plus MOMP (either individual proteins or fusion proteins), as in the following groups.

C57 mice: (1) PmpE+PmpF+PmpG+PmpH+MOMP (mixed); (2) PmpE-F fusion+PmpG-H fusion+MOMP (fusion); (3) PBS; (4) Live EB.

Balb/c mice: (5) PmpE+PmpF+PmpG+PmpH+MOMP (mixed); (6) PmpE-F fusion+PmpG-H fusion+MOMP (fusion); (7) PBS; (8) Live EB.

C3H mice: (9) PmpE+PmpF+PmpG+PmpH+MOMP (mixed); (10) PmpE-F fusion+PmpG-H fusion+MOMP (fusion); (11) PBS; (12) Live EB.

Mice were immunized 3 times at 2-week intervals with the fused proteins formulated with DDA/MPL. PBS was used as the negative control and mice infected once with 1,500 inclusion-forming units (IFU) of live C. muridarum elementary bodies (EB), administered intranasally, were used as positive controls. All groups were intravaginally challenged with 1,500 IFU of live C. muridarum elementary bodies 4 weeks after the final immunization or 8 weeks after live Chlamydia infection. Cervicovaginal washes were taken at day 12 and bacterial titers were measured on HeLa 229 cells to assess protection.

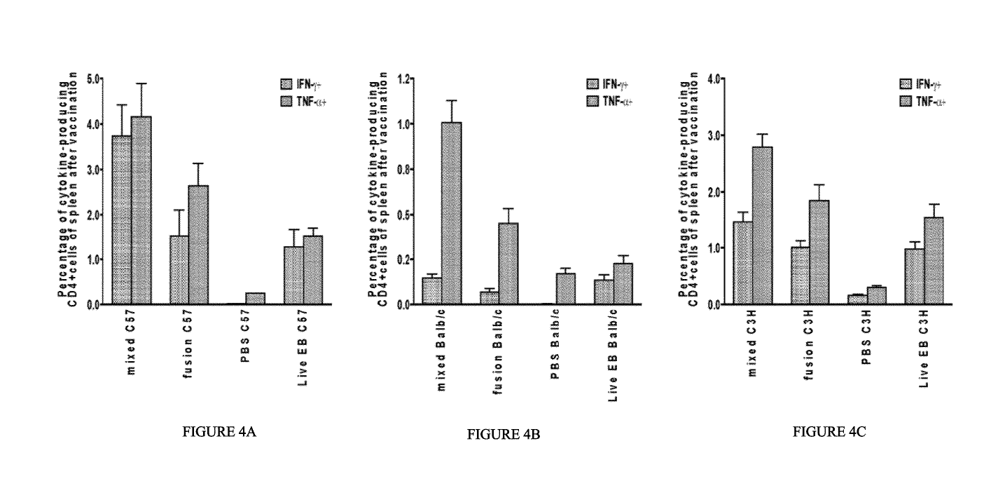

Two weeks after the final immunization, mouse splenocytes were harvested and stimulated with HK-EB (5×10⁵ IFU/ml). IFN-γ- or TNF-α-producing CD4 T cells were analyzed by multiparameter flow cytometry. The results are expressed as means±SEM for groups of four mice (FIG. 4).

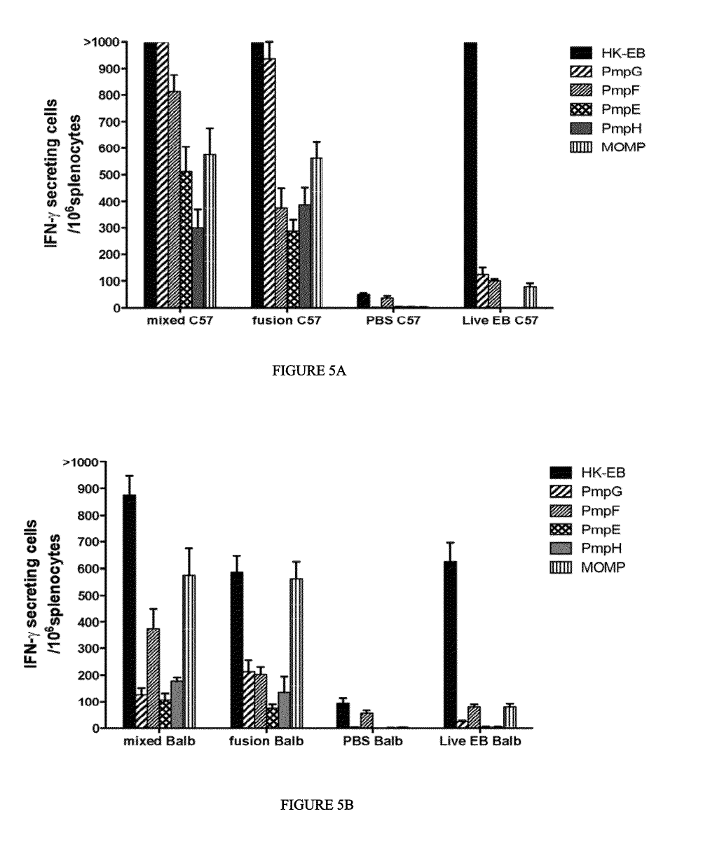

C. muridarum individual antigen-specific IFN-γ responses in C57, Balb/c, or C3H mice after immunization with PmpE, F, G, H plus MOMP either as individual (mixed) or as fusion formats were determined by ELISPOT assay. Two weeks after the final immunization, mouse splenocytes were harvested and stimulated in vitro for 20 h with 5×10⁵ IFU/ml of HK-EB, or 1 μg/ml of indicated Chlamydia recombinant protein respectively. The results are expressed as means±SEM for groups of four mice (FIGS. 5A-C).

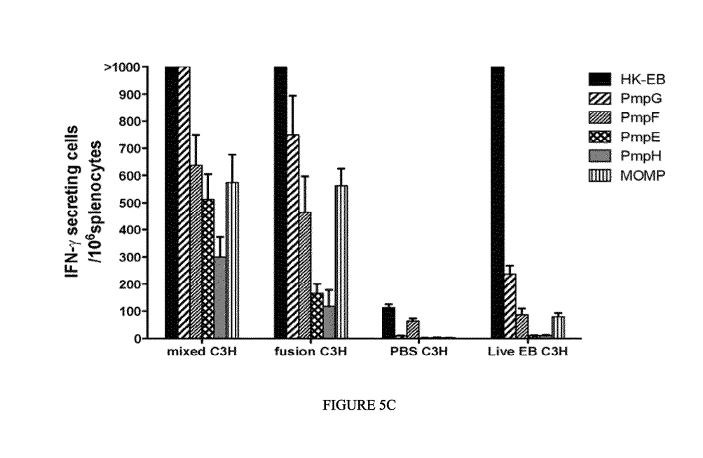

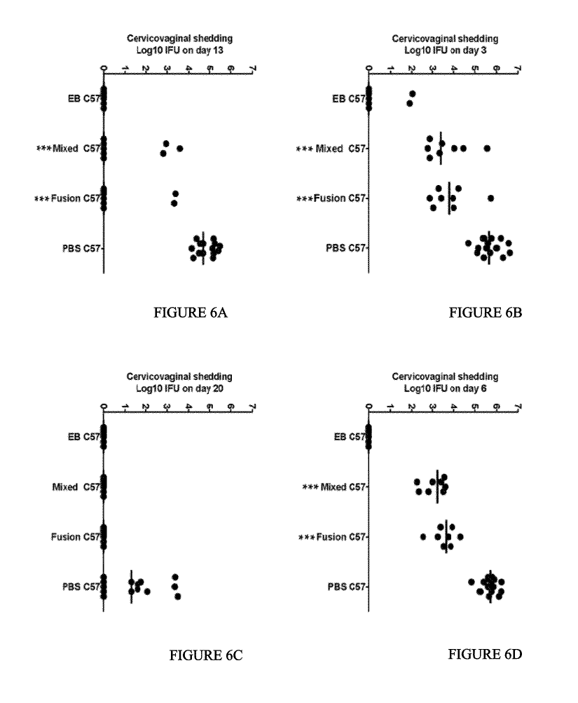

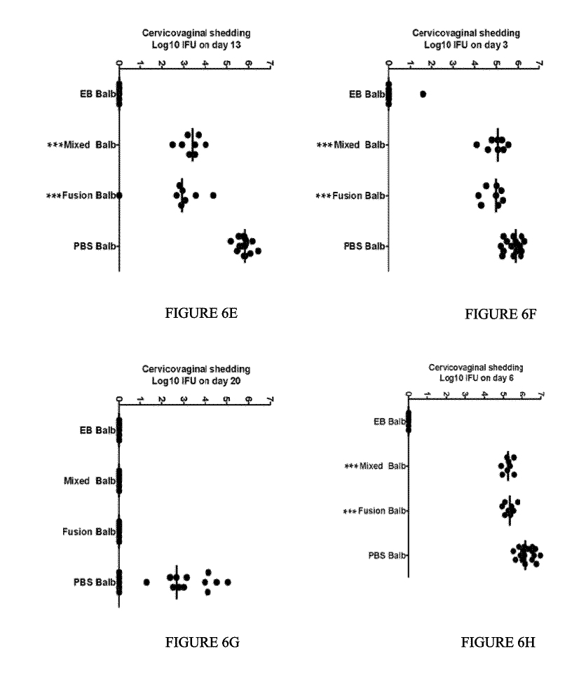

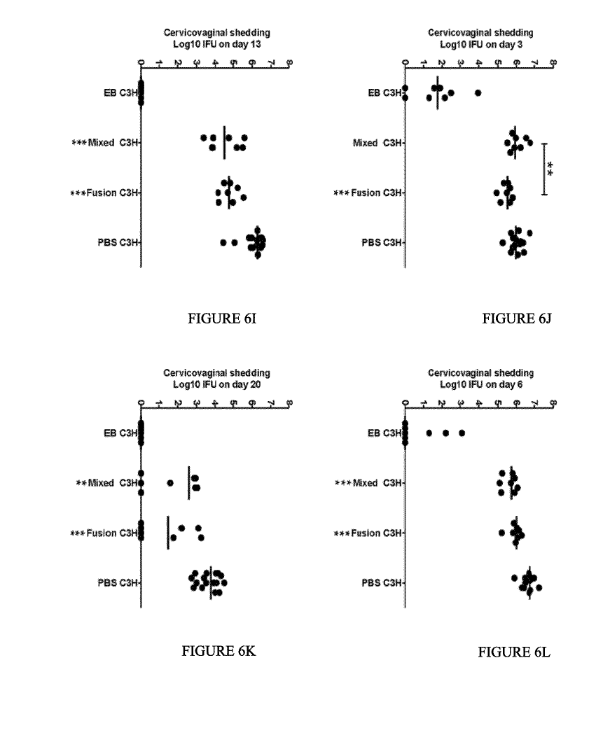

Vaccine-elicited protection against Chlamydia muridarum genital tract infection in C57, Balb/c, or C3H mice after immunization with PmpE, F, G, H plus MOMP, either as individual (mixed) or as fusion formats, was evaluated. Four weeks after the final immunization, mice were challenged intravaginally with 1,500 IFU of C. muridarum. Cervicovaginal washes were taken at day 3, day 6, day 13 and day 13 after infection, and bacterial shedding was measured on HeLa 229 cells. Mice immunized with PBS were used as a negative control, and mice infected with 1,500 IFU of live C. muridarum intravaginally were used as a positive control. ***P value<0.001 in comparison with the PBS group (FIGS. 6A-L).

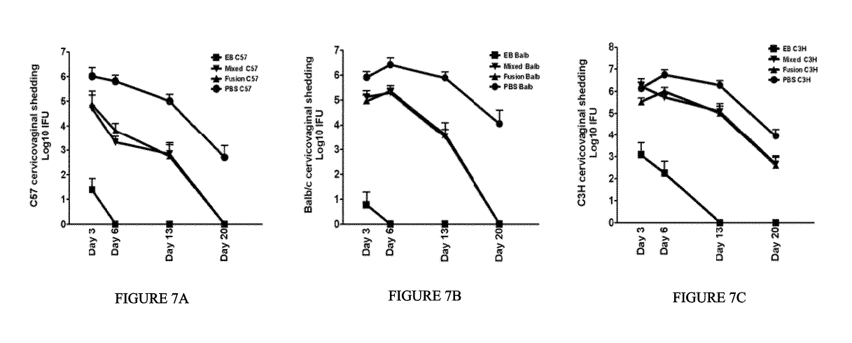

Vaccine-elicited protection against Chlamydia muridarum genital tract infection in C57 (A), Balb/c (B), or C3H (C) mice after immunization with PmpE, F, G, H plus MOMP, either as individual (mixed) or as fusion formats, was evaluated. Four weeks after the final immunization, mice were challenged intravaginally with 1,500 IFU of C. muridarum. Cervicovaginal washes were taken at day 3, day 6, day 13 and day 13 after infection, and bacterial shedding was measured on HeLa 229 cells. Mice immunized with PBS were used as a negative control, and mice infected with 1,500 IFU of live C. muridarum intravaginally were used as a positive control. The mean Chlamydia IFU±SD is indicated (FIGS. 7A-C).

Different mouse strains showed equal levels of protective effect against Chlamydia muridarum genital tract infection after immunization with PmpE, F, G, H plus MOMP either as individual or as fusion formats.

All citations are hereby incorporated by reference.

The present invention has been described with regard to one or more embodiments. However, it will be apparent to persons skilled in the art that a number of variations and modifications can be made without departing from the scope of the invention as defined in the claims.

1. An immunogenic composition comprising a fusion protein which comprises at least two Chlamydia proteins selected from: Polymorphic membrane protein G (PmpG), Polymorphic membrane protein F (PmpF), Polymorphic membrane protein E (PmpE), Polymorphic membrane protein H (PmpH), Ribosomal protein L6 (RplF), Anti-anti-sigma factor (Aasf), Translocated actin-recruiting phosphoprotein (Tarp), hypothetical protein corresponding to locus tag CT143/TC0420, metalloprotease, insulinase family (CT806/TC0190), hypothetical protein corresponding to locus tag CT538/TC0825, hypothetical protein corresponding to locus tag CT017/TC0285, hypothetical protein corresponding to locus tag CT619, or MOMP, or an immunogenic fragment thereof, together with a physiologically acceptable carrier.

2. The composition of claim 1 wherein the fusion protein comprises PmpG and MOMP.

3. The composition of claim 1 wherein the fusion protein comprises PmpG and PmpF.

4. The composition of claim 1 wherein the fusion protein comprises PmpG and PmpH.

5. The composition of claim 1 wherein the fusion protein comprises PmpE and PmpF.

6. The composition of claim 1 further comprising an adjuvant.

7. The composition of claim 6 wherein the adjuvant is selected from DDA/TDB, DDA/MMG or DDA/MPL.

8. A method for eliciting an immune response against a Chlamydia spp., or component thereof, in an animal comprising administering to the animal an effective amount of the composition of claim 1, thereby eliciting an immune response in the animal.

9. The method of claim 8 wherein the immune response is a cellular immune response.

10. A method for treating or preventing infection by a Chlamydia spp. in an animal comprising administering to the animal an effective amount of the composition of claim 1, thereby treating or preventing infection by the Chlamydia spp. in the animal.

11. The method of claim 8 wherein the Chlamydia spp. is a Chlamydia trachomatis or a Chlamydia muridarum.

12. The method of claim 8 wherein the animal is a human.

13. The method of claim 10 wherein the Chlamydia spp. is a Chlamydia trachomatis or a Chlamydia muridarum.

14. The method of claim 10 wherein the animal is a human.

15-17. (canceled)
